(19) United States
(12) Patent Application Publication
(10) Pub. No.: US 2025/0011767 A1
(43) Pub. Date: Jan. 9, 2025

(54) COMPOSITIONS AND METHODS FOR PRODUCTION OF CIRCULAR NUCLEIC ACID MOLECULES

(71) Applicant: Circularis Biotechnologies, Inc., Oakland, CA (US)

(72) Inventors: Paul A. Feldstein, Oakland, CA (US); Matthew Falkowski, Oakland, CA (US); Christen Yuen, Oakland, CA (US)

(21) Appl. No.: 18/710,373

(22) PCT Filed: Nov. 18, 2022
(86) PCT No.: PCT/US22/50463

§ 371 Date: May 15, 2024

Related U.S. Application Data
(60) Provisional application No. 63/280,944, filed on Nov. 18, 2021.

(51) Int. Cl. C12N 15/11

(52) U.S. Cl. CPC C12N 15/11; C12N 2310/121; C12N 2310/122; C12N 2830/52; C12N 2830/60; C12N 2840/203

(57) ABSTRACT

In certain aspects, the present disclosure provides nucleic acid molecules containing ribozyme catalytic cores for production of circularized RNA containing a nucleic acid sequence of interest, as well as methods of preparation and methods of use.

### RELATED APPLICATIONS

This application claims priority to U.S. Provisional Application 63/208,944, filed Nov. 18, 2021, which is incorporated herein by reference in its entirety.

### BACKGROUND

Circular RNAs lack the free ends necessary for exonuclease-mediated degradation, rendering them resistant to several mechanisms of RNA turnover and granting them extended lifespans as compared to their linear mRNA counterparts (Chen, L. & Yang, L., “Regulation of circRNA biogenesis,” RNA Biology, 12(4):381-388 (2015); Enuka, Y. et al., “Circular RNAs are long-lived and display only minimal early alterations in response to a growth factor,” Nucleic Acids Research, 44(3):1370-1383 (2015)). Circularization therefore can facilitate stabilization of, for example, mRNAs that generally suffer from short half-lives and may therefore improve the overall efficacy of exogenous mRNA in a variety of applications (Kaczmarek, J. C. et al., “Advances in the delivery of RNA therapeutics: from concept to clinical reality,” Genome Medicine, 9(1) (2017); Fink, M. et al., “Improved translation efficiency of injected mRNA during early embryonic development,” Developmental Dynamics, 235(12):3370-3378 (2006); Ferizi, M., et al., “Stability analysis of chemically modified mRNA using micropattern-based single-cell arrays,” Lab Chip, 15(17):3561-3571 (2015)). However, the efficient circularization of in vitro transcribed (IVT) RNA and the purification of circular RNA remain challenging. There remains a need for improved compositions and methods for production of circular RNA molecules.

### SUMMARY

In certain aspects, provided are nucleic acid molecules useful in the generation of circular RNAs containing a sequence of interest, as well as methods of using, generating and purifying such nucleic acid molecules. Accordingly, in certain embodiments, the present disclosure is directed to nucleic acid molecules comprising multiple (e.g., two or more) ribozyme catalytic cores that facilitate the efficient production of circularized RNA.

In some aspects, provided herein are nucleic acid molecules comprising, in 5′ to 3′ order: (i) an upstream ribozyme catalytic core, (ii) an upstream cleavage site, (iii) a central ribozyme catalytic core (e.g., a central hairpin ribozyme catalytic core or a central Varkud satellite (VS) ribozyme catalytic core), (iv) a downstream cleavage site, and (v) a downstream ribozyme catalytic core. In some embodiments, the nucleic acid molecule further comprises a sequence of interest between the upstream cleavage site and the downstream cleavage site. In some embodiments sequences in addition to the central ribosome catalytic core and the sequence of interest (e.g., a second sequence of interest) are also between the upstream cleavage site and the downstream cleavage site. In certain embodiments, a sequence of interest can be any sequence that is to be included in the circular nucleic acid molecule (e.g., it does not need to be a sequence of particular interest). In some embodiments, the upstream ribozyme catalytic core is configured to cleave (and/or is capable of cleaving) the upstream cleavage site to produce an upstream cleaved terminus and the downstream ribozyme catalytic core is configured to cleave (and/or is capable of cleaving) the downstream cleavage site to produce a downstream cleaved terminus. In some embodiments, the central ribozyme catalytic core is configured to join (and/or is capable of joining) the upstream cleaved terminus and the downstream cleaved terminus to produce a circular nucleic acid molecule (e.g., comprising the sequence of interest).

In some aspects, provided herein are nucleic acid molecules comprising, in 5′ to 3′ order: (i) an upstream ribozyme catalytic core, (ii) an upstream cleavage site, (iii) a central ribozyme catalytic core (e.g., a central hairpin ribozyme catalytic core or a central VS ribozyme catalytic core), and (iv) a downstream cleavage site. In some embodiments, the nucleic acid molecule further comprises a sequence of interest between the upstream cleavage site and the downstream cleavage site. In some embodiments sequences in addition to the central ribosome catalytic core and the sequence of interest (e.g., a second sequence of interest) are also between the upstream cleavage site and the downstream cleavage site. In certain embodiments, a sequence of interest can be any sequence that is to be included in the circular nucleic acid molecule (e.g., it does not need to be a sequence of particular interest). In some embodiments, the upstream ribozyme catalytic core is configured to cleave (and/or is capable of cleaving) the upstream cleavage site to produce an upstream cleaved terminus and the central ribozyme catalytic core is configured to cleave (and/or is capable of cleaving) the downstream cleavage site to produce a downstream cleaved terminus. In some embodiments, the central ribozyme catalytic core is configured to join (and/or is capable of joining) the upstream cleaved terminus and the downstream cleaved terminus to produce a circular nucleic acid molecule (e.g., comprising the sequence of interest).

In certain aspects, provided herein are nucleic acid molecules comprising, in 5′ to 3′ order: (i) an upstream cleavage site, (ii) a central ribozyme catalytic core (e.g., a central hairpin ribozyme catalytic core or a central VS ribozyme catalytic core), (iii) a downstream cleavage site, and (iv) a downstream ribozyme catalytic core. In some embodiments, the nucleic acid molecule further comprises a sequence of interest between (i) and (iii). In some embodiments sequences in addition to the central ribosome catalytic core and the sequence of interest (e.g., a second sequence of interest) are also between the upstream cleavage site and the downstream cleavage site. In certain embodiments, a sequence of interest can be any sequence that is to be included in the circular nucleic acid molecule (e.g., it does not need to be a sequence of particular interest). In some embodiments, the central ribozyme catalytic core is configured to cleave (and/or is capable of cleaving) the upstream cleavage site to produce an upstream cleaved terminus and the downstream ribozyme catalytic core is configured to cleave (and/or is capable of cleaving) the downstream cleavage site to produce a downstream cleaved terminus. In some embodiments, the central ribozyme catalytic core is configured to join (and/or is capable of joining) the upstream cleaved terminus and the downstream cleaved terminus to produce a circular nucleic acid molecule (e.g., comprising the sequence of interest).

In some aspects, provided herein are methods of generating circular nucleic acid molecules comprising: (A) generating a nucleic acid molecule comprising, in 5′ to 3′ order: (i) an upstream ribozyme catalytic core, (ii) an upstream cleavage site, (iii) a central ribozyme catalytic core (e.g., a central hairpin ribozyme catalytic core or a central Varkud satellite (VS) ribozyme catalytic core), (iv) a downstream cleavage site, and (v) a downstream ribozyme catalytic core, and, optionally, such nucleic acid molecule also comprising a sequence of interest between the upstream cleavage site and the downstream cleavage site; (B) cleaving the upstream cleavage site with the upstream catalytic core to produce an upstream cleaved terminus; (C) cleaving the downstream cleavage site with the downstream ribozyme catalytic core to produce a downstream cleaved terminus; (D) joining the upstream cleaved terminus and the downstream cleaved terminus with the central ribozyme catalytic core to produce a circular nucleic acid molecule (e.g., comprising the sequence of interest).

In some aspects, provided herein are methods of generating circular nucleic acid molecules comprising: (A) generating a nucleic acid molecule comprising, in 5′ to 3′ order, a nucleic acid molecule comprising, in 5′ to 3′ order: (i) an upstream ribozyme catalytic core, (ii) an upstream cleavage site, (iii) a central ribozyme catalytic core (e.g., a central hairpin ribozyme catalytic core or a central VS ribozyme catalytic core), and (iv) a downstream cleavage site, and, optionally, such nucleic acid molecule comprising a sequence of interest between the upstream cleavage site and the downstream cleavage site; (B) cleaving the upstream cleavage site with the upstream ribozyme catalytic core to produce an upstream cleaved terminus; (C) cleaving the downstream cleavage site with the central ribozyme catalytic core to produce a downstream cleaved terminus; (D) joining the upstream cleaved terminus and the downstream cleaved terminus with the central ribozyme catalytic core to produce a circular nucleic acid molecule (e.g., comprising the sequence of interest).

In some aspects, provided herein are methods of generating circular nucleic acid molecules comprising: (A) generating a nucleic acid molecule comprising, in 5′ to 3′ order, (i) an upstream cleavage site, (ii) a central ribozyme catalytic core (e.g., a central hairpin ribozyme catalytic core or a central VS ribozyme catalytic core), (iii) a downstream cleavage site, and (iv) a downstream ribozyme catalytic core, and, optionally, such nucleic acid molecule comprising a sequence of interest between (i) and (iii); (B) cleaving the upstream cleavage site with the central ribozyme catalytic core to produce an upstream cleaved terminus; (C) cleaving the downstream cleavage site with the downstream ribozyme catalytic core to produce a downstream cleaved terminus; (D) joining the upstream cleaved terminus and the downstream cleaved terminus with the central ribozyme catalytic core to produce a circular nucleic acid molecule (e.g., comprising the sequence of interest).

In some embodiments, the central ribozyme catalytic core is a central hairpin ribozyme catalytic core. In some embodiments, the central ribozyme catalytic core is a ribozyme catalytic core that catalyzes reversible cleavage. In some embodiments, the central ribozyme catalytic core is a central Varkud satellite (VS) ribozyme catalytic core. The central ribozyme catalytic core may be any catalytic core capable of circularization.

In some embodiments, the upstream and/or downstream ribozyme catalytic core is a self-cleaving ribozyme catalytic core. In some embodiments, the upstream and/or downstream ribozyme catalytic core is a hammerhead ribozyme catalytic core. In some embodiments, the upstream and/or downstream ribozyme catalytic core is a hairpin ribozyme catalytic core. In some embodiments, the downstream ribozyme catalytic core is a HDV ribozyme catalytic core. In some embodiments, the upstream and/or downstream ribozyme catalytic core is a VS catalytic core, a twister catalytic core, a twister sister catalytic core, a hatchet catalytic core or a pistol catalytic core.

In some embodiments, the nucleic acid molecules provided herein comprise an upstream catalytic core and a downstream catalytic core. In some embodiments, the upstream ribozyme catalytic core is a hammerhead ribozyme catalytic core and the downstream ribozyme catalytic core is a hammerhead ribozyme catalytic core. In some embodiments, the upstream ribozyme catalytic core is a hairpin ribozyme catalytic core and the downstream ribozyme catalytic core is a hairpin ribozyme catalytic core. In some embodiments, the upstream ribozyme catalytic core is a hammerhead ribozyme catalytic core and the downstream ribozyme catalytic core is a hairpin ribozyme catalytic core. In some embodiments, the upstream ribozyme catalytic core is a hammerhead ribozyme catalytic core and the downstream ribozyme catalytic core is a HDV ribozyme catalytic core. In some embodiments, the upstream ribozyme catalytic core is a hairpin ribozyme catalytic core and the downstream ribozyme catalytic core is a hammerhead ribozyme catalytic core. The upstream ribozyme catalytic core may be a hairpin ribozyme catalytic core and the downstream ribozyme catalytic core may be a HDV ribozyme catalytic core.

In some embodiments, the sequence of interest is located between the upstream cleavage site and the central ribozyme catalytic core. In other embodiments, the sequence of interest is located between the central ribozyme catalytic core and the downstream cleavage site. In some embodiments, the nucleic acid molecules provided herein comprise more than one sequence of interest (e.g., 2, 3, 4, 5, 6, or more sequences of interest). In certain embodiments, one or more of the sequences of interest are located between the upstream cleavage site and the central ribozyme catalytic core. In certain embodiments, one or more of the sequences of interest are located between the central ribozyme catalytic core and the downstream cleavage site. In some embodiments, one or more of the sequences of interest are located between the upstream cleavage site and the central ribozyme catalytic core and one or more of the sequences of interest are located between the upstream cleavage site and the hairpin ribozyme catalytic core.

In some embodiments, the sequence of interest comprises one or more protein coding sequences. In some embodiments, the sequence of interest comprises one or more open reading frames. In certain embodiments, the sequence of interest may comprise an internal ribozyme entry site (IRES), an interfering RNA molecule (e.g., an siRNA or an shRNA), an miRNA binding site, an miRNA, a gRNA (e.g., a sgRNA), an antagomir, an aptamer, a sequence encoding a protein or a polypeptide (e.g., a therapeutic protein, such as a sequence encoding an antibody, a reporter protein), a sequence that binds a RNA binding protein (i.e., a RBP), a spacer sequence, a translation regulation motif, or combinations thereof. In some embodiments, the sequence of interest is at least 250 nucleotides in length, at least 500 nucleotides in length, at least 1000 nucleotides in length, at least 1500 nucleotides in length, at least 2000 nucleotides in length, or at least 2500 nucleotides in length.

In some embodiments, the nucleic acid molecules provided herein comprise a first hairpin insulator sequence and a second hairpin insulator sequence. In some embodiments, each hairpin insulator sequence is 10 base pairs in length. In some embodiments, each hairpin insulator sequence is at least 5, 6, 7, 8, 9, 10, 11, 12, 13, 14, 15, or 20 base pairs in length. In some embodiments, the first hairpin insulator sequence and the second hairpin insulator sequence are the same length. In some embodiments, the first hairpin insulator sequence and the second hairpin insulator sequence are complementary. In some embodiments, the first hairpin insulator sequence is upstream of the sequence of interest. In some embodiments, the second hairpin insulator sequence is downstream of the sequence of interest.

In some embodiments, the nucleic acid molecule comprises an 11 base pair stem between the sequence of interest and the downstream cleavage site.

In some embodiments, the sequence of interest is located between the central ribozyme catalytic core and the downstream cleavage site, and the first hairpin insulator sequence is located between the central ribozyme catalytic core and the sequence of interest, and the second hairpin insulator sequence is located between the sequence of interest and the downstream cleavage site. In certain embodiments, the sequence of interest is located between the upstream cleavage site and the central ribozyme catalytic core, and the first hairpin insulator sequence is located between the upstream cleavage site and the sequence of interest, and the second hairpin insulator sequence is located between the sequence of interest and the central ribozyme catalytic core.

In some embodiments, the nucleic acid molecule further comprises a binding sequence. For example, the binding sequence may be a sequence that is bound by a primer for reverse transcription, a sequence that is bound by a RNA polymerase, a sequence that is bound by a transcription factor, a sequence that is bound by a RNA binding protein, and/or combinations thereof. In some embodiments, the binding sequence is located between the upstream cleavage site and the central ribozyme catalytic core. In some embodiments, the binding sequence is located upstream of the sequence of interest. In some embodiments, the nucleic acid molecule further comprises a promoter sequence. In one embodiment, the nucleic acid molecule comprises an RNA polymerase promoter. The RNA polymerase promoter may be, for example, a T7 virus RNA polymerase promoter, a T6 virus RNA polymerase promoter, a SP6 virus RNA polymerase promoter, a T3 virus RNA polymerase promoter, or a T4 virus RNA polymerase promoter.

In some embodiments, the nucleic acid molecule comprises RNA. In some embodiments, the nucleic acid molecule is an RNA molecule. In some embodiments, the nucleic acid molecule comprises DNA. In some embodiments, the nucleic acid molecule comprises modified nucleotides (e.g., a non-naturally occurring nucleotide, such as those listed in Table 1).

In some of the embodiments disclosed herein, a hammerhead ribozyme catalytic core may be a Schistosoma mansoni hammerhead (HH), a peach latent mosaic viroid HH, a Homo sapiens HH9, or variants thereof. In some embodiments disclosed herein, a hairpin ribozyme catalytic core may be a satellite arabis mosaic virus RNA, a satellite tobacco ringspot virus RNA, a satellite chicory yellow mottle virus RNA, or a variants thereof. In the embodiments disclosed herein, a HDV ribozyme catalytic core may be from the HDV genome, HDV antigenome, or variants thereof.

Also provided herein are circular nucleic acid molecules (e.g., circular RNA molecules) produced by the nucleic acid molecules disclosed herein. Also provided herein are host cells comprising a nucleic acid molecule disclosed herein. In some aspects, provided herein are constructs comprising the nucleic acid molecules disclosed herein.

In some aspects, provided herein are methods of generating circular nucleic acid molecules (e.g., circular RNA molecules) comprising expressing the nucleic acid molecule disclosed herein in a cell (e.g., a mammalian cell, such as a human cell). In some embodiments, provided herein are methods of generating circular nucleic acid molecules (e.g., circular RNA molecules), the method comprising: i) expressing a nucleic acid molecule disclosed herein in a cell, and ii) isolating the circular nucleic acid molecule.

In one aspect, the disclosure provides constructs comprising (i) a central hairpin ribozyme catalytic core, (ii) at least one upstream cleavage site recognized by the central hairpin ribozyme catalytic core, (iii) at least one downstream cleavage site recognized by the central hairpin ribozyme catalytic core, (iv) optionally at least a first ribozyme catalytic core located upstream of the at least one cleavage site of (ii) such that a central hairpin ribozyme catalytic core would functionally interact with cleaved termini, (v) optionally at least a second ribozyme catalytic core located downstream of the central hairpin ribozyme catalytic core and the at least one cleavage site of (iii) such that a central hairpin ribozyme catalytic core would functionally interact with cleaved termini, at least one nucleotide sequence of interest located between (ii) and (i) or between (i) and (iii), and optionally a binding sequence or a promoter sequence. In some embodiments, wherein at least one first ribozyme catalytic core and/or at least one second ribozyme catalytic core is present.

In some embodiments, at least one nucleic acid sequence of interest is located between at least one upstream cleavage site recognized by the central hairpin ribozyme catalytic core and the central hairpin ribozyme catalytic core.

In some embodiments, at least one nucleic acid sequence of interest is located between the central hairpin ribozyme catalytic core and at least one downstream cleavage site recognized by the central hairpin ribozyme catalytic core.

In some embodiments, at least one nucleic acid sequence of interest is located between at least one upstream cleavage site recognized by the central hairpin ribozyme catalytic core and the central hairpin ribozyme catalytic core and at least one nucleic acid of interest is located between the central hairpin ribozyme catalytic core and at least one downstream cleavage site recognized by the central hairpin ribozyme catalytic core.

In some embodiments, the central hairpin ribozyme catalytic core is a self-cleaving ribozyme catalytic core. In some embodiments, the upstream ribozyme catalytic core is a self-cleaving ribozyme catalytic core. In some embodiments, the downstream ribozyme catalytic core is a self-cleaving ribozyme catalytic core.

In some embodiments, the nucleic acid of interest is an internal ribosome entry site (IRES), an interfering RNA molecule, an antagomir, an miRNA binding site, an miRNA, a gRNA (e.g., a sgRNA), a functional RNA, an aptamer, a sequence encoding a reporter gene, a sequence encoding a therapeutic protein (such as a sequence encoding an antibody), a sequence that binds a RNA binding protein (i.e., a RBP), a spacer sequence, a translation regulation motif, and/or combinations thereof.

In some embodiments, the central hairpin ribozyme catalytic core is from satellite arabis mosaic virus RNA, satellite tobacco ringspot virus RNA, satellite chicory yellow mottle virus RNA, or variants thereof.

In some embodiments, at least one first ribozyme catalytic core is present. In some aspects, at least one second ribozyme catalytic core is present. In some aspects, at least one first ribozyme catalytic core and at least one second ribozyme catalytic core is present.

In some embodiments, when at least one first ribozyme catalytic core is present, the first ribozyme catalytic core is a hammerhead ribozyme catalytic core. In other cases, the first ribozyme catalytic core is a hairpin ribozyme catalytic core.

In some embodiments, when at least one second ribozyme catalytic core is present, the second ribozyme catalytic core is a hammerhead, hairpin, or HDV catalytic core.

In some embodiments, when at least one first ribozyme catalytic core as well as at least one second ribozyme catalytic core is present, the first ribozyme catalytic core is a hammerhead catalytic core or a hairpin catalytic core and the second ribozyme catalytic core is a hammerhead catalytic core, a hairpin catalytic core, or a HDV catalytic core.

In some embodiments, when both a first ribozyme catalytic core and a second ribozyme catalytic core is present, the first ribozyme catalytic core is a hammerhead catalytic core and the second ribozyme catalytic core is a hairpin catalytic core. In another aspect, the first ribozyme catalytic core is a hammerhead catalytic core and the second ribozyme catalytic core is also a hammerhead catalytic core. In yet another aspect, the first ribozyme catalytic core is a hairpin catalytic core and the second ribozyme catalytic core is a HDV catalytic core. In still another aspect, the first ribozyme catalytic core is a hairpin catalytic core and the second ribozyme catalytic core is a hairpin catalytic core. In yet another aspect, the first ribozyme catalytic core is a hairpin catalytic core and the second ribozyme catalytic core is a hammerhead catalytic core. In a further aspect, the first ribozyme catalytic core is a hammerhead catalytic core and the second ribozyme catalytic core is a HDV catalytic core.

In some embodiments, the hairpin catalytic core is from satellite arabis mosaic virus RNA, satellite tobacco ringspot virus RNA, satellite chicory yellow mottle virus RNA, or variants thereof.

In some embodiments, the hammerhead catalytic core is a hammerhead ribozyme catalytic core from any hammerhead ribozyme and variants thereof, such as the catalytic core from Schistosoma mansoni hammerhead ribozyme, peach latent mosaic viroid hammerhead ribozyme, Homo sapiens hammerhead ribozyme 9 (1119), or variants thereof.

In some embodiments, the HDV ribozyme catalytic core is from the HDV genome, HDV antigenome, or variants thereof. In some aspects, provided herein are circular RNAs resulting from the construct disclosed herein. Also provided herein, are host cells comprising the construct disclosed herein.

In some embodiments, the upstream ribozyme cleavage site and the downstream ribozyme cleavage site contain only the native P and D region sequences associated with the native P′ and D′ region sequences of the central hairpin ribozyme catalytic core. In other aspects, the upstream ribozyme cleavage site contains the native D region sequences that will associate with the native D′ region sequence associated with the central hairpin ribozyme catalytic core or the downstream ribozyme cleavage site contains the native P region sequences that will associate with the P′ region sequence associated with the central hairpin ribozyme catalytic core. In still other aspects, the P region sequences upstream of the central hairpin catalytic core are the native sequences associated with the first ribozyme and/or the D region sequences downstream of the central hairpin catalytic core are the native sequence associated with the second ribozyme. In yet other cases, the upstream D and/or D′ region sequences associated with the central hairpin ribozyme catalytic core are altered from the native D and D′ region sequences of the central hairpin ribozyme catalytic core. In still other cases the downstream D and D′ region sequences associated with the central hairpin ribozyme catalytic core are altered from the native D and D′ region sequences of the downstream central hairpin ribozyme catalytic core.

In some embodiments, the length of the stem sequence adjacent to the P sequence downstream of the central catalytic core contains the native length of stem sequence associated with the central catalytic core in satTRSV. In other cases the stem region sequence adjacent to the PD regions of the central catalytic core contain an altered length of stem region sequence as compared to the native length of stem region sequence associated with the central catalytic core.

In some embodiments, also provided herein is circular RNA generated from the constructs of the present disclosure. In some embodiments, the also provided is a method of generating circular RNA from the constructs of the present disclosure.

### BRIEF DESCRIPTION OF THE DRAWINGS

FIG. 1 illustrates the folding associated with full length or truncated negative strand of satellite tobacco ringspot virus RNA ((-)sTRSV RNA) when circularized. FIG. 1A shows the folding of the full-length negative strand of the satellite tobacco ringspot virus RNA. P (UGACA) represents the proximal region of the ribozyme cleavage site, which binds to the complement P′ (GUCA). D represents the distal region of the ribozyme cleavage site (GUCCUGUUU), D′ represents the complementary sequence that anneals to D (GACAAA), Arrow shows the position of the cleavage site, and the gray area delineated by a dashed line indicates the minimal hairpin ribozyme catalytic core. FIG. 1B represents the folding of the hairpin ribozyme region associated with a minimal 2-way junction truncated negative strand of satellite tobacco ringspot virus RNA with the arrow indicating the cleavage site. Region B represents the part of the catalytic region of the central ribozyme formed by Helix 3 (H3), Helix 4 (H4) and the sequence between them. Region A represents the part of the catalytic region of the central ribozyme formed by Helix 1 (H1), Helix 2 (H2), and the sequence in between. The right hand portion of H2 and the nucleotide immediately after it represents the P region of the ribozyme cleavage site and the left hand portion of H2 represents the P′ region of the hairpin ribozyme core. The three nucleotides immediately after the arrow and the right hand portion of H1 represents the D region of the ribozyme cleavage site and the left hand portion of H1 represents the D′ region. FIG. 1C represents the folding of the hairpin ribozyme region associated with a 4-way junction truncated negative strand of satellite tobacco ringspot virus RNA with the arrow indicating the cleavage site. H1-H4 are the same as in FIG. 1B. H5 and H6 are helices that connect H3 and H2 and stabilize the overall structure and improve its cleavage and ligation. The length of the sequence comprising the catalytic core can vary, but retains a geometry which allows interaction of the A loop and the B loop. The Circ2.0 construct (see FIG. 6) also contains an 11 bp stem sequence which acts to stabilize the structure as a 4 way junction.

FIG. 2 is a schematic of the process of self-circularization of a mini-monomer. P represents the proximal region of the ribozyme cleavage site, D represents the distal region of the ribozyme cleavage site, and MinHp represents the minimal hairpin ribozyme catalytic core. While hairpin ribozymes cleave at the junction of the P and D regions, specifically within the P region one base into the A loop closest to the P/P′ stem (see FIG. 1A), the cleavage by these ribozymes tends to be highly reversible. Similarly, the circularization reaction which can occur after complete cleavage is also reversible.

FIG. 3 is a schematic of one mini-monomer construct (Circ1a) containing a HDV ribozyme located downstream from the MinHp catalytic core. P represents the proximal region of the ribozyme cleavage site, D represents the distal region of the ribozyme cleavage site, Insulator hairpin part A (GGCGCGCCCC; SEQ ID NO:60) refers to a sequence that is substantially complementary to Insulator hairpin part B (GGGGTGTGCC (SEQ ID NO:61) in a DNA construct or GGGGUGUGCC (SEQ ID NO:62) in an RNA construct), the arching arrows indicate the cleavage site acted upon by the MinHp and the HDV ribozymes.

FIG. 4 is a schematic of one mini-monomer construct containing a second ribozyme positioned upstream of MinHp. D represents the distal region of the ribozyme cleavage site, P represents the proximal region of the ribozyme cleavage site, the Insulator and stem portions are as described in FIG. 3, and the arching arrows indicate the cleavage site acted upon by the second and the MinHp ribozymes. FIG. 4 illustrates the general organization of an exemplary construct having a second ribozyme sequence located upstream of the central catalytic core sequence. In this situation the second ribozyme cleaves the construct and removes itself from the construct, leaving the D region still attached to the truncated construct. The central catalytic core cleaves at the PD junction located downstream of it, releasing the D region. After the cleavages, the resulting remaining construct has both a D region and a P region and can then undergo circularization. The second ribozyme of FIG. 4 can be any type of ribozyme.

FIG. 5 shows the structure of a mini-monomer construct (Circ-upMinHpvar) containing a satellite arabis mosaic virus RNA minimal hairpin catalytic core (sArMV MinHp) located upstream of a satellite tobacco ringspot virus RNA minimal hairpin catalytic core (satTRSV MinHp) as well as in vitro transcription results for the Circ-upMinHpvar and a Circ2.0 construct. As the D region of the ribozyme cleavage site between the upstream sArMV MinHp and central satTRSV MinHp are recognized by both, the D′ regions can be of different lengths. In this case, the sArMV MinHp has a longer D′ to increase the stability of its interaction with the ribozyme cleavage site and increase the cleavage of the site.

FIG. 6 shows two mini-monomer constructs: a Circ2.0 and a Circ-upMinHpvar. To the top right, the figure shows Circ2.0, which is a version of the construct in FIG. 2, using a satellite tobacco ringspot virus minimal catalytic core (satTRSV MinHp). To the bottom right, the figure shows the structure of Circ-upMinHpvar which contains a satellite arabis mosaic virus minimal catalytic core (sArMV MinHp) located upstream of a satellite tobacco ringspot virus minimal catalytic core (satTRSV MinHp). To the left, the figure shows in vitro transcription results. As the D region of the ribozyme cleavage site between the upstream sArMV MinHp and central satTRSV MinHp in Circ-upMinHpvar are recognized by both, the D′ regions can be of different lengths (as shown in FIG. 6, D′=8 for sArMV and D′=7 for satTRSV). In this case, the sArMV MinHp has a longer D′ to increase the stability of its interaction with the upstream ribozyme cleavage site and increase the cleavage of the site. FIG. 5 and FIG. 6 illustrate the structure of a construct where the second ribozyme is the catalytic core of a hairpin ribozyme. In this instance, the catalytic core of sArMV along with its native stem loops.

FIG. 7 shows the structure of a mini-monomer construct (Circ-upHsHH) containing a Homo sapiens hammerhead ribozyme located upstream of a satellite tobacco ringspot virus minimal catalytic core (satTRSV MinHp) and a construct (Circ-upSmHH) containing a Schistosoma mansoni hammerhead ribozyme located upstream of satellite tobacco ringspot virus minimal catalytic core (satTRSV MinHp), as well as in vitro transcription results for those constructs and a Circ2.0 construct. D′ 7/7, 7/4, 4/7, etc. refers to the number of base pairs between the different D and D′ sequences of the D region in the first ribozyme cleavage site and the second ribozyme cleavage site. For example, D′ 7/4 has 7 bases pairs between the upstream D and the central hairpin ribozyme D′ and 4 base pairs between the downstream D and the central hairpin ribozyme D′. The 5+4 represents a D/D′ interaction with two regions of 5 and 4 base pairs with unbase paired region in between. FIG. 7 illustrates the structure of a construct where the second ribozyme is a hammerhead ribozyme. In this instance either the Homo sapiens or the Schistosoma mansoni hammerhead ribozymes were used, although any known hammerhead ribozyme could be substituted. When upstream hammerhead ribozymes are used, the hammerhead removes itself from the construct, leaving the D region attached to the truncated construct. The central catalytic core then cleaves the PD region downstream of it, releasing the D region. After the cleavages, the resulting remaining construct has both a D region and a P region and can then undergo circularization.

FIG. 8 shows the structure of a mini-monomer construct having a Homo sapiens hammerhead ribozyme (HsHH) located upstream of a satellite tobacco ringspot virus minimal catalytic core (MinHp) and a third ribozyme located downstream of the MinHP. D represents the distal region of the MinHP ribozyme cleavage site, P represents the proximal region of the MinHp ribozyme cleavage site, the Insulator and stem portions are as described in FIG. 3, and the arching arrows indicate the cleavage site acted upon by the HsHH and third ribozymes.

FIG. 9 shows the structure of a mini-monomer construct (Circ-dnPLMVHH) having a peach latent mosaic viroid hammerhead ribozyme (PLMV-HH) located downstream of a satellite tobacco ringspot virus minimal catalytic core (satTRSV MinHp) and a construct (Circ-upHsHH/dnPLMVHH) containing a Homo sapiens hammerhead ribozyme (HsHH) located upstream of a satellite tobacco ringspot virus minimal catalytic core (satTRSV MinHp) and a peach latent mosaic viroid hammerhead ribozyme (PLMV-HH) located downstream of the satTRSV MinHp, as well as in vitro transcription results. P represents the proximal region of the MinHP cleavage site, D represents the distal region of the MinHp cleavage site.

FIG. 10 shows the structure of a mini-monomer construct (Circ-upHsHH/dnMinHPvar) having a Homo sapiens hammerhead ribozyme (HH) located upstream of a satellite tobacco ringspot virus minimal catalytic core (satTRSV MinHp) and a satellite arabis mosaic virus minimal catalytic core (sArMV MinHp) located downstream of the satTRSV MinHp as well as a construct having a Homo sapiens hammerhead ribozyme (HsHH) located upstream of a satellite tobacco ringspot virus minimal catalytic core (satTRSV MinHp) and a Schistosoma mansoni hammerhead ribozyme located downstream of the satTRSV MinHp. Diff D′ indicates a D′ in the sArMV MinHp with the sequence (GAGACTC), which is different from the D′ of the central satTRSV MinHp. This should allow the diff D′ sequence to interact with the downstream ribozyme cleavage site without interfering with or being interfered with by the upstream D or D′ sequences. D represents the distal region of the MinHP ribozyme cleavage site, and P represents the proximal region of the MinHp ribozyme cleavage site. In vitro transcription results are also shown for a construct having the HsHH ribozyme without (i.e., a control) and with the ribozyme located downstream of the satTRSV MinHp.

FIG. 11 shows the results of in vitro transcription of the constructs shown in FIG. 10.

FIG. 12 shows the structure of a Circ-upHsHH construct having a Homo sapiens hammerhead ribozyme (HsHH) located upstream of a satellite tobacco ringspot virus minimal catalytic core (satTRSV MinHp) with stem lengths of 5, 7, 9 and 11 bp and the sequences used (SEQ ID NO:1-4). Nucleotides in bold font anneal to form the stem. C/L ratio refers to the amount of circularized (“C”) product compared to linear (“L”) product generated. “2 C” and “2 L” refer to double mini-monomer products. In vitro transcription results are also presented.

FIG. 13 illustrates one construct used for inserting a nucleotide of interest in between the Insulator stems. The elements shown, reading left to right, are a Homo sapiens hammerhead ribozyme (HsHH), a D region, a minimal hairpin catalytic core (MinHp), an insulator stem, a region in which the nucleotide of interest is inserted, an insulator stem, an 11 bp stem, a P region, and a D region.

FIG. 14 is a gel showing the various forms of RNA generated from IVT of construct Circ3.2 and Circ3.1-HDV mini-monomers, each of which contains an insert of interest. Lanes are labeled using the clone number preceded by “S,” “M,” or “L” which represent a range of small (S; 200-500 nucleotides), medium (M; 800-1200 nucleotides), or large (L; 1500-2000 nucleotides) insert sizes. “C” refers to circular mini-monomer, “2 C” refers to a dimer circular mini-monomer, “L” refers to a linear mini-monomer, “2 L” refers to a linear dimer mini-monomer, “1°” refers to a primary transcript, “Ø1” refers to a transcript containing HsHH but lacking D, and “Ø2” refers to a transcript lacking HsHH but containing D (+10 nucleotides). Size values that lack “˜” have been confirmed by sequencing. Due to compression within the gel, large sized transcripts were not easily identifiable, consequently some lanes do not have a complete identification of transcripts produced.

FIG. 15 is a gel showing the various forms of RNA generated from IVT of construct Circ3.1-MinHP-sArMV mini-monomer, which contains an insert of interest. Lanes are labeled using the clone number preceded by “S,” “M,” or “L” which represent a range of small (S), medium (M), or large (L) insert sizes. “C” refers to circular mini-monomer, “2 C” refers to a dimer circular mini-monomer, “L” refers to a linear mini-monomer, “2 L” refers to a linear dimer mini-monomer, “1°” refers to a primary transcript, “Ø1” refers to a transcript containing HsHH (+57 nucleotides) but lacking MinHp-sArMV, and “Ø2” refers to a transcript lacking HsHH but containing MinHp-sArMV (+81 nucleotides). Size values that lack “˜” have been confirmed by sequencing. Due to compression within the gel, large sized transcripts were not easily identifiable, consequently some lanes do not have a complete identification of transcripts produced.

FIG. 16 is a gel showing the various forms of RNA generated from IVT of construct Circ3.1-SmHH mini-monomer, which contains an insert of interest. Lanes are labeled using the clone number preceded by “S,” “M,” or “L” which represent a range of small (S), medium (M), or large (L) insert sizes. “C” refers to circular mini-monomer, “2 C” refers to a dimer circular mini-monomer, “L” refers to a linear mini-monomer, “2 L” refers to a linear dimer mini-monomer, “1°” refers to a primary transcript, “Ø1” refers to a transcript containing HsHH (+57 nucleotides) but lacking SmHH, and “Ø2” refers to a transcript lacking HsHH but containing SmHH (+75 nucleotides). Size values that lack “˜” have been confirmed by sequencing. Due to compression within the gel, large sized transcripts were not easily identifiable, consequently some lanes do not have a complete identification of transcripts produced.

FIG. 17 shows an exemplary nucleic acid molecule according to the disclosure herein.

FIG. 18 shows a 4% (29:1), 7M urea gel of in vitro transcriptions of medium sized circRNAs containing IRES and luciferase coding sequences (CDS). Gel showing the various forms of RNA generated from IVT of construct Circ3.4 derivatives including different IRES' and Gaussia luciferase coding sequences. L and C represent the linear and circular monomeric form of the fully processed RNA. 2 L and 2 C represent the linear and circular dimeric form of the fully processed RNA. In some lanes, the 2 C bands are not labeled due to either the small amount present or difficulty in precisely locating it.

FIG. 19 shows a 4% (19:1), 7M urea gel of the in vitro transcriptions of the medium sized circRNAs containing IRES and luciferase CDS seen in FIG. 18.

FIG. 20 shows luciferase activity in HEK293T (kidney), HepG2 (liver), and HCT116 (colon) cells transfected with exemplary nucleic acid molecules as illustrated in FIG. 17 containing IRES sequences from Table 3. Those IRES' shown in Table 3 and FIGS. 18 and 19, but not shown in FIG. 20 were excluded due to low or zero apparent luciferase expression in HEK293T cells. The CVB3 IRES is from coxsackie virus B3 and was used as the positive control.

FIG. 21 shows a subset of FIG. 7 that compares a construction with upstream and downstream cleavage sites for the central hairpin ribozyme (Circ2.0) and a construct where the upstream cleavage site has been changed to require the activity of an upstream Homo sapiens hammerhead (HsHH) (Circ3.2) to improve upstream cleavage. The addition of the upstream hammerhead improved processing and circularization as evidenced by the amount of circular RNA present in the Circ3.2 lane compared to the Circ2.0 lane.

### DETAILED DESCRIPTION

In certain aspects, provided are nucleic acid molecules useful in the generation of circular RNAs containing a sequence of interest, as well as methods of using, generating and purifying such nucleic acid molecules. Accordingly, in certain embodiments, the present disclosure is directed to nucleic acid molecules comprising multiple (e.g., two or more) ribozyme catalytic cores that facilitate the efficient production of circularized RNA.

Therefore, in certain aspects provided herein are nucleic acid molecules comprising, in 5′ to 3′ order: (i) an upstream ribozyme catalytic core, (ii) an upstream cleavage site, (iii) a central ribozyme catalytic core, (iv) a downstream cleavage site, and (v) a downstream ribozyme catalytic core. In some embodiments, the nucleic acid molecule further comprises a sequence of interest between (ii) and (iv). In some embodiments, the upstream ribozyme catalytic core is configured to cleave (and/or is capable of cleaving) the upstream cleavage site to produce an upstream cleaved terminus. In some embodiments, the downstream ribozyme catalytic core is configured to cleave (and/or is capable of cleaving) the downstream cleavage site to produce a downstream cleaved terminus. In some embodiments, the central ribozyme catalytic core is configured to join (and/or is capable of joining) the upstream cleaved terminus and the downstream cleaved terminus to produce a circular nucleic acid molecule comprising the sequence of interest.

In some aspects, provided herein are nucleic acid molecules comprising, in 5′ to 3′ order: (i) an upstream ribozyme catalytic core, (ii) an upstream cleavage site, (iii) a central ribozyme catalytic core, and (iv) a downstream cleavage site. In some embodiments, the nucleic acid molecule further comprises a sequence of interest between the upstream cleavage site and the downstream cleavage site (e.g., between (ii) and (iv)). In some embodiments, the upstream ribozyme catalytic core is configured to cleave (and/or is capable of cleaving) the upstream cleavage site to produce an upstream cleaved terminus and the central ribozyme catalytic core is configured to cleave (and/or is capable of cleaving) the downstream cleavage site to produce a downstream cleaved terminus. In some embodiments, the central ribozyme catalytic core is configured to join (and/or is capable of joining) the upstream cleaved terminus and the downstream cleaved terminus to produce a circular nucleic acid molecule comprising the sequence of interest.

In other aspects, provided herein are nucleic acid molecules comprising, in 5′ to 3′ order: (i) an upstream cleavage site, (ii) a central ribozyme catalytic core, (iii) a downstream cleavage site, and (iv) a downstream ribozyme catalytic core. In some embodiments, the nucleic acid molecule further comprises a sequence of interest between (i) and (iii). In some embodiments, the central ribozyme catalytic core is configured to cleave (and/or is capable of cleaving) the upstream cleavage site to produce an upstream cleaved terminus and the downstream ribozyme catalytic core is configured to cleave (and/or is capable of cleaving) the downstream cleavage site to produce a downstream cleaved terminus. In some embodiments, the central ribozyme catalytic core is configured to join (and/or is capable of joining) the upstream cleaved terminus and the downstream cleaved terminus to produce a circular nucleic acid molecule comprising the sequence of interest.

In certain embodiments, the nucleic acid molecule described herein may be a construct. Constructs can be DNA and/or RNA. In some embodiments, the construct can comprise the following operably linked polynucleotide elements:
a central hairpin ribozyme catalytic core;
at least one upstream cleavage site recognized by the central hairpin ribozyme catalytic core;
at least one downstream cleavage site recognized by the central hairpin ribozyme catalytic core;
optionally at least a first ribozyme catalytic core located upstream of the at least one cleavage site of (ii) such that a central hairpin ribozyme catalytic core would functionally interact with cleaved termini;
optionally at least a second ribozyme catalytic core located downstream of the central hairpin ribozyme catalytic core and the at least one cleavage site of (iii) such that a central hairpin ribozyme catalytic core would functionally interact with cleaved termini;
at least one nucleotide of interest located between ii and i or between i and iii; and
optionally a binding sequence or a promoter sequence,
wherein at least one first ribozyme catalytic core and/or at least one second ribozyme catalytic core is present.

In some embodiments, the polynucleotide elements are operably linked in the 5′ to 3′ direction. For example, in one of the following manners:
(a) optionally a promoter or binding sequence located upstream of the first ribozyme catalytic core;
optionally at least a first ribozyme catalytic core located upstream of at least one upstream cleavage site recognized by the upstream ribozyme catalytic core such that a central hairpin ribozyme catalytic core would functionally interact with cleaved termini;
at least one upstream cleavage site recognized by the central hairpin catalytic core;
optionally a binding sequence or a promoter;
optionally a nucleic acid sequence of interest;
optionally a nucleic acid sequence of interest and a binding sequence or a promoter;
a central hairpin ribozyme catalytic core;
optionally a binding sequence or promoter;
optionally a nucleic acid sequence of interest;
optionally a nucleic acid sequence of interest and a binding sequence or a promoter;
at least one downstream cleavage site recognized by the central hairpin ribozyme catalytic core; and
optionally at least a second ribozyme catalytic core located downstream of the at least one cleavage site recognized by the central hairpin ribozyme catalytic core such that a central hairpin ribozyme catalytic core would functionally interact with cleaved termini.

### Definitions

For convenience, certain terms employed in the specification, examples, and appended claims are collected here.

The term “amino acid” is intended to embrace molecules, whether natural or synthetic, which include both an amino functionality and an acid functionality and capable of being included in a polymer of naturally occurring amino acids. Example amino acids include naturally occurring amino acids; analogs, derivatives and congeners thereof; amino acid analogs having variant side chains; and stereoisomers of any of any of the foregoing.

The term “nucleic acid molecule” refers to a polymeric form of nucleotides, either deoxyribonucleotides or ribonucleotides, or analogs thereof. The terms include of single-stranded or double-stranded molecules comprised of nucleic acid bases. As such, the term includes, and may be used interchangeably with “plasmids”, “constructs”, or “vectors.” Nucleic acid molecules may have any three-dimensional structure.

The terms “polynucleotide” and “nucleic acid” are used interchangeably. They refer to a polymeric form of nucleotides, either deoxyribonucleotides or ribonucleotides, or analogs thereof. The terms include single-stranded or double-stranded molecules comprised of nucleic acid bases. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

The phrase “central catalytic core” as used herein refer to a catalytic ribozyme sequence in a central ribozyme. In some embodiments, the central ribozyme catalytic core is a central hairpin ribozyme catalytic core. In some embodiments, the central ribozyme catalytic core is a central VS ribozyme catalytic core. The central ribozyme catalytic core may include the P/P′ sequences and the D/D′ sequence which flank the catalytic core A loop, and a catalytic core B loop flanked by Helix3 (H3) and Helix4 (H4). The size of the central ribozyme catalytic core can vary from about 40 nucleotides to any desired size. Examples of useful sizes include, without limitation, at least 10, at least 20, at least 30, at least 40, at least 45, at least 50, at least 60, at least 70, at least 80, at least 90, at least 100, at least 120, at least 140, at least 160, at least 180, at least 200, at least 225, at least 250, at least 275, at least 300, at least 350, at least 400, at least 450, at least 500, at least 600, at least 700, at least 800, at least 900, at least 1000 or more nucleotides. The central ribozyme catalytic core may comprise 40 to 1000 nucleotides. The phrase “catalytic core A loop” as used herein refers to the loop of sequence occurring between the stem generated by the annealing of the P and P′ regions and the stem generated by the annealing of the D and D′ regions.

The phrase “catalytic core B loop” as used herein refers to the loop of sequence occurring between the stem generated by the annealing of the sequence proximal to the P′ sequence, also known as H3 and the stem generated at the opposite end of the loop, also known as H4.

The phrase “central ribozyme” refers to the ribozyme and its catalytic core (e.g., “central catalytic core,” and “central ribozyme catalytic core”) capable of and/or configured for circularization of the nucleic acid molecule in which it is located. In some embodiments, the circularization occurs through RNA-mediated unimolecular ligation. The central ribozyme may contain a nucleic acid insert or sequence of interest. In some embodiments, the central ribozyme is located between at least one additional ribozyme or ribozyme catalytic core 5′ to the central ribozyme catalytic core (e.g., an “upstream ribozyme”) and/or at least one additional ribozyme or ribozyme catalytic core 3′ to the central ribozyme (e.g., a “downstream ribozyme”).

The term “D” or “D site” refers to the cleavage sequence/region that is 3′ or distal to the upstream or downstream ribozyme cleavage site.

The term “P” or “P site” refers to the sequence/region that is 5′ or proximal to the downstream or upstream ribozyme cleavage site.

The term “downstream” refers to sequence that is 3′ to a particular sequence or ribozyme. For example, the phrase “downstream ribozyme” refers to a separate ribozyme located 3′ to the central ribozyme.

The term “upstream” refers to sequence that is 5′ to a particular sequence or ribozyme. For example, the phrase “upstream ribozyme” refers to a separate ribozyme that is located 5′ to the central ribozyme.

The phrase “dual ribozyme” refers to a ribozyme capable of both cleavage and ligation reactions.

The term “hairpin ribozyme” refers to an RNA motif that catalyzes self-RNA processing reactions that modify/rearranges its own structure. In some embodiments, the ribozyme folds into a secondary structure that includes two domains, each consisting of two short base paired helices separated by an internal loop. The two domains are covalently joined via a phosphodiester linkage such that in the active state they lie parallel to one another. Both cleavage and end joining reactions are mediated by the ribozyme motif and lead to a mixture of interconvertible linear and circular satellite RNA molecules. These reactions process the large multimeric RNA molecules generated by rolling circle replication. Examples of hairpin ribozymes are found in the satellite RNA of, without limitation, tobacco ringspot virus (sTRSV or satTRSV), chicory yellow mottle virus (sCYMV), and arabis mosaic virus (sArMV).

The term “HDV” refers to the genome and anti-genome ribozymes associated with the Hepatitis Delta Virus which requires its ribozyme activities to replicate in its host.

The term “hammerhead ribozyme” refers to an RNA motif that catalyzes reversible cleavage and litigation reactions at a specific site within an RNA molecule. Generally, the minimal sequence required for self-cleavage of the hammerhead ribozyme includes about 13 conserved or invariant core nucleotides that are flanked by three helices/stems (stems I, II, and III) that are separated by short linkers of conserved sequences. Exemplary hammerhead ribozymes can be found in the database set forth in Stenz and Sullivan (2012) Investigative Ophthalmology & Visual Science 53: 5126. Examples of hammerhead ribozymes include those from, without limitation, avocado sunblotch viroid, Schistosoma satellite DNA, Dolichopoda, Arabidopsis thaliana, Homo sapiens (HsHH), Schistosoma mansoni (SmHH) and a peach latent mosaic viroid (denoted herein as “PLMV-HH”).

Sequences are “substantially identical” or “variants thereof” if they have a specified percentage of nucleic acid residues or amino acid residues that are the same (i.e., at least 60% identity, e.g., at least 65%, 70%, 75%, 80%, 85%, 90%, 95%, 96%, 97%, 98%, or 99% identity to a reference sequence (e.g., SEQ ID NOs: 1-62) over a specified region (or the whole reference sequence when not specified)), when compared and aligned for maximum correspondence over a comparison window, or designated region as measured using any sequence comparison algorithm known in the art (GAP, BESTFIT, BLAST, Align, FASTA, and TFASTA in the Wisconsin Genetics Software Package, Genetics Computer Group (GCG), 575 Science Dr., Madison, Wis.), Karlin and Altschul Proc. Natl. Acad. Sci. (U.S.A.) 87:2264-2268 (1990) set to default settings, or by manual alignment and visual inspection (see, e.g., Ausubel et al., Current Protocols in Molecular Biology (1995-2014). Optionally, the identity exists over a region that is at least about 5, 10, 15, 20, 25, 30, 35, 40, 45, 50, 75, 100, 200, 300, 400, 500, 600, 800, 1000, or more, nucleic acids in length, or any value there between, or over the full-length of the sequence.

The phrase “mini-monomer cassette” refers to a polynucleotide sequence comprising a central ribozyme catalytic core and upstream and downstream ribozyme cleavage sites, such that when transcribed into RNA, the ribozyme catalytic core self-cleaves the mini-monomer cassette at the upstream and downstream ribozyme cleavage sites out of the context of a longer polynucleotide. The 5′ and 3′ ends of the excised polynucleotide ligate to form a circularized polynucleotide. Optionally, a “mini-monomer cassette” may contain an upstream ribozyme and/or a downstream ribozyme designed to cleave the transcribed RNA such that the product of said cleavages would have appropriate sequences and terminal structures to be circularized by the central ribozyme catalytic core. The term “P/P′ stem” refers to the stem generated from the annealing of the P and P′ sequences.

The term “PMLV” as used herein refers to the peach latent mosaic viroid.

The term “ribozyme” refers to an RNA molecule having catalytic activity that cleaves or modifies themselves, targeted RNAs, or targeted DNAs.

The phrase “ribozyme catalytic core” refers to a sequence within the ribozyme capable of carrying out cleavage, modification, and/or ligation of an RNA or DNA molecule.

The phrase “ribozyme cleavage site” refers to a sequence site recognized and cleaved by a ribozyme catalytic core. The phrase “ribozyme ligation site” refers to fragments of a ribozyme cleavage site with appropriate terminal structures that can be ligated by a central ribozyme catalytic core.

The term “Varkud satellite (VS) ribozyme” includes any ribozyme embedded in VS RNA. VS RNA exists as satellite RNA found in mitochondria of Varkud-1C and other strains of Neurospora. It includes ribozymes comprising five helical sections, organized by two three-way junctions.

### Nucleic Acid Molecules

In certain aspects, provided herein are nucleic acid molecules useful for efficient circularization of RNA, with or without a sequence of interest.

In some embodiments, the nucleic acid molecules described herein are synthetic and/or recombinant. Synthetic and/or recombinant nucleic acid molecules can be made by any known method in the art. Synthetic nucleic acid molecules can be generated as either RNA or DNA, and generated using standard techniques, such as “DNA printing” (see, for example Palluk (2018) Nature Biotechnology 36: 645-650) or with dedicated devices from companies such as Kilobaser (Graz, Austria) or CureVac (Boston, MA). Synthetic nucleic acid molecules can also be ordered from companies such as Twist Biosciences (South San Francisco, CA), DNA Script (South San Francisco, CA), and Integrated DNA Technologies (Coralville, IA). While the sequences listed in the Sequence Listing are primarily listed as DNA, after converting thymine to uracil these same sequences can be used for RNA constructs.

Recombinant nucleic acid molecules, such as recombinant constructs, are generated using standard molecular biology techniques, such as those set forth in Green and Sambrook (Molecular Cloning: A Laboratory Manual, Fourth Edition, ISBN-13: 978-1936113415).

The nucleic acid molecules can be comprised wholly of naturally occurring nucleic acids, or in certain aspects can contain one or more nucleic acid analogues or derivatives. The nucleic acid analogues can include backbone analogues and/or nucleic acid base analogues and/or utilize non-naturally occurring base pairs. Illustrative artificial nucleic acids that can be used in the present constructs include, without limitation, nucleic backbone analogs peptide nucleic acids (PNA), morpholino and locked nucleic acids (LNA), bridged nucleic acids (BNA), glycol nucleic acids (GNA) and threose nucleic acids (TNA). Nucleic acid base analogues that can be used in the present constructs include, without limitation, fluorescent analogs (e.g., 2-aminopurine (2-AP), 3-Methylindole (3-MI), 6-methyl isoxanthoptherin (6-MI), 6-MAP, pyrrolo-dC and derivatives thereof, furan-modified bases, 1,3-Diaza-2-oxophenothiazine (tC), 1,3-diaza-2-oxophenoxazine); non-canonical bases (e.g., inosine, thiouridine, pseudouridine, dihydrouridine, queuosine and wyosine), 2-aminoadenine, thymine analogue 2,4-difluorotoluene (F), adenine analogue 4-methylbenzimidazole (Z), isoguanine, isocytosine; diaminopyrimidine, xanthine, isoquinoline, pyrrolo[2,3-b]pyridine; 2-amino-6-(2-thienyl)purine, pyrrole-2-carbaldehyde, and universal bases (e.g., 2′ deoxyinosine (hypoxanthine deoxynucleotide) derivatives, nitroazole analogues). Non-naturally occurring base pairs that can be used in the present nucleic acid molecules include, without limitation, isoguanine and isocytosine; diaminopyrimidine and xanthine; 2-aminoadenine and thymine; isoquinoline and pyrrolo[2,3-b]pyridine; 2-amino-6-(2-thienyl)purine and pyrrole-2-carbaldehyde; two 2,6-bis(ethylthiomethyl)pyridine (SPy) with a silver ion; pyridine-2,6-dicarboxamide (Dipam) and a mondentate pyridine (Py) with a copper ion.

The nucleic acid molecules disclosed herein may have a modified base. In some embodiments, a “modified base” is a ribonucleotide base of uracil, cytosine, adenine, or guanine that possesses a chemical modification from its normal structure. For example, one type of modified base is a methylated base, such as N6-methyladenosine (m6A). A modified base may also be a substituted base, meaning the base possesses a structural modification that renders it a chemical entity other than uracil, cytosine, adenine, or guanine. For example, pseudouridine is one type of substituted RNA base. Table 1 below provides a list of exemplary modified bases that may be present in a nucleic acid molecule described herein.

TABLE 1
List of Exemplary Base Modifications

| Abbreviation | Chemical name |
|---|---|
| m¹acp³Y | 1-methyl-3-(3-amino-3-carboxypropyl) pseudouridine |
| m¹A | 1-methyladenosine |
| m¹G | 1-methylguanosine |
| m¹I | 1-methylinosine |
| m¹Y | 1-methylpseudouridine |
| m¹Am | 1,2′-O-dimethyladenosine |
| m¹Gm | 1,2′-O-dimethylguanosine |
| m¹Im | 1,2′-O-dimethylinosine |
| m²A | 2-methyladenosine |
| ms²io⁶A | 2-methylthio-N⁶-(cis-hydroxyisopentenyl) adenosine |
| ms²hn⁶A | 2-methylthio-N⁶-hydroxynorvalyl carbamoyladenosine |
| ms²i⁶A | 2-methylthio-N⁶-isopentenyladenosine |
| ms²m⁶A | 2-methylthio-N⁶-methyladenosine |
| ms²t⁶A | 2-methylthio-N⁶-threonyl carbamoyladenosine |
| s²Um | 2-thio-2′-O-methyluridine |
| s²C | 2-thiocytidine |
| s²U | 2-thiouridine |
| Am | 2′-O-methyladenosine |
| Cm | 2′-O-methylcytidine |
| Gm | 2′-O-methylguanosine |
| Im | 2′-O-methylinosine |
| Ym | 2′-O-methylpseudouridine |
| Um | 2′-O-methyluridine |
| Ar(P) | 2′-O-ribosyladenosine (phosphate) |
| Gr(p) | 2′-O-ribosylguanosine (phosphate) |
| acp³U | 3-(3-amino-3-carboxypropyl)uridine |
| m³C | 3-methylcytidine |
| m³Y | 3-methylpseudouridine |
| m³U | 3-methyluridine |
| m³Um | 3,2′-O-dimethyluridine |
| imG-14 | 4-demethylwyosine |
| s⁴U | 4-thiouridine |
| chm⁵U | 5-(carboxyhydroxymethyl)uridine |
| mchm⁵U | 5-(carboxyhydroxymethyl)uridine methyl ester |
| inm⁵s²U | 5-(isopentenylaminomethyl)-2-thiouridine |
| inm⁵Um | 5-(isopentenylaminomethyl)-2′-O-methyluridine |
| inm⁵U | 5-(isopentenylaminomethyl)uridine |
| nm⁵s²U | 5-aminomethyl-2-thiouridine |
| ncm⁵Um | 5-carbamoylmethyl-2′-O-methyluridine |
| ncm⁵U | 5-carbamoylmethyluridine |
| cmnm⁵Um | 5-carboxymethylaminomethyl-2′-O-methyluridine |
| cmnm⁵s²U | 5-carboxymethylaminomethyl-2-thiouridine |
| cmnm⁵U | 5-carboxymethylaminomethyluridine |
| cm⁵U | 5-carboxymethyluridine |
| f⁵Cm | 5-formyl-2′-O-methylcytidine |
| f⁵C | 5-formylcytidine |
| hm⁵C | 5-hydroxymethylcytidine |
| ho⁵U | 5-hydroxyuridine |
| mcm⁵s²U | 5-methoxycarbonylmethyl-2-thiouridine |
| mcm⁵Um | 5-methoxycarbonylmethyl-2′-O-methyluridine |
| mcm⁵U | 5-methoxycarbonylmethyluridine |
| mo⁵U | 5-methoxyuridine |
| m⁵s²U | 5-methyl-2-thiouridine |
| mnm⁵se²U | 5-methylaminomethyl-2-selenouridine |
| mnm⁵s²U | 5-methylaminomethyl-2-thiouridine |
| mnm⁵U | 5-methylaminomethyluridine |
| m⁵C | 5-methylcytidine |
| m⁵D | 5-methyldihydrouridine |
| m⁵U | 5-methyluridine |
| tm⁵s²U | 5-taurinomethyl-2-thiouridine |
| tm⁵U | 5-taurinomethyluridine |
| m⁵Cm | 5,2′-O-dimethylcytidine |
| m⁵Um | 5,2′-O-dimethyluridine |
| preQ₁ | 7-aminomethyl-7-deazaguanosine |
| preQ₀ | 7-cyano-7-deazaguanosine |
| m⁷G | 7-methylguanosine |
| G⁺ | archaeosine |
| D | dihydrouridine |
| oQ | epoxyqueuosine |
| galQ | galactosyl-queuosine |
| OHyW | hydroxywybutosine |
| I | inosine |
| imG2 | isowyosine |
| k²C | lysidine |
| manQ | mannosyl-queuosine |
| mimG | methylwyosine |
| m²G | N²-methylguanosine |
| m²Gm | N²,2′-O-dimethylguanosine |
| m^{2,7}G | N²,7-dimethylguanosine |
| m^{2,7}Gm | N²,7,2′-O-trimethylguanosine |
| m² ₂G | N²,N²-dimethylguanosine |
| m² ₂Gm | N²,N²,2′-O-trimethylguanosine |
| m^{2,2,7}G | N²,N²,7-trimethylguanosine |
| ac⁴Cm | N⁴-acetyl-2′-O-methylcytidine |
| ac⁴C | N⁴-acetylcytidine |
| m⁴C | N⁴-methylcytidine |
| m⁴Cm | N⁴,2′-O-dimethylcytidine |
| m⁴ ₂Cm | N⁴,N⁴,2′-O-trimethylcytidine |
| io⁶A | N⁶-(cis-hydroxyisopentenyl)adenosine |
| ac⁶A | N⁶-acetyladenosine |
| g⁶A | N⁶-glycinylcarbamoyladenosine |
| hn⁶A | N⁶-hydroxynorvalylcarbamoyladenosine |
| i⁶A | N⁶-isopentenyladenosine |
| m⁶t⁶A | N⁶-methyl-N⁶-threonylcarbamoyladenosine |
| m⁶A | N⁶-methyladenosine |
| t⁶A | N⁶-threonylcarbamoyladenosine |
| m⁶Am | N⁶,2′-O-dimethyladenosine |
| m⁶ ₂A | N⁶,N⁶,-dimethyladenosine |
| m⁶ ₂Am | N⁶,N⁶,2′-O-trimethyladenosine |
| o₂yW | peroxywybutosine |
| Y | pseudouridine |
| Q | queuosine |
| OHyW | undermodified hydroxywybutosine |
| cmo⁵U | uridine 5-oxyacetic acid |
| mcmo⁵U | uridine 5-oxyacetic acid methyl ester |
| yW | wybutosine |
| imG | wyosine |

In some embodiments, a ribozyme disclosed herein (e.g., the central ribozyme) is a hairpin ribozyme. In some embodiments, the hairpin ribozyme comprises a central catalytic core (e.g., the central hairpin ribozyme catalytic core). Exemplary hairpin ribozymes include, without limitation, those ribozymes found in the satellite RNA of tobacco ringspot virus (sTRSV or satTRSV), chicory yellow mottle virus (sCYMV), and arabis mosaic virus (sArMV).

In some embodiments, one or more ribozymes are located downstream and/or upstream of the central hairpin ribozyme catalytic core.

In some embodiments, a ribozyme disclosed herein (e.g., the central ribozyme) is a VS ribozyme. In some embodiments, the VS ribozyme comprises a central catalytic core (e.g., the central VS ribozyme catalytic core).

In some embodiments, one or more ribozymes are located downstream and/or upstream of the central VS ribozyme catalytic core.

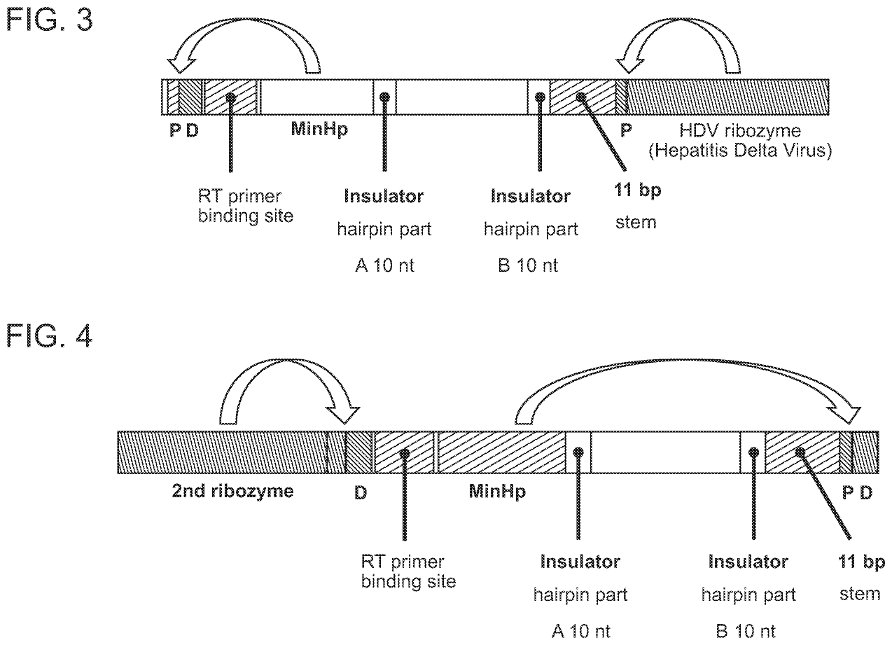

In some embodiments, a HDV ribozyme is placed downstream of a central ribozyme catalytic core. The HDV ribozyme is capable of irreversibly cleaving at a P/HDV junction upstream of the HDV ribozyme and removes itself from the nucleic acid molecule, leaving the P region attached to the truncated nucleic acid molecule. The central catalytic core cleaves at the PD junction located upstream of it, releasing the P region. After the cleavages, the resulting remaining nucleic acid molecule has both a D region and a P region and can then undergo circularization (see FIG. 3).

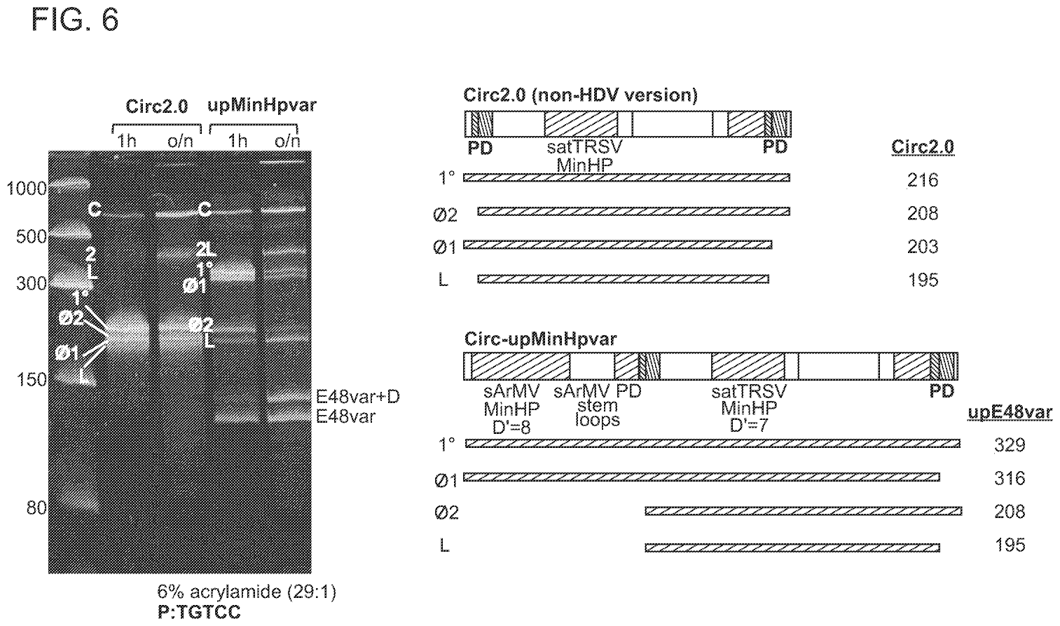

In some embodiments, the nucleic acid molecules disclosed herein include a Circ2.0 construct or a nucleic acid molecule derived from a Circ2.0 construct. The Circ2.0 construct (see FIG. 6) comprises a reverse transcriptase (RT) binding site located between the PD regions and the central catalytic core.

In some embodiments, the nucleic acid molecule comprises a central ribozyme comprising a catalytic core. In some embodiments, the nucleic acid molecule comprises a downstream ribozyme comprising a catalytic core. In some embodiments, the nucleic acid molecule comprises an upstream ribozyme comprising a catalytic core. In some embodiments, the nucleic acid molecule comprises a central ribozyme comprising a catalytic core, an upstream ribozyme comprising a catalytic core, and a downstream ribozyme comprising a catalytic core. In some embodiments, the efficiency of cleavage without compromising the circularization reaction is improved by upstream and/or downstream ribozymes.

In some embodiments, the upstream ribozyme catalytic core is a hammerhead ribozyme catalytic core. In some embodiments, the upstream ribozyme catalytic core is a hairpin ribozyme catalytic core. In some embodiments, the upstream ribozyme catalytic core is a VS catalytic core, a twister catalytic core, a twister sister catalytic core, a hatchet catalytic core or a pistol catalytic core.

In some embodiments, the downstream ribozyme catalytic core is a hammerhead ribozyme catalytic core. In some embodiments, the downstream ribozyme catalytic core is a hairpin ribozyme catalytic core. In some embodiments, the downstream ribozyme catalytic core is a HDV ribozyme catalytic core. In some embodiments, the downstream ribozyme catalytic core is a VS catalytic core, a twister catalytic core, a twister sister catalytic core, a hatchet catalytic core or a pistol catalytic core.

In some embodiments, the upstream ribozyme catalytic core can be the same ribozyme catalytic core as the downstream ribozyme catalytic core. For example, in some embodiments, the upstream ribozyme catalytic core is a hammerhead ribozyme catalytic core and the downstream ribozyme catalytic core is a hammerhead ribozyme catalytic core. In some embodiments, the upstream ribozyme catalytic core is a hairpin ribozyme catalytic core and the downstream ribozyme catalytic core is a hairpin ribozyme catalytic core.

In some embodiments, the upstream ribozyme catalytic core and the downstream ribozyme catalytic core are different ribozyme catalytic cores. For example, in some embodiments, the upstream ribozyme catalytic core is a hammerhead ribozyme catalytic core and the downstream ribozyme catalytic core is a hairpin ribozyme catalytic core. In some embodiments, the upstream ribozyme catalytic core is a hammerhead ribozyme catalytic core and the downstream ribozyme catalytic core is a HDV ribozyme catalytic core.

In some embodiments, the upstream ribozyme catalytic core is a hairpin ribozyme catalytic core and the downstream ribozyme catalytic core is a hammerhead ribozyme catalytic core. The upstream ribozyme catalytic core may be a hairpin ribozyme catalytic core and the downstream ribozyme catalytic core is a HDV ribozyme catalytic core.

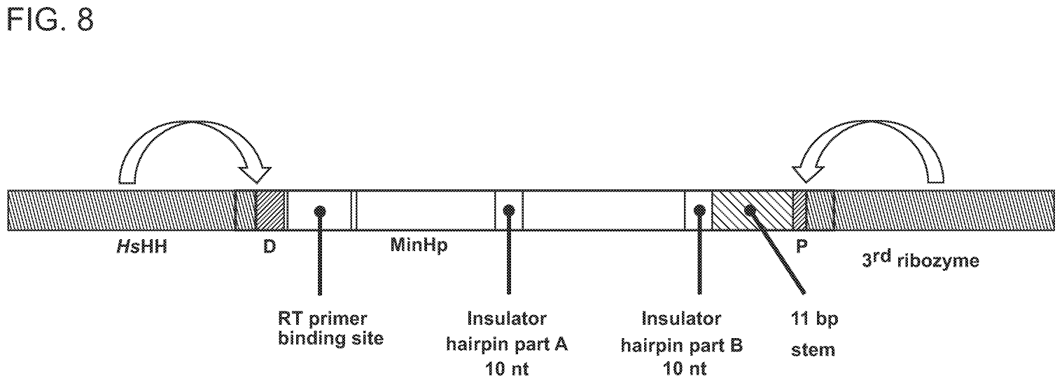

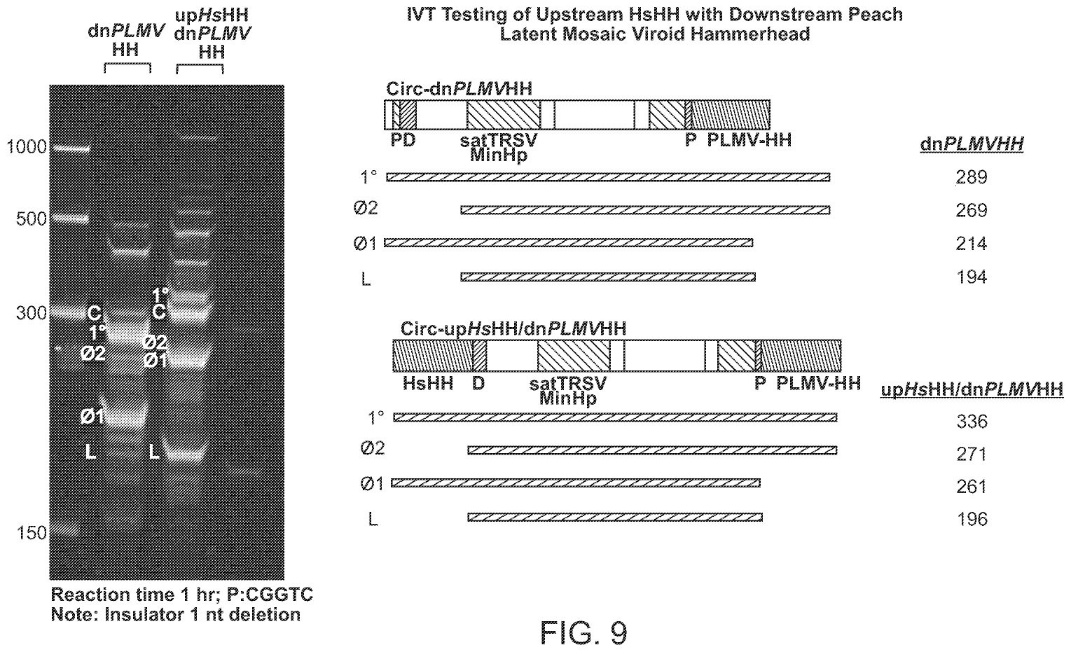

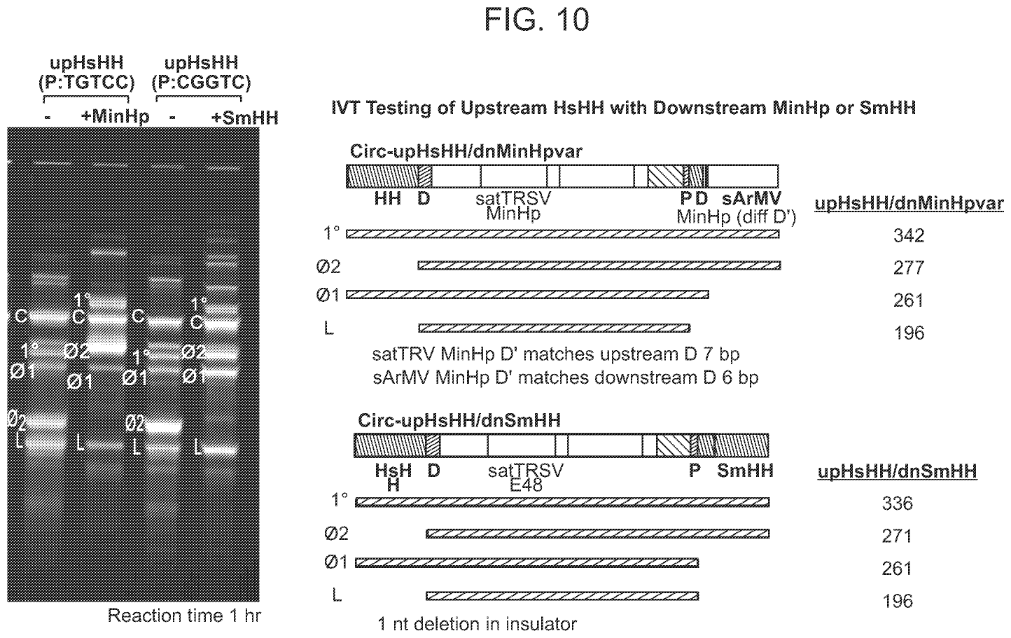

In some embodiments, the nucleic acid molecule comprises multiple ribozymes, such as at least three, at least four, at least five, or at least six ribozymes. FIG. 8 illustrates an exemplary nucleic acid molecule disclosed herein comprising the Homo sapiens hammerhead ribozyme upstream from the central catalytic core and a third ribozyme located downstream of the central catalytic core. The third (or any additional) ribozyme can be any type of ribozyme known in the art. FIG. 9 and FIG. 10 illustrate constructs where the third ribozyme is a hammerhead ribozyme (FIG. 9) or the catalytic core of a hairpin ribozyme (FIG. 10). Hammerhead ribozymes can be any known hammerhead ribozyme. Nucleic acid molecules can also contain more than one ribozyme located upstream and/or downstream of the central catalytic core. The upstream ribozyme removes itself from the nucleic acid molecule, leaving the D region attached to the truncated nucleic acid molecule. The downstream ribozyme removes itself, leaving the P region attached to the truncated nucleic acid molecule. After the cleavages, the resulting remaining nucleic acid molecule has both a D region (i.e., located at the upstream cleaved termini) and a P region (i.e., located at the downstream cleaved termini) and can then undergo circularization.

In some embodiments, a ribozyme disclosed herein is a self-cleaving ribozyme. Self-cleaving ribozymes are known in the art. The cleavage activities of self-cleaving ribozymes can be dependent upon divalent cations, pH, and base-specific mutations, which can cause changes in the nucleotide arrangement and/or electrostatic potential around the cleavage site (see, e.g., Weinberg et al., “New Classes of Self-Cleaving Ribozymes Revealed by Comparative Genomics Analysis,” Nat. Chem. Biol. 11(8): 606-610 (2015) and Lee et al., “Structural and Biochemical Properties of Novel Self-Cleaving Ribozymes,” Molecules 22(4):E678 (2017), which are hereby incorporated by reference in their entirety). Therefore, any nucleic acid molecule disclosed herein would be utilized under experimental or therapeutic conditions known in the art.

Suitable self-cleaving ribozymes include, but are not limited to, hammerhead, hairpin, hepatitis Delta Virus (“HDV”), Neurospora Varkud Satellite (“VS”), twister, twister sister, hatchet, pistol, and engineered synthetic ribozymes, and derivatives thereof (see, e.g., Harris et al., “Biochemical Analysis of Pistol Self-Cleaving Ribozymes,” RNA 21(11):1852-8 (2015), which is hereby incorporated by reference in its entirety). As such included herein are ribozyme catalytic cores that are Neurospora Varkud Satellite (“VS”) catalytic cores, twister catalytic cores, twister sister catalytic cores, hatchet catalytic cores, pistol catalytic cores, and engineered synthetic ribozyme catalytic cores. For example, the upstream and/or the downstream catalytic core can be a Neurospora Varkud Satellite (“VS”) catalytic core, a twister catalytic core, a twister sister catalytic core, a hatchet catalytic core, a pistol catalytic core, or an engineered synthetic ribozyme catalytic core.

Hairpin ribozymes refer to an RNA motif that catalyzes self-RNA processing reactions that modify/rearranges its own structure. In some embodiments, the hairpin ribozyme folds into a secondary structure that includes two domains, each consisting of two short base paired helices separated by an internal loop. The two domains, in the active state, lie parallel to one another. Examples of hairpin ribozymes are found in the satellite RNA of, without limitation, tobacco ringspot virus (sTRSV or satTRSV), chicory yellow mottle virus (sCYMV), and arabis mosaic virus (sArMV).

Hammerhead ribozymes may be composed of structural elements generally including three helices, referred to as stem I, stem II, and stem III, and joined at a central core of single strand nucleotides. Hammerhead ribozymes may also contain loop structures extending from some or all of the helices. These loops are numbered according to the stem from which they extend (e.g., loop I, loop II, and loop III).

Twister ribozymes comprise three essential stems (P1, P2, and P4), with up to three additional ones (P0, P3, and P5) of optional occurrence. Three different types of Twister ribozymes have been identified depending on whether the termini are located within stem P1 (type P1), stem P3 (type P3), or stem P5 (type P5) (see, e.g., Roth et al., “A Widespread Self-Cleaving Ribozyme Class is Revealed by Bioinformatics,” Nature Chem. Biol. 10(1):56-60 (2014)). The fold of the Twister ribozyme is predicted to comprise two pseudoknots (T1 and T2, respectively), formed by two long-range tertiary interactions (see Gebetsberger et al., “Unwinding the Twister Ribozyme: from Structure to Mechanism,” WIREs RNA 8(3):e1402 (2017), which is hereby incorporated by reference in its entirety).

Twister sister ribozymes are similar in sequence and secondary structure to twister ribozymes. In particular, some twister RNAs have P1 through P5 stems in an arrangement similar to twister sister and similarities in the nucleotides in the P4 terminal loop exist. However, these two ribozyme classes cleave at different sites, twister sister ribozymes do not appear to form pseudoknots via Watson-Crick base pairing (which occurs in twister ribozymes).

Pistol ribozymes are characterized by three stems: P1, P2, and P3, as well as a hairpin and internal loops. A six-base-pair pseudoknot helix is formed by two complementary regions located on the P1 loop and the junction connecting P2 and P3; the pseudoknot duplex is spatially situated between stems P1 and P3 (Lee et al., “Structural and Biochemical Properties of Novel Self-Cleaving Ribozymes,” Molecules 22(4):E678 (2017), which is hereby incorporated by reference in its entirety).

In some embodiments, the ribozymes provided herein may include naturally-occurring (wildtype) ribozymes and modified ribozymes, e.g., ribozymes containing one or more modifications, which can be addition, deletion, substitution, and/or alteration of at least one (or more) nucleotide. Such modifications may result in the addition of structural elements (e.g., a loop or stem), lengthening or shortening of an existing stem or loop, changes in the composition or structure of a loop(s) or a stem(s), or any combination of these. As described herein, modification of the nucleotide sequence of naturally occurring self-cleaving ribozymes can increase or decrease the ability of a ribozyme to autocatalytically cleave its RNA. In one embodiment, each of the ribozymes is modified to comprise a non-natural or modified nucleotide. In some embodiments, one or more of the ribozymes disclosed herein are modified.

In some embodiments, the P and D regions of the central ribozyme are optimized for more efficient cleavage by the upstream and downstream ribozymes. Given the simpler requirements of the D/D′ interaction needed for central hairpin ribozyme activity, changes to the sequences in the D and/or D′ region can assist in maintaining the tertiary interactions required for efficient ribozyme (e.g., hammerhead) activity. Alternative P sequences can have better cleavage efficiency and give the RNA formed better resistance to RNAse R.

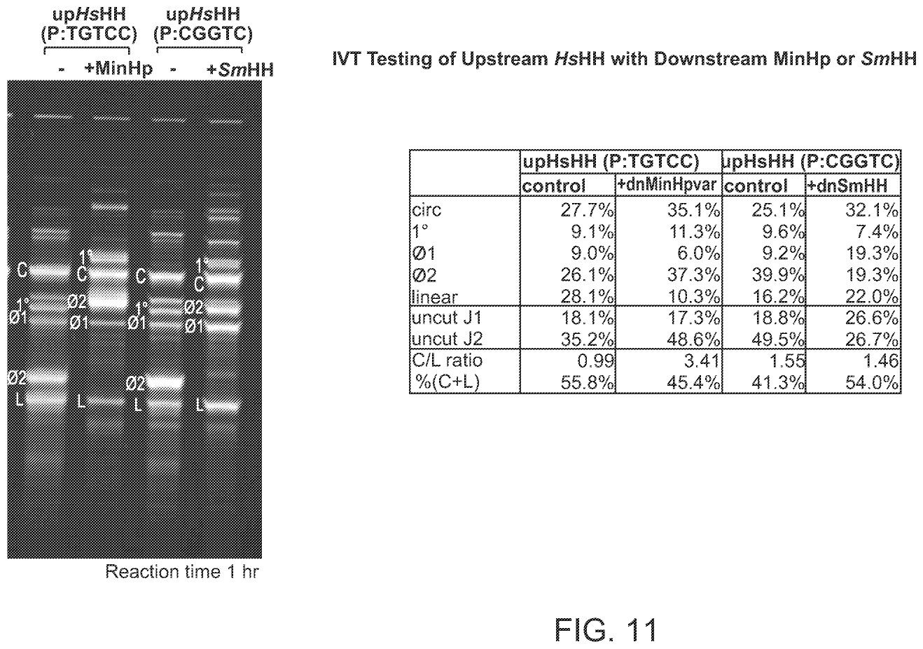

In some embodiments, the P sequence may be 5 nucleotides in length and can be any combination of nucleotides, resulting in a total of 1,024 potential functioning sequences. In some embodiments the P sequence is TGTCC, CAGAC, CGGTA, CGGTC, CAGTA, and CTCTG (see, for example, FIGS. 10 and 11). With respect to the P′ sequence, this sequence may be 4 nucleotides in length because the first base is not essential for pairing with the P sequence. Consequently, there are a total of 256 potential functioning P′ sequences. In some embodiments, the following sequences are used: GACA, TCTG, ACCG, ACTG, and AGAG.

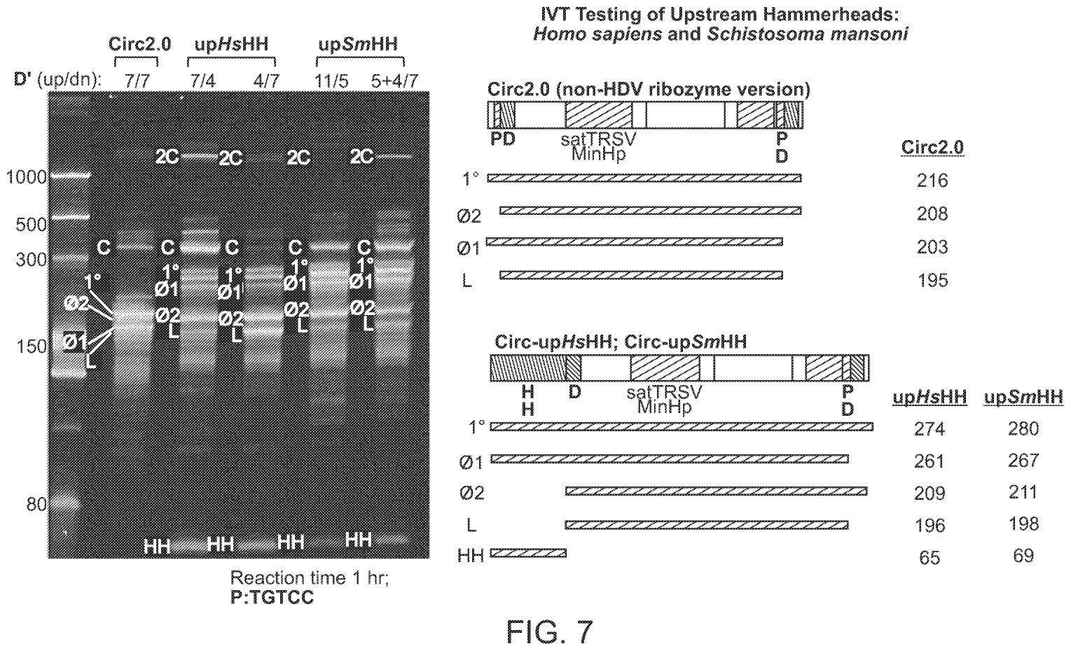

In some embodiments, the D region is GTCGAGTCTC, GTCGAGTCTCC (SEQ ID NO: 5), GTCGAGTATCGG (SEQ ID NO:6), and GTCGAGTCCAATCC (SEQ ID NO: 7). In some embodiments, the D′ region is GAGACTC, TGGACTC, and AGTACTC. This is illustrated in FIGS. 7 and 10.

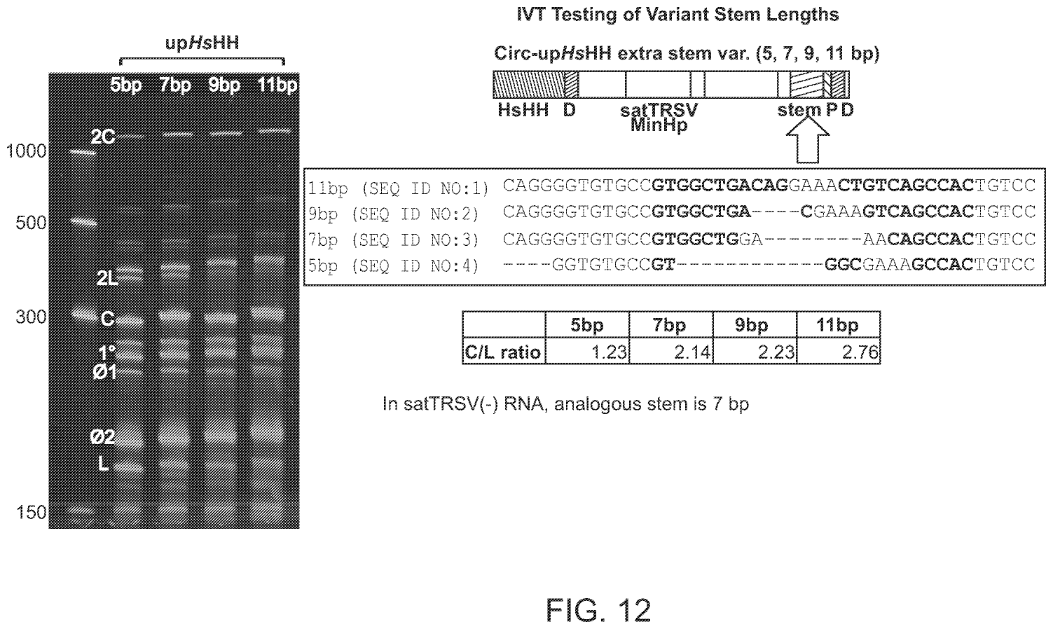

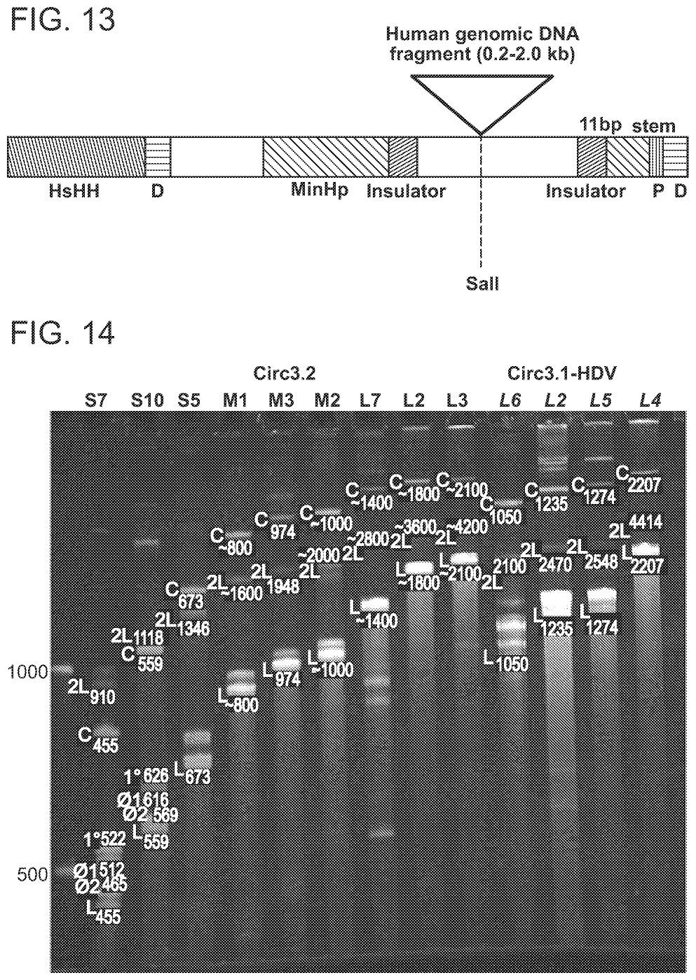

The stem sequence adjacent to the downstream P region can be any sequence that self-anneals to form a stem and can be any length or can be absent. Oftentimes, an 11 bp stem is used (see FIG. 12).

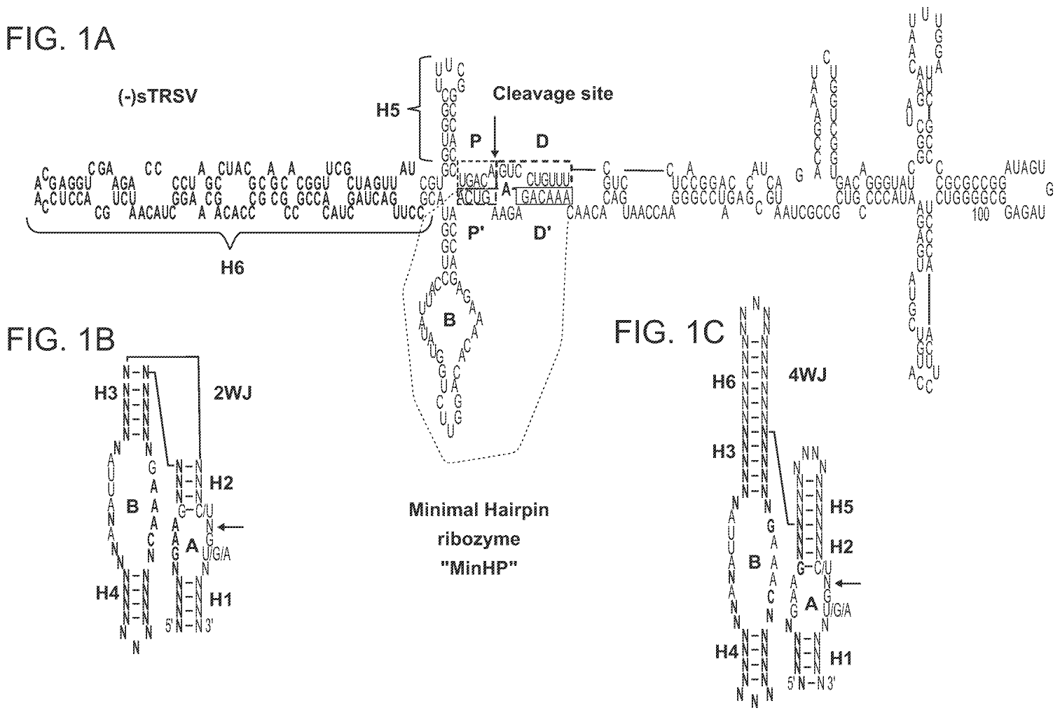

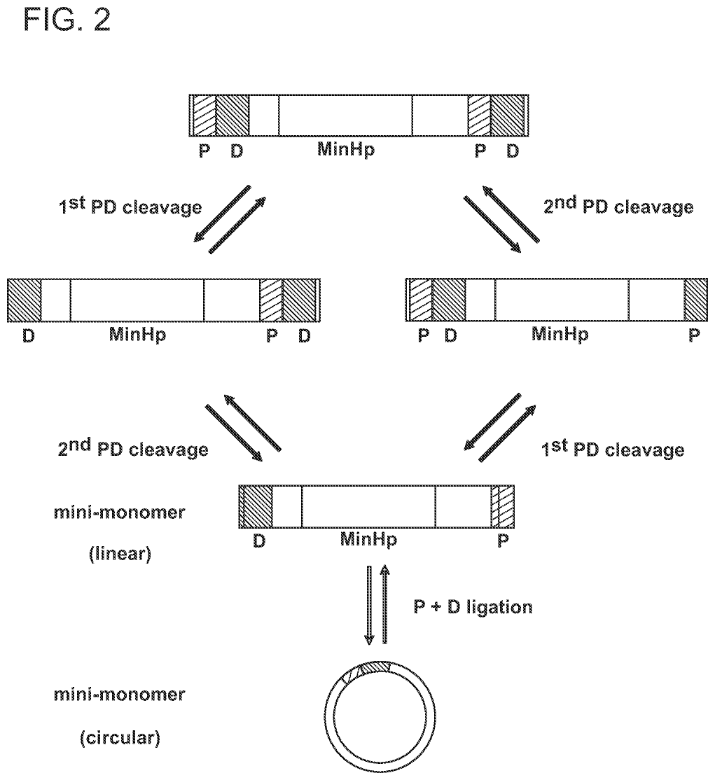

In some embodiments, a ribozyme disclosed herein comprises at least one insulator hairpin sequence (e.g., a first and second insulator hairpin sequence). The insulator sequence may be a 10 nucleotide sequence (an example is shown as “Insulator hairpin part A” in FIG. 3) and located downstream of the central catalytic core. The first insulator hairpin sequence may be complementary to a 10 nucleotide second hairpin insulator sequence which, when annealed, generates a stem and a loop containing any sequence located between the first and second hairpin insulator sequences. In some embodiments, the sequence of interest is located between the first and second hairpin insulator sequences. In some embodiments, the first insulator hairpin sequence and the second insulator hairpin sequence are complementary (see, as an example, FIG. 3, showing 10 nucleotide insulator hairpin part A sequence and the insulator hairpin part B sequence, which is complementary and, when annealed, create the insulator stem). In some embodiments, the first insulator hairpin sequence and the second insulator hairpin sequence are complementary and create a stem when annealed. For example, the first insulator hairpin sequence and the second insulator hairpin sequence may have perfect complementary. In some embodiments, the first insulator hairpin sequence and the second insulator hairpin sequence have partial complementary. Any sequence can be used as long as its complement is present in the other insulator hairpin sequence. In addition, the length of the insulator stem can be shorter or longer as long as it is capable of stabilizing the 4-way intersection that is depicted in FIG. 1C.

In some embodiments, the nucleic acid molecules described herein comprise a first hairpin insulator sequence and a second hairpin insulator sequence. In some embodiments, each hairpin insulator sequence is at least 5 base pairs in length (e.g., at least 6, 7, 8, 9, 10, 11, 12, 13, 14, 15, 20, 25 or 30 base pairs in length).

If the sequence of interest is located between the central ribozyme catalytic core and the downstream cleavage site, the first hairpin insulator sequence may, in some embodiments, be located between the central ribozyme catalytic core and the sequence of interest, and the second hairpin insulator sequence may be located between the sequence of interest and the downstream cleavage site.

If the sequence of interest is located between the upstream cleavage site and the central ribozyme catalytic core, the first hairpin insulator sequence may, in some embodiments, be located between the upstream cleavage site and the sequence of interest, and the second hairpin insulator sequence may be located between the sequence of interest and the central ribozyme catalytic core.

In some embodiments, the central ribozyme comprises one or more ligation sequences (e.g., a P and D sequence). As used herein, the phrase “ligation sequence” refers to a sequence complementary to another sequence, which enables the formation of Watson-Crick base pairing to form suitable substrates for ligation by a ligase, e.g., an RNA ligase. The first ligation sequence and the second ligation sequence may each, independently, comprise at least 1, 2, 3, 4, 5, 6, 7, 8, 9, 10, 11, or 12 additional nucleotides to promote base-pairing with each other. the first ligation sequence and the second ligation sequence are substrates for an RNA ligase. According to one embodiment, the RNA ligase is RtcB. RtcB is not present in all lower organisms, but molecules with similar activities are present. In other words, there are molecules that ligate ends similar to the ligation activity of RtcB. RtcB (or other functionally similar molecules) may be overexpressed to maximize circular nucleic acid expression.

The purpose of the ligation sequence is to assist in circularization of the nucleic acid molecule, to protect the nucleic acid molecule from degradation and, therefore, ultimately enhance expression of the sequence of interest. In some embodiments, the nucleic acid molecule provided herein is configured to circularize (and/or is capable of circularizing) without the ligation sequences.

Methods of producing a ribozyme targeted to a target sequence are known in the art. Ribozymes may be designed as described in PCT Publication No. WO 93/23569 and PCT Publication No. WO 94/02595, each of which is hereby incorporated by reference in its entirety, and synthesized to be tested in vitro and in vivo, as described therein.

In some embodiments, the nucleic acid molecule comprises a sequence of interest. The sequence of interest can fall into any category of biological molecules. Some examples of suitable nucleic acids include, without limitation, an RNA for silencing, an internal ribosome entry site (IRES), an aptamer, a coding sequence, a functional sequence, a barcode sequence, and/or combinations thereof. The sequence of interest may comprise an internal ribozyme entry site (IRES), an interfering RNA molecule (e.g., an siRNA or an shRNA), an miRNA binding site, an miRNA, a gRNA (e.g., a sgRNA), an antagomir, an aptamer, a sequence encoding a protein or a polypeptide (e.g., a therapeutic protein, such as a sequence encoding an antibody, or a reporter protein), a sequence that binds a RNA binding protein (i.e., a RBP), a spacer sequence, a translation regulation motif, or combinations thereof.

In some embodiments, the IRES sequence is an IRES sequence of picornavirus (e.g., a bat, macaca, rabbit, or a guinea fowl picornavirus), enterovirus (e.g., an EV J or an EV96 enterovirus virus), encephalomyelitis virus (e.g., theilers murine encephalomyelitis virus), Taura syndrome virus, Triatoma virus, Theiler's encephalomyelitis virus, simian Virus 40, Solenopsis invicta virus 1, Rhopalosiphum padi virus, Reticuloendotheliosis virus, fuman poliovirus 1, Plautia stali intestine virus, Kashmir bee virus, Human rhinovirus 2, Homalodisca coagulata virus-1, Human Immunodeficiency Virus type 1, Homalodisca coagulata virus-1, Himetobi P virus, Hepatitis C virus, Hepatitis A virus, Hepatitis GB virus, foot and mouth disease virus, Human enterovirus 71, Equine rhinitis virus, Ectropis obliqua picorna-like virus, Encephalomyocarditis virus (EMCV), Drosophila C Virus, Crucifer tobamo virus, Cricket paralysis virus, Bovine viral diarrhea virus 1, Black Queen Cell Virus, Aphid lethal paralysis virus, Avian encephalomyelitis virus, Acute bee paralysis virus, Hibiscus chlorotic ringspot virus, Classical swine fever virus, Human FGF2, Human SFTPA1, Human AML1RUNX1, Drosophila antennapedia, Human AQP4, Human AT1R, Human BAG-1, Human BCL2, Human BiP, Human c-IAP1, Human c-myc, Human eIF4G, Mouse NDST4 L, Human LEF1, Mouse HIF1 alpha, Human n.myc, Mouse Gtx, Human p27kip1, Human PDGF2/c-sis, Human p53, Human Pim-1, Mouse Rbm3, Drosophila reaper, Canine Scamper, Drosophila Ubx, Human UNR, Mouse UtrA, Human VEGF-A, Human XIAP, Salivirus, Cosavirus, Parechovirus, Drosophila hairless, S. cerevisiae TFIID, S. cerevisiae YAP1, Human c-src, Human FGF-1, Simian picomavirus, Turnip crinkle virus, an aptamer to eIF4G, Coxsackievirus B3 (CVB3) or Coxsackievirus A (CVB1/2). In yet another embodiment, the IRES is an IRES sequence of Coxsackievirus B3 (CVB3). In another embodiment, the IRES is an IRES sequence of Encephalomyocarditis virus. In some embodiments, the IRES sequence comprises any one of the sequences set forth in Table 4. In some embodiments, the IRES sequence is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99%, identical to any one of the sequences set forth in Table 4.

In some embodiments, the sequence of interest is a protein coding sequence. The protein coding sequence may encode a protein of eukaryotic or prokaryotic origin. In another embodiment, the protein coding sequence encodes human protein or non-human protein. In some embodiments, the protein coding sequence encodes one or more antibodies. For example, in some embodiments, the protein coding sequence encodes human antibodies. For example, the protein coding sequence may encode a protein selected from hFIX, SP-B, VEGF-A, human methylmalonyl-CoA mutase (hMUT), CFTR, cancer self-antigens, and additional gene editing enzymes like Cpf1, zinc finger nucleases (ZFNs) or transcription activator-like effector nucleases (TALENs). In one embodiment, the protein coding sequence encodes a protein for therapeutic use. In one embodiment, the antibody encoded by the protein coding sequence is a bispecific antibody. In some embodiments, the protein coding region encodes a protein for diagnostic use. In some embodiments, the protein coding region encodes Gaussia luciferase (Gluc), Firefly luciferase (Fluc), enhanced green fluorescent protein (eGFP), human erythropoietin (hEPO), mScarlet fluorescent protein or Cas9 endonuclease (e.g., a reporter sequence).

The sequence of interest may be an antagomir. Antagomirs are RNA-like oligonucleotides that harbor various modifications for RNAse protection and pharmacologic properties, such as enhanced tissue and cellular uptake. They differ from normal RNA by, for example, complete 2′-O-methylation of sugar, phosphorothioate backbone and, for example, a cholesterol-moiety at the 3′-end. Antagomirs may be used to efficiently silence endogenous miRNAs by forming duplexes comprising the antagomir and endogenous miRNA, thereby preventing miRNA-induced gene silencing. An example of antagomir-mediated miRNA silencing is the silencing of miR-122, described in Krutzfeldt et al., “Silencing of microRNAs In Vivo with ‘Antagomirs’,” Nature 438(7068):685-689 (2005), which is hereby incorporated by reference in its entirety). MicroRNAs (“miRNAs” or “mirs”) are a highly conserved class of small RNA molecules that are transcribed from DNA in the genomes of plants and animals, but are not translated into protein. Pre-microRNAs are processed into miRNAs. Processed microRNAs are single stranded ^{˜}17-25 nucleotide RNA molecules that become incorporated into the RNA-induced silencing complex (“RISC”) and have been identified as key regulators of development, cell proliferation, apoptosis, and differentiation. They are believed to play a role in regulation of gene expression by binding to the 3′-untranslated region of specific mRNAs. RISC mediates down-regulation of gene expression through translational inhibition, transcript cleavage, or both. RISC is also implicated in transcriptional silencing in the nucleus of a wide range of eukaryotes.

In some embodiments, the sequence of interest is an aptamer. As used herein, the term “aptamer” refers to a nucleic acid molecule that binds with high affinity and specificity to a target. Nucleic acid aptamers may be single-stranded, partially single-stranded, partially double-stranded, or double-stranded nucleotide sequences. Aptamers include, without limitation, defined sequence segments and sequences comprising nucleotides, ribonucleotides, deoxyribonucleotides, nucleotide analogs, modified nucleotides, and nucleotides comprising backbone modifications, branch points, and non-nucleotide residues, groups, or bridges. Nucleic acid aptamers include partially and fully single-stranded and double-stranded nucleotide molecules and sequences; synthetic RNA, DNA, and chimeric nucleotides; hybrids; duplexes; heteroduplexes; and any ribonucleotide, deoxyribonucleotide, or chimeric counterpart thereof and/or corresponding complementary sequence, promoter, or primer-annealing sequence needed to amplify, transcribe, or replicate all or part of the aptamer molecule or sequence.

The aptamer may comprise a fluorogenic aptamer. Fluorogenic aptamers are well known in the art and include, without limitation, Spinach, Spinach 2, Broccoli, Red-Broccoli, Orange Broccoli, Corn, Mango, Malachite Green, cobalamine-binding aptamer, and derivatives thereof. See, e.g., Autour et al., “Fluorogenic RNA Mango Aptamers for Imaging Small Non-Coding RNAs in Mammalian Cells,” Nature Comm. 9: Article 656 (2018); Jaffrey, S., “RNA-Based Fluorescent Biosensors for Detecting Metabolites In Vitro and in Living Cells,” Adv Pharmacol. 82:187-203 (2018); and Litke et al., “Developing Fluorogenic Riboswitches for Imaging Metabolite Concentration Dynamics in Bacterial Cells,” Methods Enzymol. 572:315-33 (2016), each of which are hereby incorporated by reference in their entirety). In accordance with this embodiment, the fluorogenic aptamer binds to a fluorophore whose fluorescence, absorbance, spectral properties, or quenching properties are increased, decreased, or altered by interaction with the fluorogenic aptamer. Any aptamer-dye complex, some of which are fluorogenic aptamers, may be used. In addition, some aptamers can bind quenchers and some do other things to change the photophysical properties of dyes. In another embodiment, the aptamer binds a target molecule of interest. The target molecule of interest may be any biomaterial or small molecule including, without limitation, proteins, nucleic acids (RNA or DNA), lipids, oligosaccharides, carbohydrates, small molecules, hormones, cytokines, chemokines, cell signaling molecules, metabolites, organic molecules, and metal ions. The target molecule of interest may be one that is associated with a disease state or pathogen infection.

In some embodiments, the sequence of interest comprises a fluorogenic aptamer coupled to an aptamer that binds a target molecule. In accordance with this embodiment, the sequence of interest may be a sensor. In accordance with this embodiment, the fluorogenic aptamer is coupled to an aptamer that binds a target molecule using a transducer stem. Suitable target molecules of interest include, but are not limited to, ADP, adenosine, guanine, GTP, SAM, and streptavidin. As demonstrated in the accompanying Examples, circular aptamer “sensors” can be developed, e.g., against SAM.

In some embodiments, the sequence of interest is an RNA silencing agent (also referred to herein as an “interfering RNA molecule”), such as a small interfering RNA (siRNA), CRISPR RNA (crRNA), a small hairpin RNA (shRNA), a microRNA (miRNA), or a piwi-interacting RNA (piRNA). RNA silencing agents generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. The interfering RNA molecules can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications. The backbone is uncharged, resulting in PNA/DNA or PNA/RNA duplexes that exhibit greater than normal thermal stability. PNAs are not recognized by nucleases or proteases. Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™. has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability. The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo- and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

In some embodiments, the interfering RNA molecule is an siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC). In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length), or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety.

In some embodiments, the sequence of interest is a micro RNA (miRNA). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, the sequence of interest is a CRISPR guide RNA (such as a single guide RNA (sgRNA)). A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a CRISPR RNA (or crRNA). Other gRNAs are a single RNA molecule (single RNA polynucleotide), which can also be called a “single-molecule gRNA,” a “single-guide RNA,” or an “sgRNA.” The terms “guide RNA” and “gRNA” include both double-molecule (i.e., modular) gRNAs and single-molecule gRNAs.

In some embodiments, the sequence of interest comprises a sequence bound by a RNA binding protein (i.e., a RBP). RBPs play key roles in post-transcriptional processes in eukaryotes, such as splicing regulation, mRNA transport and modulation of mRNA translation and decay. In some embodiments, RBPs assemble into different mRNA-protein complexes, which may form messenger ribonucleoprotein complexes (mRNPs). Additional details on RPBs can be found in Gebauer, F., et al. RNA-binding proteins in human genetic disease. Nat Rev Genet 22, 185-198 (2021), which is hereby incorporated by reference in its entirety.

In some embodiments, the sequence of interest comprises a region of non-coding nucleic acids, such as a spacer sequence or a translation regulation motif Translation regulation motifs include, but are not limited to, RNA sequences and/or structures that are commonly located in the untranslated regions of RNA transcripts. Translation regulation motifs may be recognized by regulatory proteins or micro RNAs (miRNAs).

In some embodiments, the sequence of interest encodes a protein, such as an antibody. Unless otherwise specified here within, the terms “antibody” and “antibodies” refers to antigen-binding portions adaptable to be expressed within cells as “intracellular antibodies.” (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

Antibodies may be polyclonal or monoclonal; xenogeneic, allogeneic, or syngeneic; or modified forms thereof (e.g. humanized, chimeric, etc.). Antibodies may also be fully human. Preferably, antibodies bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized”, which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. Humanized antibodies may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

In yet another embodiment, the sequence of interest encodes an intrabody, or an antigen binding fragment thereof. In another embodiment, the intrabody, or antigen binding fragment thereof, is a murine, chimeric, humanized, composite, or human intrabody, or antigen binding fragment thereof. In another embodiment, the intrabody, or antigen binding fragment thereof, is detectably labeled, comprises an effector domain, comprises an Fc domain, and/or is selected from the group consisting of Fv, Fav, F(ab′)2, Fab′, dsFv, scFv, sc(Fv)2, and diabody fragments.

The sequence of interest can range, without limitation, from 10 bp to 10 Kbp. In some embodiments, the sequence of interest can be at least 10 bp, at least 15 bp, at least 20 bp, at least 25 bp, at least 30 bp, at least 35 bp, at least 40 bp, at least 45 bp, at least 50 bp, at least 55 bp, at least 60 bp, at least 65 bp, at least 70 bp, at least 75 bp, at least 80 bp, at least 85 bp, at least 90 bp, at least 95 bp, at least 100 bp, at least 105 bp, at least 110 bp, at least 115 bp, at least 120 bp, at least 125 bp, at least 130 bp, at least 135 bp, at least 140 bp, at least 145 bp, at least 150 bp, at least 155 bp, at least 160 bp, at least 165 bp, at least 170 bp, at least 175 bp, at least 180 bp, at least 185 bp, at least 190 bp, at least 195 bp, at least 200 bp, at least 205 bp, at least 210 bp, at least 215 bp, at least 220 bp, at least 225 bp, at least 230 bp, at least 235 bp, at least 240 bp, at least 245 bp, at least 250 bp, at least 255 bp, at least 260 bp, at least 265 bp, at least 270 bp, at least 275 bp, at least 280 bp, at least 285 bp, at least 290 bp, at least 295 bp, at least 300 bp, at least 305 bp, at least 310 bp, at least 315 bp, at least 320 bp, at least 325 bp, at least 330 bp, at least 335 bp, at least 340 bp, at least 345 bp, at least 350 bp, at least 355 bp, at least 360 bp, at least 365 bp, at least 370 bp, at least 375 bp, at least 380 bp, at least 385 bp, at least 390 bp, at least 395 bp, at least 400 bp, at least 405 bp, at least 410 bp, at least 415 bp, at least 420 bp, at least 425 bp, at least 430 bp, at least 435 bp, at least 440 bp, at least 445 bp, at least 450 bp, at least 455 bp, at least 460 bp, at least 465 bp, at least 470 bp, at least 475 bp, at least 480 bp, at least 485 bp, at least 490 bp, at least 495 bp, at least 500 bp, at least 505 bp, at least 510 bp, at least 515 bp, at least 520 bp, at least 525 bp, at least 530 bp, at least 535 bp, at least 540 bp, at least 545 bp, at least 550 bp, at least 555 bp, at least 560 bp, at least 565 bp, at least 570 bp, at least 575 bp, at least 580 bp, at least 585 bp, at least 590 bp, at least 595 bp, at least 600 bp, at least 605 bp, at least 610 bp, at least 615 bp, at least 620 bp, at least 625 bp, at least 630 bp, at least 635 bp, at least 640 bp, at least 645 bp, at least 650 bp, at least 655 bp, at least 660 bp, at least 665 bp, at least 670 bp, at least 675 bp, at least 680 bp, at least 685 bp, at least 690 bp, at least 695 bp, at least 700 bp, at least 705 bp, at least 710 bp, at least 715 bp, at least 720 bp, at least 725 bp, at least 730 bp, at least 735 bp, at least 740 bp, at least 745 bp, at least 750 bp, at least 755 bp, at least 760 bp, at least 765 bp, at least 770 bp, at least 775 bp, at least 780 bp, at least 785 bp, at least 790 bp, at least 795 bp, at least 800 bp, at least 805 bp, at least 810 bp, at least 815 bp, at least 820 bp, at least 825 bp, at least 830 bp, at least 835 bp, at least 840 bp, at least 845 bp, at least 850 bp, at least 855 bp, at least 860 bp, at least 865 bp, at least 870 bp, at least 875 bp, at least 880 bp, at least 885 bp, at least 890 bp, at least 895 bp, at least 900 bp, at least 905 bp, at least 910 bp, at least 915 bp, at least 920 bp, at least 925 bp, at least 930 bp, at least 935 bp, at least 940 bp, at least 945 bp, at least 950 bp, at least 955 bp, at least 960 bp, at least 965 bp, at least 970 bp, at least 975 bp, at least 980 bp, at least 985 bp, at least 990 bp, at least 995 bp, at least 1000 bp, at least 1025 bp, at least 1050 bp, at least 1075 bp, at least 1100 bp, at least 1125 bp, at least 1150 bp, at least 1175 bp, at least 1200 bp, at least 1225 bp, at least 1250 bp, at least 1275 bp, at least 1300 bp, at least 1325 bp, at least 1350 bp, at least 1375 bp, at least 1400 bp, at least 1425 bp, at least 1450 bp, at least 1475 bp, at least 1500 bp, at least 1525 bp, at least 1550 bp, at least 1575 bp, at least 1600 bp, at least 1625 bp, at least 1650 bp, at least 1675 bp, at least 1700 bp, at least 1725 bp, at least 1750 bp, at least 1775 bp, at least 1800 bp, at least 1825 bp, at least 1850 bp, at least 1875 bp, at least 1900 bp, at least 1925 bp, at least 1950 bp, at least 1975 bp, at least 2000 bp, at least 2025 bp, at least 2050 bp, at least 2075 bp, at least 2100 bp, at least 2125 bp, at least 2150 bp, at least 2175 bp, at least 2200 bp, at least 2225 bp, at least 2250 bp, at least 2275 bp, at least 2300 bp, at least 2325 bp, at least 2350 bp, at least 2375 bp, at least 2400 bp, at least 2425 bp, at least 2450 bp, at least 2475 bp, at least 2500 bp, at least 2525 bp, at least 2550 bp, at least 2575 bp, at least 2600 bp, at least 2625 bp, at least 2650 bp, at least 2675 bp, at least 2700 bp, at least 2725 bp, at least 2750 bp, at least 2775 bp, at least 2800 bp, at least 2825 bp, at least 2850 bp, at least 2875 bp, at least 2900 bp, at least 2925 bp, at least 2950 bp, at least 2975 bp, at least 3000 bp, at least 3025 bp, at least 3050 bp, at least 3075 bp, at least 3100 bp, at least 3125 bp, at least 3150 bp, at least 3175 bp, at least 3200 bp, at least 3225 bp, at least 3250 bp, at least 3275 bp, at least 3300 bp, at least 3325 bp, at least 3350 bp, at least 3375 bp, at least 3400 bp, at least 3425 bp, at least 3450 bp, at least 3475 bp, at least 3500 bp, at least 3525 bp, at least 3550 bp, at least 3575 bp, at least 3600 bp, at least 3625 bp, at least 3650 bp, at least 3675 bp, at least 3700 bp, at least 3725 bp, at least 3750 bp, at least 3775 bp, at least 3800 bp, at least 3825 bp, at least 3850 bp, at least 3875 bp, at least 3900 bp, at least 3925 bp, at least 3950 bp, at least 3975 bp, at least 4000 bp, at least 4025 bp, at least 4050 bp, at least 4075 bp, at least 4100 bp, at least 4125 bp, at least 4150 bp, at least 4175 bp, at least 4200 bp, at least 4225 bp, at least 4250 bp, at least 4275 bp, at least 4300 bp, at least 4325 bp, at least 4350 bp, at least 4375 bp, at least 4400 bp, at least 4425 bp, at least 4450 bp, at least 4475 bp, at least 4500 bp, at least 4525 bp, at least 4550 bp, at least 4575 bp, at least 4600 bp, at least 4625 bp, at least 4650 bp, at least 4675 bp, at least 4700 bp, at least 4725 bp, at least 4750 bp, at least 4775 bp, at least 4800 bp, at least 4825 bp, at least 4850 bp, at least 4875 bp, at least 4900 bp, at least 4925 bp, at least 4950 bp, at least 4975 bp, at least 5000 bp, at least 5025 bp, at least 5050 bp, at least 5075 bp, at least 5100 bp, at least 5125 bp, at least 5150 bp, at least 5175 bp, at least 5200 bp, at least 5225 bp, at least 5250 bp, at least 5275 bp, at least 5300 bp, at least 5325 bp, at least 5350 bp, at least 5375 bp, at least 5400 bp, at least 5425 bp, at least 5450 bp, at least 5475 bp, at least 5500 bp, at least 5525 bp, at least 5550 bp, at least 5575 bp, at least 5600 bp, at least 5625 bp, at least 5650 bp, at least 5675 bp, at least 5700 bp, at least 5725 bp, at least 5750 bp, at least 5775 bp, at least 5800 bp, at least 5825 bp, at least 5850 bp, at least 5875 bp, at least 5900 bp, at least 5925 bp, at least 5950 bp, at least 5975 bp, at least 6000 bp, at least 6025 bp, at least 6050 bp, at least 6075 bp, at least 6100 bp, at least 6125 bp, at least 6150 bp, at least 6175 bp, at least 6200 bp, at least 6225 bp, at least 6250 bp, at least 6275 bp, at least 6300 bp, at least 6325 bp, at least 6350 bp, at least 6375 bp, at least 6400 bp, at least 6425 bp, at least 6450 bp, at least 6475 bp, at least 6500 bp, at least 6525 bp, at least 6550 bp, at least 6575 bp, at least 6600 bp, at least 6625 bp, at least 6650 bp, at least 6675 bp, at least 6700 bp, at least 6725 bp, at least 6750 bp, at least 6775 bp, at least 6800 bp, at least 6825 bp, at least 6850 bp, at least 6875 bp, at least 6900 bp, at least 6925 bp, at least 6950 bp, at least 6975 bp, at least 7000 bp, at least 7025 bp, at least 7050 bp, at least 7075 bp, at least 7100 bp, at least 7125 bp, at least 7150 bp, at least 7175 bp, at least 7200 bp, at least 7225 bp, at least 7250 bp, at least 7275 bp, at least 7300 bp, at least 7325 bp, at least 7350 bp, at least 7375 bp, at least 7400 bp, at least 7425 bp, at least 7450 bp, at least 7475 bp, at least 7500 bp, at least 7525 bp, at least 7550 bp, at least 7575 bp, at least 7600 bp, at least 7625 bp, at least 7650 bp, at least 7675 bp, at least 7700 bp, at least 7725 bp, at least 7750 bp, at least 7775 bp, at least 7800 bp, at least 7825 bp, at least 7850 bp, at least 7875 bp, at least 7900 bp, at least 7925 bp, at least 7950 bp, at least 7975 bp, at least 8000 bp, at least 8025 bp, at least 8050 bp, at least 8075 bp, at least 8100 bp, at least 8125 bp, at least 8150 bp, at least 8175 bp, at least 8200 bp, at least 8225 bp, at least 8250 bp, at least 8275 bp, at least 8300 bp, at least 8325 bp, at least 8350 bp, at least 8375 bp, at least 8400 bp, at least 8425 bp, at least 8450 bp, at least 8475 bp, at least 8500 bp, at least 8525 bp, at least 8550 bp, at least 8575 bp, at least 8600 bp, at least 8625 bp, at least 8650 bp, at least 8675 bp, at least 8700 bp, at least 8725 bp, at least 8750 bp, at least 8775 bp, at least 8800 bp, at least 8825 bp, at least 8850 bp, at least 8875 bp, at least 8900 bp, at least 8925 bp, at least 8950 bp, at least 8975 bp, at least 9000 bp, at least 9025 bp, at least 9050 bp, at least 9075 bp, at least 9100 bp, at least 9125 bp, at least 9150 bp, at least 9175 bp, at least 9200 bp, at least 9225 bp, at least 9250 bp, at least 9275 bp, at least 9300 bp, at least 9325 bp, at least 9350 bp, at least 9375 bp, at least 9400 bp, at least 9425 bp, at least 9450 bp, at least 9475 bp, at least 9500 bp, at least 9525 bp, at least 9550 bp, at least 9575 bp, at least 9600 bp, at least 9625 bp, at least 9650 bp, at least 9675 bp, at least 9700 bp, at least 9725 bp, at least 9750 bp, at least 9775 bp, at least 9800 bp, at least 9825 bp, at least 9850 bp, at least 9875 bp, at least 9900 bp, at least 9925 bp, at least 9950 bp, at least 9975 bp, at least 10000 bp. For example, in some embodiments, the sequence of interest may be between 200 bp and 10 kbp, between 300 bp and 10 kbp between 400 bp and 10 kbp, between 500 bp and 10 kbp, 600 bp and 10 kbp, between 700 bp and 10 kbp between 800 bp and 10 kbp, between 900 bp and 10 kbp, 1 kbp and 10 kbp, between 2 kbp and 10 kbp between 3 kbp and 10 kbp, 4 kbp and 10 kbp, between 5 kbp and 10 kbp between 6 kbp and 10 kbp, 7 kbp and 10 kbp, between 8 kbp and 10 kbp or between 9 kbp and 10 kbp.

In some embodiments, the sequence of interest is no more than 300 bp, 305 bp, 310 bp, 315 bp, 320 bp, 325 bp, 330 bp, 335 bp, 340 bp, 345 bp, 350 bp, 355 bp, 360 bp, 365 bp, 370 bp, 375 bp, 380 bp, 385 bp, 390 bp, 395 bp, 400 bp, 405 bp, 410 bp, 415 bp, 420 bp, 425 bp, 430 bp, 435 bp, 440 bp, 445 bp, 450 bp, 455 bp, 460 bp, 465 bp, 470 bp, 475 bp, 480 bp, 485 bp, 490 bp, 495 bp, 500 bp, 505 bp, 510 bp, 515 bp, 520 bp, 525 bp, 530 bp, 535 bp, 540 bp, 545 bp, 550 bp, 555 bp, 560 bp, 565 bp, 570 bp, 575 bp, 580 bp, 585 bp, 590 bp, 595 bp, 600 bp, 605 bp, 610 bp, 615 bp, 620 bp, 625 bp, 630 bp, 635 bp, 640 bp, 645 bp, 650 bp, 655 bp, 660 bp, 665 bp, 670 bp, 675 bp, 680 bp, 685 bp, 690 bp, 695 bp, 700 bp, 705 bp, 710 bp, 715 bp, 720 bp, 725 bp, 730 bp, 735 bp, 740 bp, 745 bp, 750 bp, 755 bp, 760 bp, 765 bp, 770 bp, 775 bp, 780 bp, 785 bp, 790 bp, 795 bp, 800 bp, 805 bp, 810 bp, 815 bp, 820 bp, 825 bp, 830 bp, 835 bp, 840 bp, 845 bp, 850 bp, 855 bp, 860 bp, 865 bp, 870 bp, 875 bp, 880 bp, 885 bp, 890 bp, 895 bp, 900 bp, 905 bp, 910 bp, 915 bp, 920 bp, 925 bp, 930 bp, 935 bp, 940 bp, 945 bp, 950 bp, 955 bp, 960 bp, 965 bp, 970 bp, 975 bp, 980 bp, 985 bp, 990 bp, 995 bp, 1000 bp, 1025 bp, 1050 bp, 1075 bp, 1100 bp, 1125 bp, 1150 bp, 1175 bp, 1200 bp, 1225 bp, 1250 bp, 1275 bp, 1300 bp, 1325 bp, 1350 bp, 1375 bp, 1400 bp, 1425 bp, 1450 bp, 1475 bp, 1500 bp, 1525 bp, 1550 bp, 1575 bp, 1600 bp, 1625 bp, 1650 bp, 1675 bp, 1700 bp, 1725 bp, 1750 bp, 1775 bp, 1800 bp, 1825 bp, 1850 bp, 1875 bp, 1900 bp, 1925 bp, 1950 bp, 1975 bp, 2000 bp, 2025 bp, 2050 bp, 2075 bp, 2100 bp, 2125 bp, 2150 bp, 2175 bp, 2200 bp, 2225 bp, 2250 bp, 2275 bp, 2300 bp, 2325 bp, 2350 bp, 2375 bp, 2400 bp, 2425 bp, 2450 bp, 2475 bp, 2500 bp, 2525 bp, 2550 bp, 2575 bp, 2600 bp, 2625 bp, 2650 bp, 2675 bp, 2700 bp, 2725 bp, 2750 bp, 2775 bp, 2800 bp, 2825 bp, 2850 bp, 2875 bp, 2900 bp, 2925 bp, 2950 bp, 2975 bp, 3000 bp, 3025 bp, 3050 bp, 3075 bp, 3100 bp, 3125 bp, 3150 bp, 3175 bp, 3200 bp, 3225 bp, 3250 bp, 3275 bp, 3300 bp, 3325 bp, 3350 bp, 3375 bp, 3400 bp, 3425 bp, 3450 bp, 3475 bp, 3500 bp, 3525 bp, 3550 bp, 3575 bp, 3600 bp, 3625 bp, 3650 bp, 3675 bp, 3700 bp, 3725 bp, 3750 bp, 3775 bp, 3800 bp, 3825 bp, 3850 bp, 3875 bp, 3900 bp, 3925 bp, 3950 bp, 3975 bp, 4000 bp, 4025 bp, 4050 bp, 4075 bp, 4100 bp, 4125 bp, 4150 bp, 4175 bp, 4200 bp, 4225 bp, 4250 bp, 4275 bp, 4300 bp, 4325 bp, 4350 bp, 4375 bp, 4400 bp, 4425 bp, 4450 bp, 4475 bp, 4500 bp, 4525 bp, 4550 bp, 4575 bp, 4600 bp, 4625 bp, 4650 bp, 4675 bp, 4700 bp, 4725 bp, 4750 bp, 4775 bp, 4800 bp, 4825 bp, 4850 bp, 4875 bp, 4900 bp, 4925 bp, 4950 bp, 4975 bp, 5000 bp, 5025 bp, 5050 bp, 5075 bp, 5100 bp, 5125 bp, 5150 bp, 5175 bp, 5200 bp, 5225 bp, 5250 bp, 5275 bp, 5300 bp, 5325 bp, 5350 bp, 5375 bp, 5400 bp, 5425 bp, 5450 bp, 5475 bp, 5500 bp, 5525 bp, 5550 bp, 5575 bp, 5600 bp, 5625 bp, 5650 bp, 5675 bp, 5700 bp, 5725 bp, 5750 bp, 5775 bp, 5800 bp, 5825 bp, 5850 bp, 5875 bp, 5900 bp, 5925 bp, 5950 bp, 5975 bp, 6000 bp, 6025 bp, 6050 bp, 6075 bp, 6100 bp, 6125 bp, 6150 bp, 6175 bp, 6200 bp, 6225 bp, 6250 bp, 6275 bp, 6300 bp, 6325 bp, 6350 bp, 6375 bp, 6400 bp, 6425 bp, 6450 bp, 6475 bp, 6500 bp, 6525 bp, 6550 bp, 6575 bp, 6600 bp, 6625 bp, 6650 bp, 6675 bp, 6700 bp, 6725 bp, 6750 bp, 6775 bp, 6800 bp, 6825 bp, 6850 bp, 6875 bp, 6900 bp, 6925 bp, 6950 bp, 6975 bp, 7000 bp, 7025 bp, 7050 bp, 7075 bp, 7100 bp, 7125 bp, 7150 bp, 7175 bp, 7200 bp, 7225 bp, 7250 bp, 7275 bp, 7300 bp, 7325 bp, 7350 bp, 7375 bp, 7400 bp, 7425 bp, 7450 bp, 7475 bp, 7500 bp, 7525 bp, 7550 bp, 7575 bp, 7600 bp, 7625 bp, 7650 bp, 7675 bp, 7700 bp, 7725 bp, 7750 bp, 7775 bp, 7800 bp, 7825 bp, 7850 bp, 7875 bp, 7900 bp, 7925 bp, 7950 bp, 7975 bp, 8000 bp, 8025 bp, 8050 bp, 8075 bp, 8100 bp, 8125 bp, 8150 bp, 8175 bp, 8200 bp, 8225 bp, 8250 bp, 8275 bp, 8300 bp, 8325 bp, 8350 bp, 8375 bp, 8400 bp, 8425 bp, 8450 bp, 8475 bp, 8500 bp, 8525 bp, 8550 bp, 8575 bp, 8600 bp, 8625 bp, 8650 bp, 8675 bp, 8700 bp, 8725 bp, 8750 bp, 8775 bp, 8800 bp, 8825 bp, 8850 bp, 8875 bp, 8900 bp, 8925 bp, 8950 bp, 8975 bp, 9000 bp, 9025 bp, 9050 bp, 9075 bp, 9100 bp, 9125 bp, 9150 bp, 9175 bp, 9200 bp, 9225 bp, 9250 bp, 9275 bp, 9300 bp, 9325 bp, 9350 bp, 9375 bp, 9400 bp, 9425 bp, 9450 bp, 9475 bp, 9500 bp, 9525 bp, 9550 bp, 9575 bp, 9600 bp, 9625 bp, 9650 bp, 9675 bp, 9700 bp, 9725 bp, 9750 bp, 9775 bp, 9800 bp, 9825 bp, 9850 bp, 9875 bp, 9900 bp, 9925 bp, 9950 bp, 9975 bp, or 10000 bp in length.

In some embodiments, a binding site is present in the nucleic acid molecule; for example, the binding site can bind a primer for reverse transcription, a RNA polymerase, a transcription factor, and/or combinations thereof.

In some embodiments, the nucleic acid molecule further comprises a promoter sequence.

In some embodiments, the promoter is located between the upstream cleavage site and the central ribozyme catalytic core. In some embodiments, the promoter is located between the central ribozyme catalytic core and the sequence of interest. In one embodiment, the nucleic acid molecule comprises an RNA polymerase promoter. The RNA polymerase promoter may be, for example, a T7 virus RNA polymerase promoter, a T6 virus RNA polymerase promoter, a SP6 virus RNA polymerase promoter, a T3 virus RNA polymerase promoter, or a T4 virus RNA polymerase promoter.

The promoter may be a constitutively active promoter (i.e., a promoter that is constitutively in an active or “on” state), an inducible promoter (i.e., a promoter whose state, active or inactive state, is controlled by an external stimulus, e.g., the presence of a particular temperature, compound, or protein), a spatially restricted promoter (i.e., transcriptional control element, enhancer, etc.), a tissue specific promoter, a cell type specific promoter, or a temporally restricted promoter (i.e., the promoter is in the “on” state or “off” state during specific stages of a biological process).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., RNA Polymerase I, RNA Polymerase II, RNA Polymerase III). Exemplary promoters include, but are not limited to a SV40 early promoter, a mouse mammary tumor virus long terminal repeat (“LTR”) promoter; an adenovirus major late promoter (“Ad MLP”); a herpes simplex virus (“HSV”) promoter, a cytomegalovirus (“CMV”) promoter such as the CMV immediate early promoter region (“CMVIE”), a rous sarcoma virus (“RSV”) promoter, a human U6 small nuclear promoter (“U6”) (Miyagishi et al., “U6 promoter-driven siRNAs with four uridine 3′ overhangs efficiently suppress targeted gene expression in mammalian cells,” Nature Biotechnology 20:497-500 (2002), which is hereby incorporated by reference in its entirety), an enhanced U6 promoter (e.g., Xia et al., “An enhanced U6 promoter for synthesis of short hairpin RNA,” Nucleic Acids Res. 31(17):e100 (2003), which is hereby incorporated by reference in its entirety), a human H1 promoter (“H1”), and the like.

Examples of inducible promoters include, but are not limited to, T7 RNA polymerase promoters, T3 RNA polymerase promoters, isopropyl-beta-D-thiogalactopyranoside (IPTG)-regulated promoters, lactose induced promoters, heat shock promoters, tetracycline-regulated promoters, steroid-regulated promoters, metal-regulated promoters, etc. Inducible promoters can therefore be regulated by molecules including, but not limited to, doxycycline, RNA polymerase, e.g., T7 RNA polymerase, etc.

In some embodiments, the promoter is a prokaryotic promoter selected from the group consisting of T7, T3, SP6 RNA polymerase, and derivatives thereof. Additional suitable prokaryotic promoters include, without limitation, T71ac, araBAD, trp, lac, Ptac, and pL promoters.

In another embodiment, the promoter is a eukaryotic RNA polymerase I promoter, RNA polymerase III promoter, or a derivative thereof. Exemplary RNA polymerase II promoters include, without limitation, cytomegalovirus (“CMV”), phosphoglycerate kinase-1 (“PGK-1”), and elongation factor 1α (“EF1α”) promoters. In yet another embodiment, the promoter is a eukaryotic RNA polymerase III promoter selected from the group consisting of U6, H1, 56, 7SK, and derivatives thereof. The RNA Polymerase promoter may be mammalian. Suitable mammalian promoters include, without limitation, human, murine, bovine, canine, feline, ovine, porcine, ursine, and simian promoters. In one embodiment, the RNA polymerase promoter sequence is a human promoter.

Nucleic acid molecules can be assessed using in vitro transcription (IVT) according to standard protocols. For example, once the constructs are assembled, PCR can be conducted with an upstream primer containing a RNA polymerase promoter to amplify the nucleic acid molecule and provide an IVT template. IVT is then performed using an appropriate RNA polymerase. Many suitable reverse transcriptases/RNA polymerases are available commercially, such as T7, T3, and SP6, to name but a few. Typically, the IVT reaction is conducted for at least 1 hour or can be allowed to reach equilibrium. The resulting RNA fragments can be assessed on denaturing agarose or acrylamide gels, as well as on non-denaturing gels, with aptamers that bind a fluor, or via qRTPCR.

In some embodiments, the nucleic acid molecule is about 500 to about 10,000 nucleotides. In some embodiments, the nucleic acid molecule is at least 500 nucleotides, at least 550 nucleotides, at least 600 nucleotides, at least 650 nucleotides, at least 700 nucleotides, at least 750 nucleotides, at least 800 nucleotides, at least 850 nucleotides, at least 900 nucleotides, at least 950 nucleotides, at least 1000 nucleotides, at least 1050 nucleotides, at least 1100 nucleotides, at least 1150 nucleotides, at least 1200 nucleotides, at least 1250 nucleotides, at least 1300 nucleotides, at least 1350 nucleotides, at least 1400 nucleotides, at least 1450 nucleotides, at least 1500 nucleotides, at least 1550 nucleotides, at least 1600 nucleotides, at least 1650 nucleotides, at least 1700 nucleotides, at least 1750 nucleotides, at least 1800 nucleotides, at least 1850 nucleotides, at least 1900 nucleotides, at least 1950 nucleotides, at least 2000 nucleotides, at least 2050 nucleotides, at least 2100 nucleotides, at least 2150 nucleotides, at least 2200 nucleotides, at least 2250 nucleotides, at least 2300 nucleotides, at least 2350 nucleotides, at least 2400 nucleotides, at least 2450 nucleotides, at least 2500 nucleotides, at least 2550 nucleotides, at least 2600 nucleotides, at least 2650 nucleotides, at least 2700 nucleotides, at least 2750 nucleotides, at least 2800 nucleotides, at least 2850 nucleotides, at least 2900 nucleotides, at least 2950 nucleotides, at least 3000 nucleotides, at least 3050 nucleotides, at least 3100 nucleotides, at least 3150 nucleotides, at least 3200 nucleotides, at least 3250 nucleotides, at least 3300 nucleotides, at least 3350 nucleotides, at least 3400 nucleotides, at least 3450 nucleotides, at least 3500 nucleotides, at least 3550 nucleotides, at least 3600 nucleotides, at least 3650 nucleotides, at least 3700 nucleotides, at least 3750 nucleotides, at least 3800 nucleotides, at least 3850 nucleotides, at least 3900 nucleotides, at least 3950 nucleotides, at least 4000 nucleotides, at least 4050 nucleotides, at least 4100 nucleotides, at least 4150 nucleotides, at least 4200 nucleotides, at least 4250 nucleotides, at least 4300 nucleotides, at least 4350 nucleotides, at least 4400 nucleotides, at least 4450 nucleotides, at least 4500 nucleotides, at least 4550 nucleotides, at least 4600 nucleotides, at least 4650 nucleotides, at least 4700 nucleotides, at least 4750 nucleotides, at least 4800 nucleotides, at least 4850 nucleotides, at least 4900 nucleotides, at least 4950 nucleotides, or at least 5000 nucleotides, In some embodiments, the nucleic acid molecule is no more than 500 bp, 505 bp, 510 bp, 515 bp, 520 bp, 525 bp, 530 bp, 535 bp, 540 bp, 545 bp, 550 bp, 555 bp, 560 bp, 565 bp, 570 bp, 575 bp, 580 bp, 585 bp, 590 bp, 595 bp, 600 bp, 605 bp, 610 bp, 615 bp, 620 bp, 625 bp, 630 bp, 635 bp, 640 bp, 645 bp, 650 bp, 655 bp, 660 bp, 665 bp, 670 bp, 675 bp, 680 bp, 685 bp, 690 bp, 695 bp, 700 bp, 705 bp, 710 bp, 715 bp, 720 bp, 725 bp, 730 bp, 735 bp, 740 bp, 745 bp, 750 bp, 755 bp, 760 bp, 765 bp, 770 bp, 775 bp, 780 bp, 785 bp, 790 bp, 795 bp, 800 bp, 805 bp, 810 bp, 815 bp, 820 bp, 825 bp, 830 bp, 835 bp, 840 bp, 845 bp, 850 bp, 855 bp, 860 bp, 865 bp, 870 bp, 875 bp, 880 bp, 885 bp, 890 bp, 895 bp, 900 bp, 905 bp, 910 bp, 915 bp, 920 bp, 925 bp, 930 bp, 935 bp, 940 bp, 945 bp, 950 bp, 955 bp, 960 bp, 965 bp, 970 bp, 975 bp, 980 bp, 985 bp, 990 bp, 995 bp, 1000 bp, 1025 bp, 1050 bp, 1075 bp, 1100 bp, 1125 bp, 1150 bp, 1175 bp, 1200 bp, 1225 bp, 1250 bp, 1275 bp, 1300 bp, 1325 bp, 1350 bp, 1375 bp, 1400 bp, 1425 bp, 1450 bp, 1475 bp, 1500 bp, 1525 bp, 1550 bp, 1575 bp, 1600 bp, 1625 bp, 1650 bp, 1675 bp, 1700 bp, 1725 bp, 1750 bp, 1775 bp, 1800 bp, 1825 bp, 1850 bp, 1875 bp, 1900 bp, 1925 bp, 1950 bp, 1975 bp, 2000 bp, 2025 bp, 2050 bp, 2075 bp, 2100 bp, 2125 bp, 2150 bp, 2175 bp, 2200 bp, 2225 bp, 2250 bp, 2275 bp, 2300 bp, 2325 bp, 2350 bp, 2375 bp, 2400 bp, 2425 bp, 2450 bp, 2475 bp, 2500 bp, 2525 bp, 2550 bp, 2575 bp, 2600 bp, 2625 bp, 2650 bp, 2675 bp, 2700 bp, 2725 bp, 2750 bp, 2775 bp, 2800 bp, 2825 bp, 2850 bp, 2875 bp, 2900 bp, 2925 bp, 2950 bp, 2975 bp, 3000 bp, 3025 bp, 3050 bp, 3075 bp, 3100 bp, 3125 bp, 3150 bp, 3175 bp, 3200 bp, 3225 bp, 3250 bp, 3275 bp, 3300 bp, 3325 bp, 3350 bp, 3375 bp, 3400 bp, 3425 bp, 3450 bp, 3475 bp, 3500 bp, 3525 bp, 3550 bp, 3575 bp, 3600 bp, 3625 bp, 3650 bp, 3675 bp, 3700 bp, 3725 bp, 3750 bp, 3775 bp, 3800 bp, 3825 bp, 3850 bp, 3875 bp, 3900 bp, 3925 bp, 3950 bp, 3975 bp, 4000 bp, 4025 bp, 4050 bp, 4075 bp, 4100 bp, 4125 bp, 4150 bp, 4175 bp, 4200 bp, 4225 bp, 4250 bp, 4275 bp, 4300 bp, 4325 bp, 4350 bp, 4375 bp, 4400 bp, 4425 bp, 4450 bp, 4475 bp, 4500 bp, 4525 bp, 4550 bp, 4575 bp, 4600 bp, 4625 bp, 4650 bp, 4675 bp, 4700 bp, 4725 bp, 4750 bp, 4775 bp, 4800 bp, 4825 bp, 4850 bp, 4875 bp, 4900 bp, 4925 bp, 4950 bp, 4975 bp, 5000 bp, 5025 bp, 5050 bp, 5075 bp, 5100 bp, 5125 bp, 5150 bp, 5175 bp, 5200 bp, 5225 bp, 5250 bp, 5275 bp, 5300 bp, 5325 bp, 5350 bp, 5375 bp, 5400 bp, 5425 bp, 5450 bp, 5475 bp, 5500 bp, 5525 bp, 5550 bp, 5575 bp, 5600 bp, 5625 bp, 5650 bp, 5675 bp, 5700 bp, 5725 bp, 5750 bp, 5775 bp, 5800 bp, 5825 bp, 5850 bp, 5875 bp, 5900 bp, 5925 bp, 5950 bp, 5975 bp, 6000 bp, 6025 bp, 6050 bp, 6075 bp, 6100 bp, 6125 bp, 6150 bp, 6175 bp, 6200 bp, 6225 bp, 6250 bp, 6275 bp, 6300 bp, 6325 bp, 6350 bp, 6375 bp, 6400 bp, 6425 bp, 6450 bp, 6475 bp, 6500 bp, 6525 bp, 6550 bp, 6575 bp, 6600 bp, 6625 bp, 6650 bp, 6675 bp, 6700 bp, 6725 bp, 6750 bp, 6775 bp, 6800 bp, 6825 bp, 6850 bp, 6875 bp, 6900 bp, 6925 bp, 6950 bp, 6975 bp, 7000 bp, 7025 bp, 7050 bp, 7075 bp, 7100 bp, 7125 bp, 7150 bp, 7175 bp, 7200 bp, 7225 bp, 7250 bp, 7275 bp, 7300 bp, 7325 bp, 7350 bp, 7375 bp, 7400 bp, 7425 bp, 7450 bp, 7475 bp, 7500 bp, 7525 bp, 7550 bp, 7575 bp, 7600 bp, 7625 bp, 7650 bp, 7675 bp, 7700 bp, 7725 bp, 7750 bp, 7775 bp, 7800 bp, 7825 bp, 7850 bp, 7875 bp, 7900 bp, 7925 bp, 7950 bp, 7975 bp, 8000 bp, 8025 bp, 8050 bp, 8075 bp, 8100 bp, 8125 bp, 8150 bp, 8175 bp, 8200 bp, 8225 bp, 8250 bp, 8275 bp, 8300 bp, 8325 bp, 8350 bp, 8375 bp, 8400 bp, 8425 bp, 8450 bp, 8475 bp, 8500 bp, 8525 bp, 8550 bp, 8575 bp, 8600 bp, 8625 bp, 8650 bp, 8675 bp, 8700 bp, 8725 bp, 8750 bp, 8775 bp, 8800 bp, 8825 bp, 8850 bp, 8875 bp, 8900 bp, 8925 bp, 8950 bp, 8975 bp, 9000 bp, 9025 bp, 9050 bp, 9075 bp, 9100 bp, 9125 bp, 9150 bp, 9175 bp, 9200 bp, 9225 bp, 9250 bp, 9275 bp, 9300 bp, 9325 bp, 9350 bp, 9375 bp, 9400 bp, 9425 bp, 9450 bp, 9475 bp, 9500 bp, 9525 bp, 9550 bp, 9575 bp, 9600 bp, 9625 bp, 9650 bp, 9675 bp, 9700 bp, 9725 bp, 9750 bp, 9775 bp, 9800 bp, 9825 bp, 9850 bp, 9875 bp, 9900 bp, 9925 bp, 9950 bp, 9975 bp, or 10000 bp in length.

In some embodiments, the circular nucleic acid molecule is less than 10,000, 9,000, 8,000, 7,000, 6,000, 5,000 or 4,000 nucleotides in size. In some embodiments, the circular nucleic acid molecule is at least 25 nucleotides, at least 50 nucleotides, at least 75 nucleotides, at least 100 nucleotides, at least 125 nucleotides, at least 150 nucleotides, at least 175 nucleotides, at least 200 nucleotides, at least 225 nucleotides, at least 250 nucleotides, at least 275 nucleotides, at least 300 nucleotides, at least 325 nucleotides, at least 350 nucleotides, at least 375 nucleotides, at least 400 nucleotides, at least 425 nucleotides, at least 450 nucleotides, at least 475 nucleotides, at least 500 nucleotides, at least 525 nucleotides, at least 550 nucleotides, at least 575 nucleotides, at least 600 nucleotides, at least 625 nucleotides, at least 650 nucleotides, at least 675 nucleotides, at least 700 nucleotides, at least 725 nucleotides, at least 750 nucleotides, at least 775 nucleotides, at least 800 nucleotides, at least 825 nucleotides, at least 850 nucleotides, at least 875 nucleotides, at least 900 nucleotides, at least 925 nucleotides, at least 950 nucleotides, at least 975 nucleotides, at least 1000 nucleotides, at least 1025 nucleotides, at least 1050 nucleotides, at least 1075 nucleotides, at least 1100 nucleotides, at least 1125 nucleotides, at least 1150 nucleotides, at least 1175 nucleotides, at least 1200 nucleotides, at least 1225 nucleotides, at least 1250 nucleotides, at least 1275 nucleotides, at least 1300 nucleotides, at least 1325 nucleotides, at least 1350 nucleotides, at least 1375 nucleotides, at least 1400 nucleotides, at least 1425 nucleotides, at least 1450 nucleotides, at least 1475 nucleotides, at least 1500 nucleotides, at least 1525 nucleotides, at least 1550 nucleotides, at least 1575 nucleotides, at least 1600 nucleotides, at least 1625 nucleotides, at least 1650 nucleotides, at least 1675 nucleotides, at least 1700 nucleotides, at least 1725 nucleotides, at least 1750 nucleotides, at least 1775 nucleotides, at least 1800 nucleotides, at least 1825 nucleotides, at least 1850 nucleotides, at least 1875 nucleotides, at least 1900 nucleotides, at least 1925 nucleotides, at least 1950 nucleotides, at least 1975 nucleotides, at least 2000 nucleotides, at least 2025 nucleotides, at least 2050 nucleotides, at least 2075 nucleotides, at least 2100 nucleotides, at least 2125 nucleotides, at least 2150 nucleotides, at least 2175 nucleotides, at least 2200 nucleotides, at least 2225 nucleotides, at least 2250 nucleotides, at least 2275 nucleotides, at least 2300 nucleotides, at least 2325 nucleotides, at least 2350 nucleotides, at least 2375 nucleotides, at least 2400 nucleotides, at least 2425 nucleotides, at least 2450 nucleotides, at least 2475 nucleotides, at least 2500 nucleotides, at least 2525 nucleotides, at least 2550 nucleotides, at least 2575 nucleotides, at least 2600 nucleotides, at least 2625 nucleotides, at least 2650 nucleotides, at least 2675 nucleotides, at least 2700 nucleotides, at least 2725 nucleotides, at least 2750 nucleotides, at least 2775 nucleotides, at least 2800 nucleotides, at least 2825 nucleotides, at least 2850 nucleotides, at least 2875 nucleotides, at least 2900 nucleotides, at least 2925 nucleotides, at least 2950 nucleotides, at least 2975 nucleotides or at least 3000 nucleotides.

In some embodiments, the circular nucleic acid molecule is no more than 500 bp, 505 bp, 510 bp, 515 bp, 520 bp, 525 bp, 530 bp, 535 bp, 540 bp, 545 bp, 550 bp, 555 bp, 560 bp, 565 bp, 570 bp, 575 bp, 580 bp, 585 bp, 590 bp, 595 bp, 600 bp, 605 bp, 610 bp, 615 bp, 620 bp, 625 bp, 630 bp, 635 bp, 640 bp, 645 bp, 650 bp, 655 bp, 660 bp, 665 bp, 670 bp, 675 bp, 680 bp, 685 bp, 690 bp, 695 bp, 700 bp, 705 bp, 710 bp, 715 bp, 720 bp, 725 bp, 730 bp, 735 bp, 740 bp, 745 bp, 750 bp, 755 bp, 760 bp, 765 bp, 770 bp, 775 bp, 780 bp, 785 bp, 790 bp, 795 bp, 800 bp, 805 bp, 810 bp, 815 bp, 820 bp, 825 bp, 830 bp, 835 bp, 840 bp, 845 bp, 850 bp, 855 bp, 860 bp, 865 bp, 870 bp, 875 bp, 880 bp, 885 bp, 890 bp, 895 bp, 900 bp, 905 bp, 910 bp, 915 bp, 920 bp, 925 bp, 930 bp, 935 bp, 940 bp, 945 bp, 950 bp, 955 bp, 960 bp, 965 bp, 970 bp, 975 bp, 980 bp, 985 bp, 990 bp, 995 bp, 1000 bp, 1025 bp, 1050 bp, 1075 bp, 1100 bp, 1125 bp, 1150 bp, 1175 bp, 1200 bp, 1225 bp, 1250 bp, 1275 bp, 1300 bp, 1325 bp, 1350 bp, 1375 bp, 1400 bp, 1425 bp, 1450 bp, 1475 bp, 1500 bp, 1525 bp, 1550 bp, 1575 bp, 1600 bp, 1625 bp, 1650 bp, 1675 bp, 1700 bp, 1725 bp, 1750 bp, 1775 bp, 1800 bp, 1825 bp, 1850 bp, 1875 bp, 1900 bp, 1925 bp, 1950 bp, 1975 bp, 2000 bp, 2025 bp, 2050 bp, 2075 bp, 2100 bp, 2125 bp, 2150 bp, 2175 bp, 2200 bp, 2225 bp, 2250 bp, 2275 bp, 2300 bp, 2325 bp, 2350 bp, 2375 bp, 2400 bp, 2425 bp, 2450 bp, 2475 bp, 2500 bp, 2525 bp, 2550 bp, 2575 bp, 2600 bp, 2625 bp, 2650 bp, 2675 bp, 2700 bp, 2725 bp, 2750 bp, 2775 bp, 2800 bp, 2825 bp, 2850 bp, 2875 bp, 2900 bp, 2925 bp, 2950 bp, 2975 bp, 3000 bp, 3025 bp, 3050 bp, 3075 bp, 3100 bp, 3125 bp, 3150 bp, 3175 bp, 3200 bp, 3225 bp, 3250 bp, 3275 bp, 3300 bp, 3325 bp, 3350 bp, 3375 bp, 3400 bp, 3425 bp, 3450 bp, 3475 bp, 3500 bp, 3525 bp, 3550 bp, 3575 bp, 3600 bp, 3625 bp, 3650 bp, 3675 bp, 3700 bp, 3725 bp, 3750 bp, 3775 bp, 3800 bp, 3825 bp, 3850 bp, 3875 bp, 3900 bp, 3925 bp, 3950 bp, 3975 bp, 4000 bp, 4025 bp, 4050 bp, 4075 bp, 4100 bp, 4125 bp, 4150 bp, 4175 bp, 4200 bp, 4225 bp, 4250 bp, 4275 bp, 4300 bp, 4325 bp, 4350 bp, 4375 bp, 4400 bp, 4425 bp, 4450 bp, 4475 bp, 4500 bp, 4525 bp, 4550 bp, 4575 bp, 4600 bp, 4625 bp, 4650 bp, 4675 bp, 4700 bp, 4725 bp, 4750 bp, 4775 bp, 4800 bp, 4825 bp, 4850 bp, 4875 bp, 4900 bp, 4925 bp, 4950 bp, 4975 bp, 5000 bp, 5025 bp, 5050 bp, 5075 bp, 5100 bp, 5125 bp, 5150 bp, 5175 bp, 5200 bp, 5225 bp, 5250 bp, 5275 bp, 5300 bp, 5325 bp, 5350 bp, 5375 bp, 5400 bp, 5425 bp, 5450 bp, 5475 bp, 5500 bp, 5525 bp, 5550 bp, 5575 bp, 5600 bp, 5625 bp, 5650 bp, 5675 bp, 5700 bp, 5725 bp, 5750 bp, 5775 bp, 5800 bp, 5825 bp, 5850 bp, 5875 bp, 5900 bp, 5925 bp, 5950 bp, 5975 bp, 6000 bp, 6025 bp, 6050 bp, 6075 bp, 6100 bp, 6125 bp, 6150 bp, 6175 bp, 6200 bp, 6225 bp, 6250 bp, 6275 bp, 6300 bp, 6325 bp, 6350 bp, 6375 bp, 6400 bp, 6425 bp, 6450 bp, 6475 bp, 6500 bp, 6525 bp, 6550 bp, 6575 bp, 6600 bp, 6625 bp, 6650 bp, 6675 bp, 6700 bp, 6725 bp, 6750 bp, 6775 bp, 6800 bp, 6825 bp, 6850 bp, 6875 bp, 6900 bp, 6925 bp, 6950 bp, 6975 bp, 7000 bp, 7025 bp, 7050 bp, 7075 bp, 7100 bp, 7125 bp, 7150 bp, 7175 bp, 7200 bp, 7225 bp, 7250 bp, 7275 bp, 7300 bp, 7325 bp, 7350 bp, 7375 bp, 7400 bp, 7425 bp, 7450 bp, 7475 bp, 7500 bp, 7525 bp, 7550 bp, 7575 bp, 7600 bp, 7625 bp, 7650 bp, 7675 bp, 7700 bp, 7725 bp, 7750 bp, 7775 bp, 7800 bp, 7825 bp, 7850 bp, 7875 bp, 7900 bp, 7925 bp, 7950 bp, 7975 bp, 8000 bp, 8025 bp, 8050 bp, 8075 bp, 8100 bp, 8125 bp, 8150 bp, 8175 bp, 8200 bp, 8225 bp, 8250 bp, 8275 bp, 8300 bp, 8325 bp, 8350 bp, 8375 bp, 8400 bp, 8425 bp, 8450 bp, 8475 bp, 8500 bp, 8525 bp, 8550 bp, 8575 bp, 8600 bp, 8625 bp, 8650 bp, 8675 bp, 8700 bp, 8725 bp, 8750 bp, 8775 bp, 8800 bp, 8825 bp, 8850 bp, 8875 bp, 8900 bp, 8925 bp, 8950 bp, 8975 bp, 9000 bp, 9025 bp, 9050 bp, 9075 bp, 9100 bp, 9125 bp, 9150 bp, 9175 bp, 9200 bp, 9225 bp, 9250 bp, 9275 bp, 9300 bp, 9325 bp, 9350 bp, 9375 bp, 9400 bp, 9425 bp, 9450 bp, 9475 bp, 9500 bp, 9525 bp, 9550 bp, 9575 bp, 9600 bp, 9625 bp, 9650 bp, 9675 bp, 9700 bp, 9725 bp, 9750 bp, 9775 bp, 9800 bp, 9825 bp, 9850 bp, 9875 bp, 9900 bp, 9925 bp, 9950 bp, 9975 bp, or 10000 bp in length.

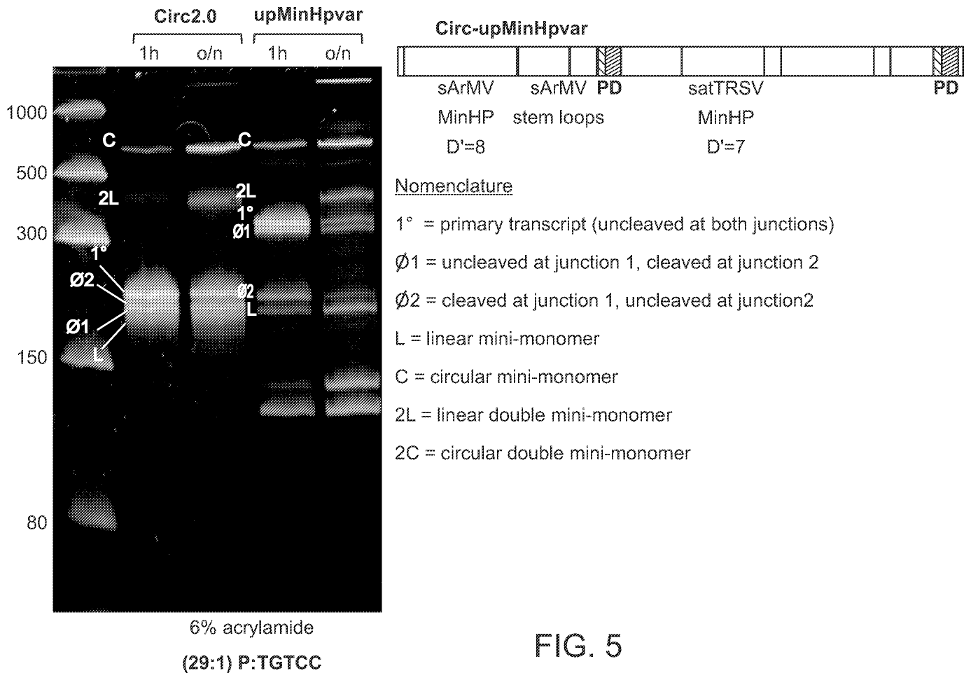

In some embodiments, an in-vitro transcription (IVT) reaction allows determination of the ability of the upstream and/or downstream ribozymes to cleave at the P, D, and/or PD junction as well as the ability of the central catalytic core to undergo circularization (see FIGS. 5-7 and 9-12). At low concentrations of the IVT template, the following products may be present: unprocessed RNA, linear RNA lacking only the P region upstream of the central catalytic core, linear RNA lacking only the D region downstream of the central catalytic core, linear RNA lacking both the P region upstream and the D region downstream of the catalytic core, and circular RNA. At higher concentrations of the IVT template, in addition to the above-listed products multimers of linearized product and circularized product can be formed.

In some embodiments, the nucleic acid molecule comprises a sequence that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to any one of the circular RNA or plasmid sequences described herein.

### Isolating Circularized Nucleic Acid Molecules

In certain embodiments, the step of isolating the circularized molecules can be performed using any appropriate methodology known in the art. Examples of such methodologies are described in, e.g., Green and Sambrook, Molecular Cloning, A Laboratory Manual, 4th Ed., Cold Spring Harbor Press, (2012). For example, provided herein are methods of purifying circular molecules, comprising running the polynucleotide through a size-exclusion column in tris-EDTA or citrate buffer in a high performance liquid chromatography (HPLC) system. In another embodiment, the polynucleotide is run through the size-exclusion column in tris-EDTA or citrate buffer at pH in the range of about 4-7 at a flow rate of about 0.01-5 mL/minute. In one embodiment, the HPLC removes one or more of: intron fragments, nicked linear RNA, linear and circular concatenations, and impurities resulting from the in vitro transcription and splicing reactions.

In certain aspects, provided herein are methods of making circular RNA, said method comprising using a nucleic acid molecule provided herein. In some embodiments, the method comprises a.) synthesizing RNA by in vitro transcription of a nucleic acid molecule, and b.) incubating the RNA in the presence of magnesium ions and quanosine nucleotide or nucleoside at a temperature at which RNA circularization occurs (e.g., between 20° C. and 60° C.).

### Plasmids and Viral Replicating Vectors

In some embodiments, provided herein are DNA plasmids and viral replicating vectors comprising DNA nucleic acid molecules as described above and herein. In some embodiments, the entire size of the DNA plasmids designed are from about 2000 bp to about 15,000 bp. Generally, the plasmid backbone comprises an origin of replication and an expression cassette for expressing a sequence of interest and/or a selection gene. In some aspects, the expression cassette for expressing a selection gene is in the antisense orientation from the central ribozyme. The selection gene can be any marker known in the art for selection of a host cell that has been transformed with a desired plasmid. In some aspects, the selection marker comprises a polynucleotide encoding a gene or protein conferring antibiotic resistance, heat tolerance, fluorescence, or luminescence.

In some embodiments, viral replicating vectors can be used to express the DNA or RNA constructs as described. In planta, gemini viruses are a representative DNA virus that can be used as an expression system (reviewed in, e.g., Hefferon, Vaccines (2014) 2:642-53). In animal cells, there are more choices. Plasmid expression constructs containing viral origins of replication, while not truly viral replicating systems, are stably maintained in cells. Truly replicating viral systems of use include, without limitation, adenovirus, adeno-associated virus, baculovirus, and Vaccinia virus vectors, which are known in the art.

Transcribing a DNA Construct into RNA In Vitro

In some aspects, the one or more DNA constructs, as described above and herein, are first transcribed in vitro into RNA and then the RNA transcript is transfected into a host cell. The step of transcribing the one or more DNA constructs into RNA in vitro can be performed using any methodologies known in the art. In vitro transcription of one or more (e.g., a population of) DNA constructs comprising a library of inserts containing a nucleic acid sequence of interest can be achieved using purified RNA polymerases, e.g. T7 RNA polymerase. Such methodologies are described in, e.g., Green and Sambrook, Molecular Cloning, A Laboratory Manual, 4th Ed., Cold Spring Harbor Press, (2012).

Transfecting a Host Cell with the DNA or RNA Nucleic Acid Molecules Described Herein

In another embodiment, provided herein is a method of expressing protein in a cell, said method comprising transfecting the circular RNA into the cell. In one embodiment, the method comprises transfecting using lipofection or electroporation. In another embodiment, the circular RNA is transfected into a cell using a nanocarrier. In yet another embodiment, the nanocarrier is a lipid, polymer or a lipo-polymeric hybrid.

In some aspects, the DNA construct or in vitro transcribed RNA construct is transfected into a suitable host cell of closed circular DNA plasmid using any method known in the art, e.g., by electroporation of protoplasts, fusion of liposomes to cell membranes, cell transfection methods using calcium ions or PEG, use of gold or tungsten microparticles coated with plasmid with the gene gun. Such methodologies are described in, e.g., Green and Sambrook, Molecular Cloning, A Laboratory Manual, 4th Ed., Cold Spring Harbor Press, (2012). In some embodiments, cells of eukaryotic organisms (plants, animals, fungi, etc.) can be used. In some aspects, the host cell is a prokaryotic cell, e.g., a bacterial cell, an archaeal cell, or an archaebacterial cell.

### In Vivo Transcription

In certain embodiments, for in vivo transcription of a full length nucleic acid molecule, the nucleic acid molecule comprises a binding site is active and induces transcription in the host cell that comprises the nucleic acid molecule. For example, if a DNA construct is introduced into a eukaryotic cell, a selected 5′ or upstream binding site is biologically active for generating RNA in the eukaryotic cell. As appropriate, the 5′ or upstream binding site can be a mammalian promoter that actively promotes transcription in a mammalian host cell. In some aspects, the 5′ or upstream binding site can be a plant binding site that actively promotes transcription in a plant host cell.

### Pharmaceutical Compositions

In embodiments of the present disclosure, the circular nucleic acid molecule products described herein and/or produced using the nucleic acid molecules and/or methods described herein, may be provided in compositions, e.g., pharmaceutical compositions.

In some embodiments, provided herein are compositions, e.g., compositions comprising a circular nucleic acid molecule and a pharmaceutically acceptable carrier. In one aspect, the present disclosure provides pharmaceutical compositions comprising an effective amount of a circular nucleic acid molecule described herein and a pharmaceutically acceptable excipient. Pharmaceutical compositions of the present disclosure may comprise a circular RNA as described herein, in combination with one or more pharmaceutically or physiologically acceptable carriers, excipients or diluents. In some embodiments, pharmaceutical compositions of the present disclosure may comprise a circular nucleic acid molecule expressing cell, e.g., a plurality of circular nucleic acid molecule-expressing cells, as described herein, in combination with one or more pharmaceutically or physiologically acceptable carriers, excipients or diluents.

In some embodiments, a pharmaceutically acceptable carrier can be an ingredient in a pharmaceutical composition, other than an active ingredient, which is nontoxic to the subject. A pharmaceutically acceptable carrier can include, but is not limited to, a buffer, excipient, stabilizer, or preservative. Examples of pharmaceutically acceptable carriers are solvents, dispersion media, coatings, antibacterial and antifungal agents, isotonic and absorption delaying agents, and the like that are physiologically compatible, such as salts, buffers, saccharides, antioxidants, aqueous or non-aqueous carriers, preservatives, wetting agents, surfactants or emulsifying agents, or combinations thereof. The amounts of pharmaceutically acceptable carrier(s) in the pharmaceutical compositions may be determined experimentally based on the activities of the carrier(s) and the desired characteristics of the formulation, such as stability and/or minimal oxidation.

In some embodiments, such compositions may comprise buffers such as acetic acid, citric acid, histidine, boric acid, formic acid, succinic acid, phosphoric acid, carbonic acid, malic acid, aspartic acid, Tris buffers, HEPPSO, HEPES, neutral buffered saline, phosphate buffered saline and the like; carbohydrates such as glucose, sucrose, mannose, or dextrans, mannitol; proteins; polypeptides or amino acids such as glycine; antioxidants; chelating agents such as EDTA or glutathione; adjuvants (e.g., aluminum hydroxide); antibacterial and antifungal agents; and preservatives.

In certain embodiments, compositions of the present disclosure can be formulated for a variety of means of parenteral or non-parenteral administration. In one embodiment, the compositions can be formulated for infusion or intravenous administration. Compositions disclosed herein can be provided, for example, as sterile liquid preparations, e.g., isotonic aqueous solutions, emulsions, suspensions, dispersions, or viscous compositions, which may be buffered to a desirable pH.

### EXAMPLES

The following examples are offered to illustrate, but not to limit.

### Example 1—In Vitro Transcription (IVT)

In vitro transcription reactions were performed at 37° C. for 1 hour or overnight, with the following standard reaction composition: 1× RNA Polymerase Reaction Buffer, 0.5 mM rNTPs (each), 5 mM DTT, 5 U/μL T7 RNA Polymerase (NEB, Ipswich, MA), 1 U/μL RNaseOUT Recombinant Ribonuclease Inhibitor (Invitrogen, Carlsbad, CA), and 10 ng/μL DNA template. RNA transcripts were purified using the RNeasy Mini Kit (QIAGEN, Germantown, MD), as directed by the manufacturer. Except where otherwise stated, RNA samples were visualized by size separation on Novex precast 6% acrylamide, TBE-Urea gels (Invitrogen, Carlsbad, CA), heated with a circulating waterbath to 50 degrees C., then stained with 1× SYBR Gold Nucleic Acid Gel Stain (Invitrogen, Carlsbad, CA) in 1× TBE for 10-15 min. Approximately 250 ng of RNA sample was loaded per well, as determined by quantification on a NanoDrop Spectrophotometer (ThermoFisher Scientific, Carlsbad CA). Electrophoretic densitometry of RNA bands was performed by measuring the area of individual RNA peaks using the Analyze>Gels function in FIJI (Ferreira and Rasband, 2012).

### Example 2—IVT Template and Construct Generation

DNA templates used for in vitro transcription were generated by PCR amplification. The template for Circ2.0 was amplified from plasmid pCirc2.0-PTGTCC (SEQ ID NO:8) using primers Xho T7 Left upper (5′-CTCTCTCGAGTAATACGACTCACTATAGGGTGTCCGTCGAGTCTCCGTTGGA-3′; SEQ ID NO:9) and PTGTCC 2nd PD L (5′-ACGGAGACTCGACGGACAGTGGCTGACAGTTTCCTGTCAGCCACGGCACACCCC TG-3′; SEQ ID NO:10).

Upstream Homo sapiens hammerhead ribozyme variants of the mini-monomer construct were assembled by digesting plasmid pCirc2.0-PTGTCC (SEQ ID NO:8) and gBlock DNA fragment HhsHH up TGTCC (5′-AGTGCGAGCTCGAGCCGTTACCTCGACCTGATGAGCTCCAAAAAGAGCGAAACC TATTAGGTCGTCGAGTACTGGGTTGGAATTCTCGGGTGCCAAGGATAGTACTCAG AAGACAACCAGAGAAACACACGTTGTGGTATATTACCTGGTGGCGCGCCTGAGG TT-3′; SEQ ID NO:11) with AscI (New England BioLabs, Ipswich, MA), combining the AscI-digested fragments together and ligating with T4 DNA ligase (New England BioLabs, Ipswich, MA), and then mixing the ligation mixture with ProNex Chemistry (Promega, Madison, WI) magnetic resin at 1:1 ratio and performing DNA cleanup as directed by the manufacturer for size-selective purification of DNA fragments larger than 1 kb.

Circ-upHsHH-PTGTCC (SEQ ID NO: 12) was amplified using the primers T7 up Hs HH (5′-TAATACGACTCACTATAGGAGTGCGAGCTCGAGCCGT-3′; SEQ ID NO:13) and PTGTCC 2nd PD for upHH L (5′-AGTACTCGACGGACAGTGGCTGACAGTTTCCTGTCAGCCACGGCACACCCCTG-3′; SEQ ID NO:14), and Circ-upHsHH-D′7/4 (SEQ ID NO:15) with primers T7 up Hs HH (SEQ ID NO:13) and PTGTCC 2nd PD L (SEQ ID NO:14).

Circ-upHsHH-D′4/7 (SEQ ID NO:16) was generated essentially as described for Circ-upHsHH-D′7/4 (SEQ ID NO:15), except that the PCR template was assembled by digesting pCirc2.0-PTGTCC (SEQ ID NO:8) and HhsHH up TGTCC (SEQ ID NO:11) with EcoRI-HF (New England BioLabs, Ipswich, MA) instead of AscI.

Template for the in vitro transcription of mini-monomer appended with an upstream satellite arabis mosaic virus (sArMV) E48 ribozyme was generated by digesting plasmid pCirc2.0-PTGTCC (SEQ ID NO:8) and gBlock DNA fragment E48var up TGTCC (5′-AGTGCGAGCTCGAGGAGACTCAGAAGACAAACGGCGAAACACACCTTGTGTGGT ATATTACCCGTTGGAGATTCCAGAGGATTGGTTACCTATCTCCCATGCCCATGTC GGCATTGTCCGTCGAGTCTCCGTTGGAATTCTCGGGTGCCAAGGATGAGACTCAG AAGACAACCAGAGAAACACAC-3′; SEQ ID NO:17) with EcoRI-HF, combining the EcoRI-digested fragments together and ligating with T4 DNA, cleaning up the ligation reaction with ProNex Chemistry (Promega, Madison, WI) at 1:1 ratio, and amplifying the desired ligation products by PCR using primers T7 up E48 upper (5′-TAATACGACTCACTATAGGAGTGCGAGCTCGAGGAGA-3′; SEQ ID NO: 18) and PTGTCC 2nd PD L (SEQ ID NO:10) to generate construct Circ-upE48var (SEQ ID NO:19).

Template for the in vitro transcription of Circ-upSmHH-D′11/5 (SEQ ID NO:20), which possesses an upstream Schistosoma mansoni hammerhead (SmHH) ribozyme, was generated by digesting plasmid pCirc2.0-PTGTCC (SEQ ID NO:8) and gBlock DNA fragment SmHH up TGTCC (5′-AGTGCGAGCTCGAGGGATGTACCTCGACCTGATGAGTCCCAAATAGGACGAAAC GCCGAAAGGCGTCGTCGAGTCCAATCCGTTGGAATTCTCGGGTGCCAAGGATTG GACTCAGAAGACAACCAGAGAAACACACGTTGTGGTATATTACCTGGTGGCGCG CCTGAGGTT-3′; SEQ ID NO:21) with AscI, combining the AscI-digested fragments together and ligating with T4 DNA, cleaning up the ligation reaction with ProNex Chemistry (Promega, Madison, WI) at 1:1 ratio, and amplifying the desired ligation products by PCR using primers T7 up Sm HH (5′-TAATACGACTCACTATAGGAGTGCGAGCTCGAGGGAT-3′; SEQ ID NO:22) and PTGTCC 2nd PD L (SEQ ID NO:10).

Template for the in vitro transcription of Circ-upSmHH-D′5+4/7 (SEQ ID NO:23) was generated essentially as described for Circ-upSmHH-D′ 11/5 (SEQ ID NO:20), except that pCirc2.0-PTGTCC (SEQ ID NO:8) and gBlock SmHH up TGTCC were digested with EcoRI-HF instead of AscI.

To generate plasmid pCirc3.1-HDV (SEQ ID NO:24), which harbors a promoterless version of Circ-upHsHH (P=TGTCC; SEQ ID NO:25) and downstream hepatitis delta virus (HDV) ribozyme, the relevant sequences were PCR amplified using primers BglII HsHH-up (5′-TATATAGATCTCCGTTACCTCGACCTGATGAG-3′; SEQ ID NO:26) and V1aRlower (5′-CGTCTAGATGGCTCTCCCTTAGCCATCCGAGTGGACGTG-3′; SEQ ID NO:27), with the same ligation product used for PCR amplification of Circ-upHsHH-PTGTCC (SEQ ID NO:25) serving as template. The resulting PCR product was gel purified from a 1.8% agarose 1× TAE gel, and G-tailed by incubation with 0.3 U Klenow Fragment (3′-->5′ exo-) and 0.1 mM dGTP in 1× NEBuffer 2 (NEB, Ipswich, MA) at 37° C. for 30 minutes. The G-tailed fragment was then ligated at room temperature to cloning vector DtoR Blue 3 (SEQ ID NO:28) digested with AhdI to produce compatible C overhangs on either end of the linearized plasmid. NEB5-alpha competent cells (NEB, Ipswich, MA) were transformed with the ligation reaction, as directed by the manufacturer, and transformants screened on solid LB media containing 100 μg/mL carbenicillin.

Variant P construct, pCirc-T7HsHH-PCGGTA (SEQ ID NO:29), was assembled by PCR amplification of plasmid pCirc3.0-PCGGTA (SEQ ID NO:30) with primers Fse DtoRO lower (5′-ATCGGCCGGCCCGCGGAACCCCTATTTGTTTATTTTTCTAAATAC-3′; SEQ ID NO:31) and Eco E48core PCGGTC DHsHH Upper (5′-GGGTTGGAATTCTCGGGTGCCAAGGATAGTACTCAGAAACCGAC-3′; SEQ ID NO:32) to modify the D′ sequence of satTRSV catalytic core to match the D sequence of HsHH followed by digesting both the purified PCR amplicon and gBlock HhsHH up TGTCC (SEQ ID NO:11) with EcoRI-HF, combining the EcoRI-digested fragments together and ligating with T4 DNA, cleaning up the ligation reaction with ProNex Chemistry (Promega, Madison, WI) at 1:1 ratio, and amplifying the desired ligation products by PCR using primers T7 up Hs HH (SEQ ID NO:13) and DtoRseq2 (5′-CACCTCTGACTTGAGCGTCGATTT-3′; SEQ ID NO: 33). The resulting PCR product was gel purified, G-tailed, and cloned into AhdI-digested DtoR Blue 3 (SEQ ID NO:28), as outlined above for pCirc3.1-HDV (SEQ ID NO:24).

Plasmid pCirc3.1-HDV (SEQ ID NO:24) served as the template for PCR amplification of the DNA fragments used for in vitro transcription of Circ-upHsHH (SEQ ID NO:25) with differing lengths of the stem loop structure positioned between the Insulator′ and downstream P sequence. Circ-upHsHH-5bpstem (SEQ ID NO:34) was amplified with primers T7upHsHHextend (5′-TAATACGACTCACTATAGGAGATCTCCGTTACCTCGACCTGATGAG-3′; SEQ ID NO:35) and V1a 5 nt 2nd PD L (5′-CCAGTACTCGACGGACAGTGGCTTTCGCCACGGCACACC-3′; SEQ ID NO:36), Circ-upHsHH-7bpstem (SEQ ID NO:37) with primers T7upHsHHextend (SEQ ID NO:35) and V1a 7 nt 2nd PD L (5′-CCAGTACTCGACGGACAGTGGCTGTTTCCAGCCACGGCACACCCCTG-3′; SEQ ID NO:38), Circ-upHsHH-9bpstem (SEQ ID NO:39) with primers T7upHsHHextend (SEQ ID NO:35) and V1a 9 nt 2nd PD L (5′-CCAGTACTCGACGGACAGTGGCTGACTTTCGTCAGCCACGGCACACCCCTG-3′; SEQ ID NO:40), and Circ-upHsHH-11bpstem (SEQ ID NO:41) with primers T7upHsHHextend (SEQ ID NO:35) and V1a 11 nt 2nd PD L (5′-CCAGTACTCGACGGACAGTGGCTGACAGTTTCCTGTCAGCCACGGCACACCCCTG-3′; SEQ ID NO:42).

To determine if the addition of a downstream sArMV E48 ribozyme enhances RNA processing and/or circularization of Circ-upHsHH-PTGTCC (SEQ ID NO:25), plasmid pCirc3.1-HDV (SEQ ID NO:24) and gBlock DNA fragment E48var down TGTCC (5′-AGTGCGAGCCTGCAGGGGTGTGCCGTGGCTGACAGGAAACTGTCAGCCACTGTC CGTCGAGTCTCCGTATGAGACTCAGAAGACAAACGGCGAAACACACCTTGTGTG GTATATTACCCGTTTCTAGACTGAGGTT-3′; SEQ ID NO:43) with SbfI (New England BioLabs, Ipswich, MA), combining the SbfI-digested fragments together and ligating with T4 DNA, cleaning up the ligation reaction with ProNex Chemistry (Promega, Madison, WI) at 1:1 ratio, and amplifying the desired ligation products by PCR using primers T7upHsHHextend (SEQ ID NO:35) and Downstream E48 PTGTCC (5′-AACCTCAGTCTAGAAACGGGTAA-3′; SEQ ID NO:44) to generate the in vitro transcription template Circ-upHsHH/dnE48var (SEQ ID NO:45).

To determine if the addition of a downstream SmHH ribozyme enhances RNA processing and/or circularization of Circ-upHsHH-PCGGTC (SEQ ID NO:12), plasmid pCirc-T7HsHH-PCGGTA (SEQ ID NO:29) and gBlock DNA fragment SmHH down CGGTC (5′-AGTGCGAGCCTGCAGGGGTGTGCCGTGGCTGACAGGAAACTGTCAGCCACCGGT CCTGGTATCCAATCCGAAAGGATGTACCTCGACCTGATGAGTCCCAAATAGGAC GAAACCGGTCTAGACTGAGGTT-3′; SEQ ID NO:46) with SbfI (New England BioLabs, Ipswich, MA), combining the SbfI-digested fragments together and ligating with T4 DNA, cleaning up the ligation reaction with ProNex Chemistry (Promega, Madison, WI) at 1:1 ratio, and amplifying the desired ligation products by PCR using primers T7 up Hs HH (SEQ ID NO:13) and Downstream PLMV Sm HH PCGGTC (5′-AACCTCAGTCTAGACCGGTTTC-3′; SEQ ID NO:47) to generate the in vitro transcription template Circ-upHsHH/dnSmHH (SEQ ID NO:48). The template for in vitro transcription of Circ-upHsHH-PCGGTC (SEQ ID NO:12) was amplified from plasmid pCirc-T7HsHH-PCGGTA (SEQ ID NO:29) using primers T7 up Hs HH (SEQ ID NO:13) and PCGGTC 2nd PfixedD L (5′-AGTACTCGACGACCGGTGGCTGACAGTTTCCTGTCAGCCACGGCACACCCCTG-3′; SEQ ID NO:49).

Template for in vitro transcription of Circ-dnPLMVHH (SEQ ID NO:50), which harbors a downstream peach latent mosaic viroid hammerhead (PLMV HH) ribozyme, was obtained by digesting gBlock DNA fragment PLMVHH down CGGTC (5′-AGTGCGAGCCTGCAGGGGTGTGCCGTGGCTGACAGGAAACTGTCAGCCACCGGT CTGTGCTAAGCACACTGATGAGTCTCTGAAATGAGACGAAACCGGTCTAGACTG AGGTT-3′; SEQ ID NO:51) with SbfI and ligating to SbfI-digested plasmid pCirc3.0-PCGGTA, (SEQ ID NO:30) DNA purification using ProNex Chemistry at 1:1 ratio, and PCR amplification of the desired ligation product with primers T7 left upper PCGGTC (5′-TAATACGACTCACTATAGGGCTCGAGGCTAGCCGGTCGTCGAGTC-3; SEQ ID NO:52) and Downstream PLMV Sm HH PCGGTC (SEQ ID NO:47).

Template for in vitro transcription of Circ-upHsHH/dnPLMVHH (SEQ ID NO:53) was generated by digesting gBlock DNA fragment PLMVHH down CGGTC (SEQ ID NO:51) with SbfI and ligating to SbfI-digested plasmid pCirc-T7HsHH-PCGGTA (SEQ ID NO:29), DNA purification using ProNex Chemistry at 1:1 ratio, and PCR amplification of the desired ligation product with primers T7 up Hs HH (SEQ ID NO:13) and Downstream PLMV Sm HH PCGGTC (SEQ ID NO:47).

Construct pCirc3.2 (SEQ ID NO:54) was generated by PCR amplification of fragment Circ-upHsHH-D′7/6 (SEQ ID NO:59) with primers BglII HsHH-up (SEQ ID NO:26) and TGTCC D6/7 2nd PD L Xba (5′-TATATTCTAGACGTACTCGACGGACAGTGGCTGACAGTTTCCTGTCAGCCACGGC ACACCCCTG-3′; SEQ ID NO:55), using plasmid pCirc3.1-HDV (SEQ ID NO:24) as template, and G/C cloning into vector DtoR Blue 3 (SEQ ID NO:28), as described above for pCirc3.1-HDV (SEQ ID NO:24).

Construct pCirc3.1-MinHp-sArMV (SEQ ID NO:57) was generated by G/C cloning of a PCR product amplified using primers BglII HsHH-up (SEQ ID NO:26) and Downstream E48 PTGTCC (SEQ ID NO:44), using the same template DNA used to amplify Circ-upHsHH/dnE48var (SEQ ID NO:45).

Construct pCirc3.1-SmHH (SEQ ID NO:56) was generated by G/C cloning of a PCR product amplified using primers BglII HsHH-up (SEQ ID NO:26) and Downstream PLMV Sm HH (SEQ ID NO:47), using the same template DNA used to amplify Circ-upHsHH/dnSmHH (SEQ ID NO:48).

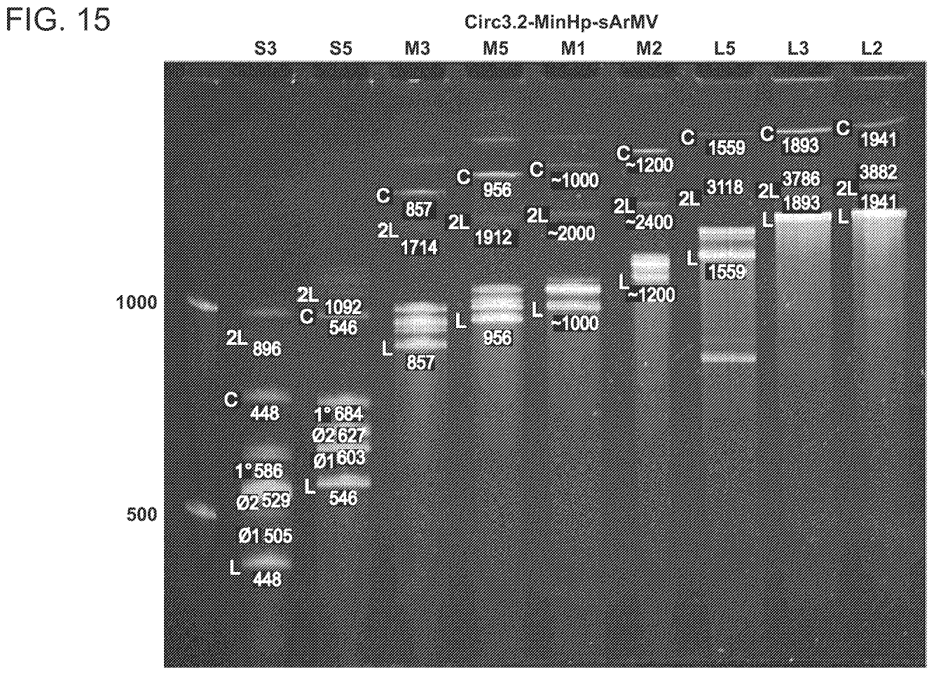

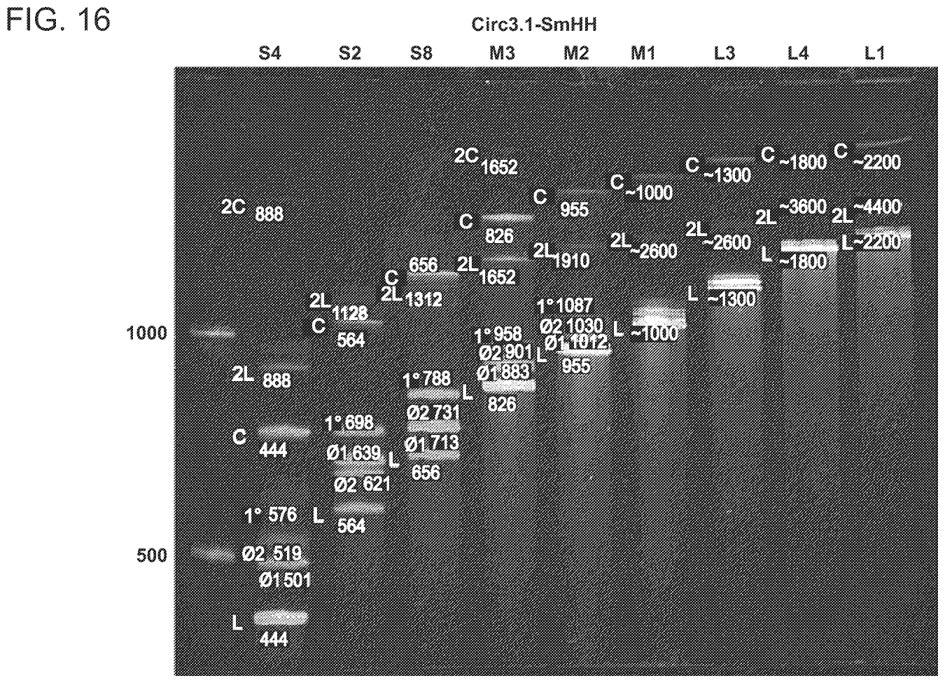

Cloning of random fragments of human male genomic DNA within pCirc3.2 (SEQ ID NO:54), pCirc3.1-HDV (SEQ ID NO:24), pCirc3.1-MinHp-sArMV (SEQ ID NO:57), and pCirc3.1-SmHH (SEQ ID NO:56) was performed by digesting each plasmid with SalI-HF (New England BioLabs), and partially filling in the resulting overhangs with dCTP and dTTP. Human male genomic DNA was partially digested with Sau3AI (New England BioLabs), then partially filled in with dGTP and dATP. The human genomic DNA fragments were then run on a 0.7% agarose 1× TAE gel, and DNA of the approximate size ranges of 0.2-0.5 kb, 0.8-1.2 kb, and 1.5-2.0 kb were excised from the gel and purified using a commercial gel extraction kit. The purified DNA fragments from each of the three size ranges was then ligated to the compatible overhangs of the partially filled-in SalI-digested pCirc3.2 (SEQ ID NO:54), pCirc3.1-HDV (SEQ ID NO:24), pCirc3.1-MinHp-sArMV (SEQ ID NO:57), and pCirc3.1-SmHH (SEQ ID NO:56) plasmid DNA with T4 DNA ligase. The ligation reactions were introduced into NEB5-alpha by heat shock transformation, and selection of transformants performed on solid LB media containing 100 μg/mL carbenicillin. Random colonies were selected to obtain a range of sizes of random genomic DNA inserted between the Insulator and Insulator′ sequences of pCirc3.2 (SEQ ID NO:54), pCirc3.1-HDV (SEQ ID NO:24), pCirc3.1-MinHp-sArMV (SEQ ID NO:57), and pCirc3.1-SmHH (SEQ ID NO:56). Plasmid DNA was isolated and used to generate DNA templates for in vitro transcription by PCR amplification. The primer pair T7upHsHHextend (SEQ ID NO:35) and TGTCC D6/7 2nd PD L (5′-CGTACTCGACGGACAGTGGCTGACAGTTTCCTGTCAGCCACGGCACACCCCTG-3′; SEQ ID NO:58) was used for PCR amplification of derivatives of pCirc3.2 (SEQ ID NO:54), primers T7upHsHHextend (SEQ ID NO:35) and V1aRlower (SEQ ID NO:27) for derivatives of pCirc3.1-HDV (SEQ ID NO:24), primers T7upHsHHextend (SEQ ID NO:35) and Downstream E48 PTGTCC (SEQ ID NO:44) for derivatives of pCirc3.1-MinHp-sArMV (SEQ ID NO:57), and primers T7upHsHHextend (SEQ ID NO:35) and Downstream PLMV Sm HH PCGGTC (SEQ ID NO:47) for derivatives of pCirc3.1-SmHH (SEQ ID NO:56). IVT products were electrophoresed on denaturing acrylamide gels. Results are shown in FIG. 14-16.

XbaI and BglII digested DNA from constructs pCirc1a (SEQ ID NO:75), pCirc3.2 (SEQ ID NO:54), pCirc3.1-HDV (SEQA ID NO:24), pCirc3.1-SmHH (SEQ ID NO:56), and pCirc3.1-MinHp-sArMV (SEQ ID NO:57) were cloned into a PCR product derived from the CMV promoter-containing plasmid pD2610-v6-03 from Atum (formerly DNA2.0) digested with NheI and BamHI. The construct DNAs were ligated to the digested plasmid PCR DNA using 10× T4 DNA ligase buffer and T4 DNA ligase after incubation at 16 C overnight. Ligations were transformed into NEB5alpha chemically competent cells using the manufacturer's protocol and plates on LB kan (50 ug/ul). Colonies with inserts were identified using colony PCR then sequenced. Plasmid preparations were made for each construct and each plasmid preparation received a unique barcode design library cloned into unique AscI and SbfI sites between the two ribozyme cleavage sites. Each barcode library contained approximately 2K to 5K unique barcodes. Endotoxin free plasmids were prepared, then transfected into CHO, HEK293T, and H1299 cells. After one day, RNA was extracted from the cells, reverse transcribed and PCRed, followed by next generation sequencing. Once the sequence data was available, the number of reads were distributed across the five samples using the unique barcode design. After normalization of RNA reads for each design to the plasmid DNA reads for that design, the distribution of each construct's RNA produced relative to each other could be determined. Values were normalized to Circ1a and are presented in Table 2.

Table 2 shows the effect of adding a downstream ribozyme (HDV ribozyme) only, an upstream ribozyme (Homo sapiens hammerhead (HsHH)) only, or adding an upstream ribozyme (Homo sapiens hammerhead (HsHH)) and downstream ribozymes (HDV ribozyme, Schistosoma mansoni hammerhead (SmHH), or sArMV hairpin) on processing and circularization in an in vivo assay performed in HEK273T. Column 1 is the name for the various constructs, column 2 is the type of the upstream ribozyme, column 3 is the type of the downstream ribozyme, column 4 identifies in which figure an example of the type of construct can be found, column 5-7 show the RNA reads/total DNA reads ratio normalized to Circ1a for CHO, HEK293T, and H1299 cells respectively.

TABLE 2

| | | | | CHO | HEK | H1299 |
|---|---|---|---|---|---|---|
| | | | | RNA | RNA | RNA |
| | Upstream | Downstream | | reads/total | reads/total | reads/total |
| | ribozyme | ribozyme | FIG. | DNA reads* | DNA reads* | DNA reads* |
| Circ1a | None | HDV | 3 | 1X | 1X | 1X |
| Circ3.2 | HsHH | None | 4 | 11.2X | 7.0X | 8.5X |
| Circ3.1 with HDV | HsHH | HDV | 8 | 25.0X | 15.5X | 14.1X |
| Circ3.1 with | HsHH | SmHH | 10 | 5.6X | 2.5X | 2.4X |
| SmHH | | | | | | |
| Circ3.1 with | HsHH | MinHp | 10 | 6.2X | 2.2X | 2.5X |
| MinHp-sArMV | | | | | | |

*Normalized to “Circ1a” construct

pCirc1a (SEQ ID NO: 75):

TGTCCGTCGAGTCTCCGTTGGAATTCTCGGGTGCCAAGGATGAGACTCA
GAAGACAACCAGAGAAACACACGTTGTGGTATATTACCTGGTGGCGCGC
CCCGTTCAGAGTTCTACAGTCCGACGATCGCGGCCGCGTCGACGGATCC
AAGCTTCCTGCAGGGGTGTGCCGTGGCGAAAGCCACTGTCCGGGTCGGC
ATGGCATCTCCACCTCCTCGCGGTCCGACCTGGGCATCCGAAGGAGGAC
GCACGTCCACTCGGATGGCTAAGGGAGAGCCA

### Example 3: Generation of Circular RNA Molecules

This Example describes the creation of circular RNA molecules using certain embodiments of the invention, containing a variety of IRES sequences. A ribozyme 1-CVB3 IRES-Gaussia luciferase CDS-ribozyme 2 fragment containing the following sequence was ordered as a gBlock from IDT DNA.

ribozyme 1-CVB3 IRES-Gaussia luciferase CDS-ribozyme 2 fragment (SEQ ID NO: 76):

CTCTCTGGATCCTAATACGACTCACTATAGGAGTGCGAGCTCGAGCCGTT
ACCTCGACCTGATGAGCTCCAAAAAGAGCGAAACCTATTAGGTCGTCGAG
TACTGGGTTGGGAATTCTCGGGTGCCAAGGATAGTACTCAGAATGCGACC
AGAGAAACACACGTTGTGGTATATTACCTGGTACGCCTTGACTAGCTACA
CCGCCGCGCCCACACGAAGGCTACAATCTCCACCTCCAGGTGGTGGCGCG
CCCCTTAAAACAGCCTGTGGGTTGATCCCACCCACAGGCCCATTGGGCGC
TAGCACTCTGGTATCACGGTACCTTTGTGCGCCTGTTTTATACCCCCTCC
CCCAACTGTAACTTAGAAGTAACACACACCGATCAACAGTCAGCGTGGCA
CACCAGCCACGTTTTGATCAAGCACTTCTGTTACCCCGGACTGAGTATCA
ATAGACTGCTCACGCGGTTGAAGGAGAAAGCGTTCGTTATCCGGCCAACT
ACTTCGAAAAACCTAGTAACACCGTGGAAGTTGCAGAGTGTTTCGCTCAG
CACTACCCCAGTGTAGATCAGGTCGATGAGTCACCGCATTCCCCACGGGC
GACCGTGGCGGTGGCTGCGTTGGCGGCCTGCCCATGGGGAAACCCATGGG
ACGCTCTAATACAGACATGGTGCGAAGAGTCTATTGAGCTAGTTGGTAGT
CCTCCGGCCCCTGAATGCGGCTAATCCTAACTGCGGAGCACACACCCTCA
AGCCAGAGGGCAGTGTGTCGTAACGGGCAACTCTGCAGCGGAACCGACTA
CTTTGGGTGTCCGTGTTTCATTTTATTCCTATACTGGCTGCTTATGGTGA
CAATTGAGAGATCGTTACCATATAGCTATTGGATTGGCCATCCGGTGACT
AATAGAGCTATTATATATCCCTTTGTTGGGTTTATACCACTTAGCTTGAA
AGAGGTTAAAACATTACAATTCATTGTTAAGTTGAATACAGCAAAATGGG
AGTCAAAGTTCTGTTTGCCCTGATCTGCATCGCTGTGGCCGAGGCCAAGC
CCACCGAGAACAACGAAGACTTCAACATCGTGGCCGTGGCCAGCAACTTC
GCGACCACGGATCTCGATGCTGACCGCGGGAAGTTGCCCGGCAAGAAGCT
GCCGCTGGAGGTGCTCAAAGAGATGGAAGCCAATGCCCGGAAAGCTGGCT
GCACCAGGGGCTGTCTGATCTGCCTGTCCCACATCAAGTGCACGCCCAAG
ATGAAGAAGTTCATCCCAGGACGCTGCCACACCTACGAAGGCGACAAAGA
GTCCGCACAGGGCGGCATAGGCGAGGCGATCGTCGACATTCCTGAGATTC
CTGGGTTCAAGGACTTGGAGCCCATGGAGCAGTTCATCGCACAGGTCGAT
CTGTGTGTGGACTGCACAACTGGCTGCCTCAAAGGGCTTGCCAACGTGCA
GTGTTCTGACCTGCTCAAGAAGTGGCTGCCGCAACGCTGTGCGACCTTTG
CCAGCAAGATCCAGGGCCAGGTGGACAAGATCAAGGGGGCCGGTGGTGAC
TAACCTGCAGGGGTGTGCCACGAGGTAGCAGACCCCTACGCTACGCAGCA
CGGTTCACTAGTTTACGTGTGGCTGACAGGAAACTGTCAGCCACCGCATG
GGTCGGCATGGCATCTCCACCTCCTCGCGGTCCGACCTGGGCATCCGAAG
GAGGACGCACGTCCACTCGGATGGCTAAGGGAGAGCCATCTAGAAGAGA

This fragment was digested with BamHI and XbaI. A PCR fragment was made from pUC19 resulting in a fragment containing the pUC origin of replication and the beta-lactamase gene. This fragment has BglII and XbaI restriction sites added one to each end.

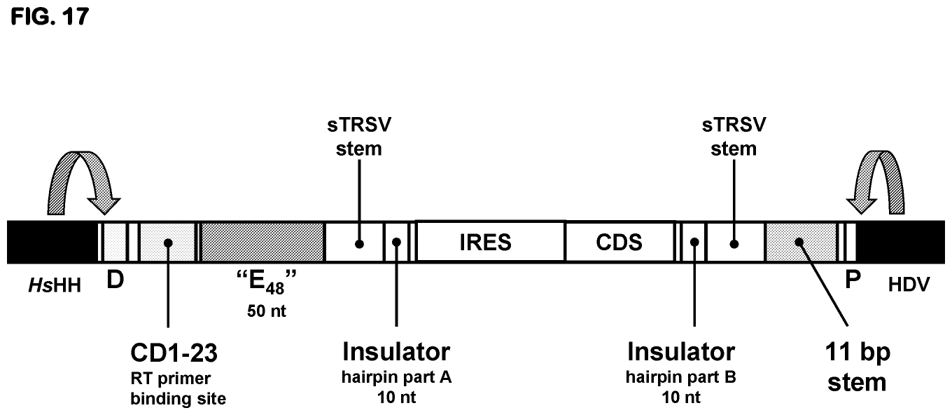

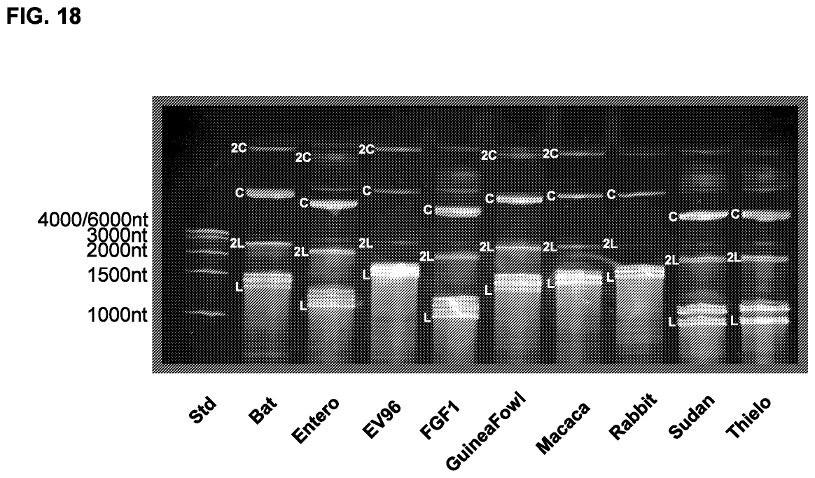

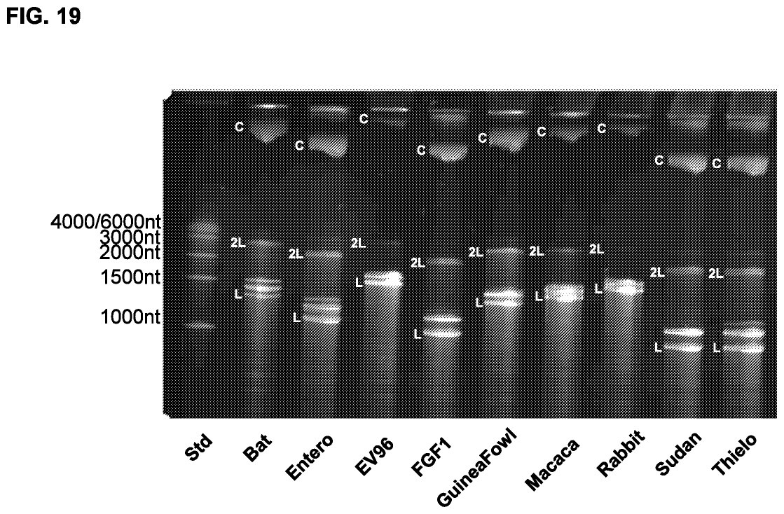

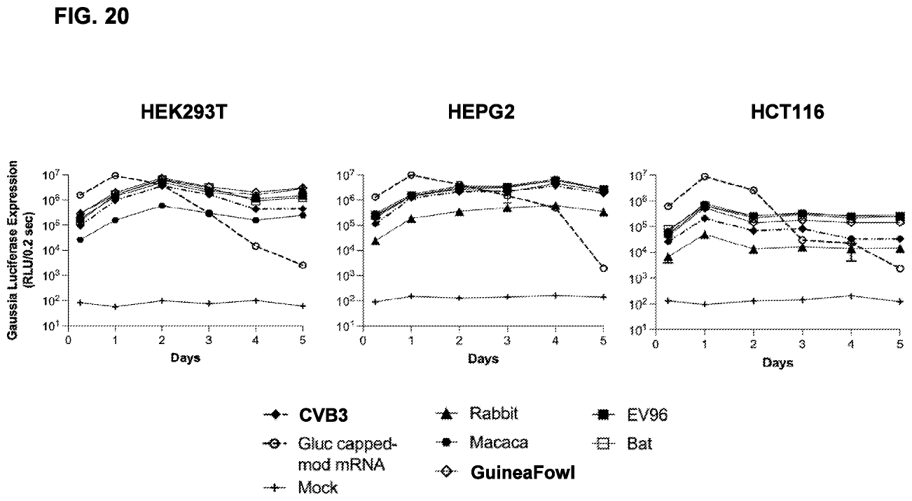

After digest of this fragment with BglII and XbaI, the digested fragment was ligated to the BamHI/XbaI-digested ribozyme 1-CVB3-Gluc CDS-ribozyme 2 fragment with 10× T4 DNA ligase buffer and T4 DNA ligase at 16 C overnight. The ligated DNA was transformed into NEB5alpha chemically competent cells using the manufacturer's protocol then plated on LB carb (100 ug/ul). Colonies were checked by colony PCR. Plasmid from three clones were prepared and the insert was completely sequenced. Plasmid from correct clones was designated as “CVB3”, which, after being used to make in vitro transcripts as described in Example 1, was used as the control in Example 4 and FIG. 20, for the luciferase circular RNA in Example 5 and Table 5, and as the starting material for making the alternative IRES constructs in Table 3, Example 4, and FIGS. 18-20. These CVB3 constructs are represented by the exemplar in FIG. 17.

Constructs were then generated by inserting alternative IRES sequences in place of the CVB3 IRES. Table 3 shows the viral sources of the IRES' used in this Example and in FIG. 18 through FIG. 20.

TABLE 3

| Sample name | Viral source |
|---|---|
| Bat | Bat picornavirus |
| Entero | EV J enterovirus |
| EV96 | EV96 enterovirus |
| FGF1 | Fibroblast growth factor1 human mRNA |
| GuineaFowl | GuineaFowl picornavirus |
| Macaca | Macaca picornavirus |
| Rabbit | Rabbit picornavirus |
| Sudan | Sudan ebolavirus |
| Thielo | Theilers murine encephalomyelitis virus |

Table 4: provides the exemplary IRES sequences used.

### CVB3 (SEQ ID NO:63):

GGATCCTAATACGACTCACTATAGGAGTGCGAGCTCGAGCCGTTACCTCG
ACCTGATGAGCTCCAAAAAGAGCGAAACCTATTAGGTCGTCGAGTACTGG
GTTGGGAATTCTCGGGTGCCAAGGATAGTACTCAGAATGCGACCAGAGAA
ACACACGTTGTGGTATATTACCTGGTACGCCTTGACTAGCTACACCGCCG
CGCCCACACGAAGGCTACAATCTCCACCTCCAGGTGGTGGCGCGCCCCTT
AAAACAGCCTGTGGGTTGATCCCACCCACAGGCCCATTGGGCGCTAGCAC
TCTGGTATCACGGTACCTTTGTGCGCCTGTTTTATACCCCCTCCCCCAAC
TGTAACTTAGAAGTAACACACACCGATCAACAGTCAGCGTGGCACACCAG
CCACGTTTTGATCAAGCACTTCTGTTACCCCGGACTGAGTATCAATAGAC
TGCTCACGCGGTTGAAGGAGAAAGCGTTCGTTATCCGGCCAACTACTTCG
AAAAACCTAGTAACACCGTGGAAGTTGCAGAGTGTTTCGCTCAGCACTAC
CCCAGTGTAGATCAGGTCGATGAGTCACCGCATTCCCCACGGGCGACCGT
GGCGGTGGCTGCGTTGGCGGCCTGCCCATGGGGAAACCCATGGGACGCTC
TAATACAGACATGGTGCGAAGAGTCTATTGAGCTAGTTGGTAGTCCTCCG
GCCCCTGAATGCGGCTAATCCTAACTGCGGAGCACACACCCTCAAGCCAG
AGGGCAGTGTGTCGTAACGGGCAACTCTGCAGCGGAACCGACTACTTTGG
GTGTCCGTGTTTCATTTTATTCCTATACTGGCTGCTTATGGTGACAATTG
AGAGATCGTTACCATATAGCTATTGGATTGGCCATCCGGTGACTAATAGA
GCTATTATATATCCCTTTGTTGGGTTTATACCACTTAGCTTGAAAGAGGT
TAAAACATTACAATTCATTGTTAAGTTGAATACAGCAAAATGGGAGTCAA
AGTTCTGTTTGCCCTGATCTGCATCGCTGTGGCCGAGGCCAAGCCCACCG
AGAACAACGAAGACTTCAACATCGTGGCCGTGGCCAGCAACTTCGCGACC
ACGGATCTCGATGCTGACCGCGGGAAGTTGCCCGGCAAGAAGCTGCCGCT
GGAGGTGCTCAAAGAGATGGAAGCCAATGCCCGGAAAGCTGGCTGCACCA
GGGGCTGTCTGATCTGCCTGTCCCACATCAAGTGCACGCCCAAGATGAAG
AAGTTCATCCCAGGACGCTGCCACACCTACGAAGGCGACAAAGAGTCCGC
ACAGGGCGGCATAGGCGAGGCGATCGTCGACATTCCTGAGATTCCTGGGT
TCAAGGACTTGGAGCCCATGGAGCAGTTCATCGCACAGGTCGATCTGTGT
GTGGACTGCACAACTGGCTGCCTCAAAGGGCTTGCCAACGTGCAGTGTTC
TGACCTGCTCAAGAAGTGGCTGCCGCAACGCTGTGCGACCTTTGCCAGCA
AGATCCAGGGCCAGGTGGACAAGATCAAGGGGGCCGGTGGTGACTAACCT
GCAGGGGTGTGCCACGAGGTAGCAGACCCCTACGCTACGCAGCACGGTTC
ACTAGTTTACGTGTGGCTGACAGGAAACTGTCAGCCACCGCATGGGTCGG
CATGGCATCTCCACCTCCTCGCGGTCCGACCTGGGCATCCGAAGGAGGAC
GCACGTCCACTCGGATGGCTAAGGGAGAGCCATCTAGA

Bat picornavirus SE ID NO:64

CCCCCCCTCCCTCCTTTTCATCGGCTGTCCGTTGCCGTGGAGCTTCACTA
TCCGTAGTGTTGTTCCGGAAAACGGACACAGCAACACCTTCCTCCGGGTT
GGTGCTTGCAATCCCTTTCCCCTTTTGTTGACTTCCCGGACTCATTCGTT
TTGGCTTTCACATTGGCCCTAGAGGCTATCTTGTTGGATTCCTGTACCCT
GGCAATCTGAACTCCTTCGCATGCCCTCCCACATGGTCGAGGACAGTAAG
GCGCGGGACCAAGAGAGCCGGGCACCAGACCCATTTCTGAATTGGACTGC
CACCGCCAAGAATTGCGGAGCGGCTCTGTTGTGTGAGATCCTCCTTTCCC
ACAGATAGCGCATCCCAGTGCGTGTATGGGGGTTAGGGTGCATACAGCAT
CCGCGACTGTGGTCAGATGAGCGGAAGCATCTGCTAGGATAAAGGGGACG
CGATTCGCTATCCCCGAAGCATCCCTCATTGAGGTAAAGCCCTAGTAGGT
GACGGTGGACTGGAATGCCACCCTGGTTCCGTGCAGCTTGACAGAGTGCC
GGCCGGTGCTCTGGCCACAGAAATCGGCTGTTTTGGGACATGC

EV J enterovirus SE ID NO:65

TTACCAATGCTTAATCAGTGAGGCACCTATCTCAGCGATCTGTCTATTTC
GTTCATCCATAGTTGCCTGACTCCCCGTCGTGTAGATAACTACGATACGG
GAGGGCTTACCATCTGGCCCCAGTGCGCGTAAGTCTGGGACGGAACCGAC
TACTTTGGGTGTCCGTGTTTCCTGTTTTACTTACTTTGGCTGCTTATGGT
GACAATCTAGTGTTGTTACCATATAGCTATTGGATTGGCCATCCGGTGTT
TTGAATTGTGTGTTTATACTAATTCTTTTACATATCACAGACAACCAAAT
C

EV96 enterovirus SE ID NO:66

TTAAAACAGCCTGTGGGTTGTTCCCACCCACAGGGCCCACCGGGCGCCAG
CACACTGGTATCACGGTACCCTTGTGCGCCTGTTTTATCCACCCCTTCCC
ACAGTAACTTAGAAGCACACCATGTATACGGTCAATAGGCGGCTCAGTAC
ACCAACTGGGCCACGACCAAGCACTTCTGTTACCCCGGACTGAGTATCAA
TAAGCTGCTCACGCGGCTGAAGGAGAAAACGTTCGTTACCCGGCCAATTA
CTTCGAGAAACCCAGTACCACCATGAAGGTTGCGCAGCGTTTCGTTCCGC
ACAACCCCAGTGTAGATCAGGTCGATGAGTCACCGCGTTCCTCACGGGTG
ACCGTGGCGGTGGCTGCGTTGGCGGCCTGCCCATGGGGCAACCCATGGGA
CGCTTCAATACTGACATGGTGCGAAGAGTCTATTGAGCTAATTGGTAGTC
CTCCGGCCCCTGAATGCGGCTAATCCTAACTGCGGAGCAGATACCCACAA
ACCAGTGGGCAGTCTGTCGTAACGGGCAACTCTGCAGCGGAACCGACTAC
TTTGGGTGTCCGTGTTTCCTTTTATCCTTACACTGGCTGCTTATGGTGAC
AATTGAGAAATTGTTACCATATAGCTATTGGATTGGCCATCCGGTGACAA
ACAGAGCAGTTGTTTACCTATTTGTTGGTTTCGTACCGCTGAACCTTAAA
GTTCTAAAGACCCTCAATTTTATCTTAGCACTTAACACAGCAAAATGC

Fibroblast growth factor1 human mRNA (SEQ ID NO:67)

CTCACCGGCTCCAGATTTATCAGCAATAAACCAGCCAGCCGGAAGGGCCG
AGCGCAGAAGTGGTCCTGCAACTTTATCCGCCCCAGGCAGTGGGGCCGGC
TGGCCAGACTCTTGGGGGATTCCTTAGTGAGTGAGTTCACTGCTCAAAGA
AGGGCTTTGCCACTTCTGCAGGGAAGCCAGCCACGGGCCAGCAGTCTTGA
AAGCGCCACAAGCAGCAGCTGCTGAGGACTTGAAGTCAGCTGAGGGATTT
CCGTAAGCATCGTGATCGATTAGCCTTAAGCCTTGAATCCGAGTCCCATG
C

Guinea Fowl picornavirus (SEQ ID NO:68)

TGCATGTCATTTCTCCCTTCCCCCTCCAATAACCTTTTCCCCCTCTAATC
GGACTGATCAACCGTGTATGATGATTATTGGATTGTGGAGTTATATGATG
GCCTGGCATGGAATCCAGGAATTCTTGCATATTGGAATTGTATCCCACAT
GAACGGGGATGTGGCAAGTCCCCTAGTAGTGCTGGGCTCTACCCAATGTG
TAAGGACCGTGACTTCAAGACTAGTTATGGTCGTGTGACAAAGGGAAGAA
TTTTCTTACCCGCCTGTGTGTTAGGTGCGCACGCTGGGAAACCATAAGCG
ACCAAAGGTAAAGTGGTGCTGAAATATTGCAAGCTCAATGCCTTTGTCGT
GAGTCGGTGCCGCTGTATCTAATACACGCCCCCTTCGGGGTAAATGGCTG
ACACGGGCATCCGCACTTGGGAATGTGCGGCTGGAACTAGACTGGGTGAT
AGCCTGCCGGCTGGCGCTGTTGTCATGGTATAGCCAGTTGATTGCCATAT
GC

Macaca picornavirus (SEQ ID NO:69)

GGAGGATACTTTGTTTAGCTTTGCAATTCTTAAACTGTTTTCCATTTCAC
TGGTCGTTTGACGCTTGTAGGGCGACAGGTGTTCCTAGCTCTTGCTTTTC
TAAACTATCGAATTTTGTTTTCCACTCGTTCATATGTCTATGTATGAATG
AACGGGGTGAGTCCTCGTTGGCCCTCGCTGGAGTGTAAATTCCCAGTCTT
TCTGGAACTAGAATTACACAAGACTCCAGGAGTGTTCTGAAGATTTTCAT
ATTTAAATAAAATCTTTTGGGATTGTCCTTGATGGTTGTAGCGATGTCTA
GTGTGTGTGCGGATTCCCATGCTGGCAACAGCATCCTCACAGGCCAAAAG
CCCAGGGTTAACAGCCCCCGCTAGATGCATGGTACCCCCCATGCCCATTT
TGGATATGAAATTAAGGTTTGTTTACTTAGGAATTTCATAAGGAAGCTTC
TGAGGCTATGAAGGATGCCCAGGAGGTACCCGCTATATGTGGATCTGACC
TGGGGACCCCTTGCGTAGCTTTACTGCGTAGTGTGGGTTTAAAAAGCGTC
TAGTAGCCTTGTTTTCGGGGGACCGAAAACTTCCACATTATTTTGAGACT
GGCATCATGC

Rabbit picornavirus SEQ ID NO:70

TTCCCCTTCCCCCTTTTCACGGCCCGGCCGCCCGCCGGGTCGTCACCCGT
TCAGACACCTACGGGTTATCTGTTCGGGCTATAAATATGGGCATTCCTCT
TCCCCCTTCCCCTTTTGAAGATGAGTGCGCATATTCTTGACTCCGCCTGG
ATTGGCCGCCCAAGGCGTGAACAAGCAGCTAGGCCACCATGACACTGCGG
TGGTGTCCGAACCCGCGGGTGCCTTCACGGGCACCTGTGGTATGTAGGAC
TCCCACCGTGGTCTTCCCTTTCCCCCTCAATCTTTCCCCCTGGTTCGACT
AACGGGACCAGTGCTGGAACCTGTCCGGTGAACGGTATAGCAGGCCCCCC
CGGCAGAAACACCCGGTGCTTACCCCTTAAGGCTAGCCCCCTTCCATGAA
TTTGGTTGGGGCAACTAGTGGGTGTACAGTTGGCGTGAACCCTCCGGTCT
AGGAGTGCTCTTGCCCAATCCTCTGTGTGTGCCTTGCAGTAGGGACTGGC
AATCCTTCGCGTAGGTGATCCGCTGTGCCATGCCATCCTGGCGACAGGAG
GCCCAGTGTGCGCAACCTACGTCCCTTCTGGGTGCTGCATTGCATTACCT
TTGGAGTAAGCTTGGTGTGCCGAAACCCCAGGGTTTACGTACCACTCGTG
GTGTGAGGAATGTGCCGCAGGTACCCCATCCTTGAGGTGGGATCTGAGCG
GTAGCTAATTGTCTAGCACCACTTTCTTCCTTTTTTCTTTGCTGGTCACG
ATGC

Sudan ebolavirus (SEQ ID NO:71)

TTACCAATGCTTAATCAGTGAGGCACCTATCTCAGCGATCTGTCTATTTC
GTTCATCCATAGTTGCCTGACTCCCCGTCGTGTAGATAACTACGATACGG
GAGGGCTTACCATCTGGCCCCAGTGCATAATATCCTGATGAATTCTATAG
AACTTAGGATTAAGAAAAAATTCATGATGAAGATTAAAACCTTCATCATC
CTTTAAAAAGAGAGCTATTCTTTATCTGAATGTCCTTATTAATGTCTAAG
AGCTATTATTTTGTACCCTCTTAGCCTAGACACTGCCCAGCATATAAGCC
C

Theilers murine encephalomyelitis virus SE ID NO:72)

TTACCAATGCTTAATCAGTGAGGCACCTATCTCAGCGATCTGTCTATTTC
GTTCATCCATAGTTGCCTGACTCCCCGTCGTGTAGATAACTACGATACGG
GAGGGCTTACCATCTGGCCCCAGTGCCTTAAGCGCCTCTGTAGGGAAGCC
AGGAATGTCCAGGAGGTACCCCTTCCGCTCGGAAGGGATCTGACCTGGAG
ACACATCACATGTGCTTTACACCTGTGCTTGTGTTTAAAAATTGTCGCAG
CTTCCCCAAACCAAGTGGTCTTGGTTTTCTCTTTTTATTATATTGTCAAT
C

Insertion of the IRES sequences in place of the CVB3 IRES was performed as follows. PCR was performed with the above-described CVB3 plasmid using primers that introduce at one end a SapI restriction site immediately adjacent to the luciferase ATG start codon and at the other end, near the beginning of the IRES, an AscI site. DNA for nine alternative IRES were synthesized by Twist Biosciences. These were each prepared in the same way. PCR was performed where the appropriate end receives a SapI site with an overhang that will ligate to the SapI cut vector and is immediately adjacent to the 3′ end of the IRES and the other end receives an AscI site. Once digested, the SapI/AscI-cut alternative IRES fragments were ligated to the CVB3 digested SapI/AscI fragment with 10× T4 DNA ligase buffer and T4 DNA ligase at 16 C overnight. The ligated DNA was transformed into NEB5alpha chemically competent cells using the manufacturer's protocol then plates on LB carb (100 ug/ul). Colonies were checked by colony PCR. The IRES region was sequenced and any correct clones had their plasmid prepared before in vitro transcription as described in Example 1. The in vitro transcripts from these constructs along with the CVB3 control were used as described in Example 4 and as shown in FIGS. 18-20.

### Example 4—Sustained Protein Expression from Circular RNAs Containing Diverse IRES Elements Tested in Diverse Cell Lines

This Example demonstrates that circular RNA molecules, created using certain embodiments of the invention described in Example 3, and containing a variety of IRES sequences, are capable, in a variety of tissue types, of expressing their payloads for longer periods than their capped-modified-polyadenylated linear mRNA counterparts.

Circular RNAs generated as described in Example 3, encoding Gaussia luciferase driven by different IRES elements (Tables 3 and 4, Example 3), were transfected using Lipofectamine MessengerMax transfection reagent (ThermoFisher Scientific, LMRNA001) following manufacturer's instructions, into human cell lines of different anatomic origin: HEK293T (kidney), HEPG2 (liver) and HCT116 (colon)

A time-course experiment was performed to monitor protein expression. Cells were seeded at a density of 3*10\hat{ }4 cells/well in 90 ul of Opti-MEM™ (Cat 31985062) and 10 ul of MessengerMax complexed RNA (250 ng) were added per well. Transfection media was removed after 6 hours and cells were placed in their corresponding complete media with serum. Supernatants were collected daily for up to 5 days and Gaussia luciferase activity was assessed by luminescence readout in cell culture supernatants using the Pierce™ Gaussia Luciferase Flash Assay Kit (Cat. 16158) and a PHERAstar plate reader. Blank subtraction was performed and the relative luminescence values at 0.2 seconds were plotted in a time course using GraphPad Prism software.

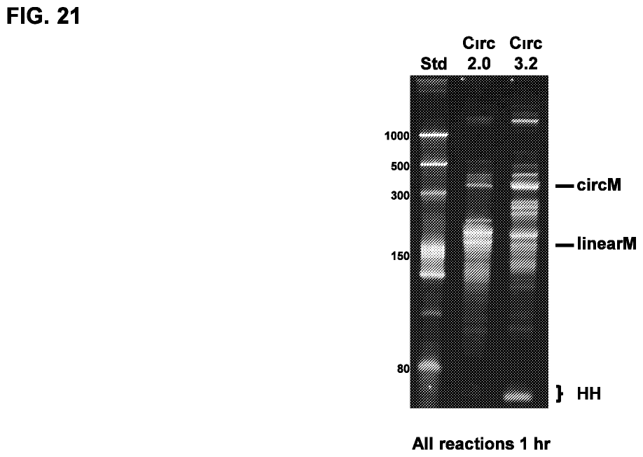

Sustained expression of Gaussia luciferase was detected for all circular RNA transfected cells for 5 days while mRNA expression decayed over time (FIG. 21). This was observed for all cell lines and circular RNAs tested. For at least two of the cell lines, the difference in luminescence is at least one order of magnitude (FIG. 21 left and middle).

### Example 5: Expression of Heterologous Proteins from Circular RNAs

This example demonstrates that alternative coding sequences (CDS) were functional in the CVB3 control construct using a similar method to that used to substitute IRES sequences in place of the CVB3 IRES described in Example 3. This was done as follows. PCR performed on the CVB3 plasmid using primers that introduce at one end a SapI restriction site immediately adjacent to the luciferase ATG start codon and at the other end of the eGFP or mScarlet coding sequence, a SbfI site. PCR performed using eGFP and mScarlet coding sequences with primers that introduce at one end a SapI restriction site immediately adjacent to the eGFP or mScarlet ATG start codon and at the other end of the coding sequence a SbfII site. Once digested, the SapI/SbfI-cut eGFP or mScarlet fragments were ligated to the CVB3 digested SapI/SbfI fragment with 10× T4 DNA ligase buffer and T4 DNA ligase at 16 C overnight. The ligated DNA was transformed into NEB5alpha chemically competent cells using the manufacturer's protocol then plated on LB carb (100 ug/ul). Colonies were checked by colony PCR. The eGFP or mScarlet coding sequence regions were sequenced and any correct clones had their plasmid prepared before in vitro transcription as described in Example 1. The in vitro transcripts from these constructs were used in the experiments, along with the CVB3 control, and a commercial luciferase mRNA control as depicted in Table 5 below.

Once in vitro transcripts were purified, 500 ng of each RNA, including the linear and 5-methoxyU-modified luciferase mRNA control, was prepared with Mirus TransIT mRNA transfection reagent using the manufacturer's protocol. Duplicate wells of a 96 well plate containing 4e4 HEK293T cells in 100 ul media were transfected with 10 ul containing 100 ng RNA. At one day post transfection, the luciferase transfected cells were assayed using the Pierce Gaussia luciferase glow kit following the manufacturer's protocol. At two days post transfection, the eGFP and mScarlet were assayed using a plate reader and eGFP and mScarlet-appropriate filter sets. The results are set forth in Table 5.

Table 5 shows the ability of the circular RNA to express different proteins in HEK293T. Column 1 shows the protein to be expressed. In the case of the luciferase, a circular RNA template and a linear mRNA template fully modified with 5-methoxyU were tested. Column 2 shows expression of mScarlet and column 3 shows expression of eGFP at 2 days post transfection to allow protein to accumulate. Measurements were done with appropriate filter sets with a Biotek Flx800 plate reader and are in relative fluorescence units. Column 4 shows expression of luciferase from circular and linear RNAs at 1 day post transfection. The measurements are done with the plate reader and expressed in relative luminance units. The controls were done by treating cells with Mirus TransIT mRNA transfection reagent only, so controls contain cells and media with no added RNA.

TABLE 5

| | mScarlet | eGFP | Luciferase |
|---|---|---|---|
| mScarlet circular RNA | 1834 | 44 | 464 |
| eGFP circular RNA | 21 | 1253 | 0 |
| Luciferase circular RNA | N/A | N/A | 1440000 |
| Luciferase linear mRNA | 18 | 0 | 3190000 |
| No RNA control | 0 | 0 | 0 |

| | |
|---|---|
| Relative fluorescence | Relative luminance |
| units | units |

The sequences of exemplary reporter sequences are provided below.

### Exemplary Reporter Sequences:

eGFP (SEQ ID NO: 73)

ATGGTGAGCAAGGGCGAGGAGCTGTTCACCGGGGTGGTGCCCATCCTGGT
CGAGCTGGACGGCGACGTAAACGGCCACAAGTTCAGCGTGTCCGGCGAGG
GCGAGGGCGATGCCACCTACGGCAAGCTGACCCTGAAGTTCATCTGCACC
ACCGGCAAGCTGCCCGTGCCCTGGCCCACCCTCGTGACCACCCTGACCTA
CGGCGTGCAGTGCTTCAGCCGCTACCCCGACCACATGAAGCAGCACGACT
TCTTCAAGTCCGCCATGCCCGAAGGCTACGTCCAGGAGCGCACCATCTTC
TTCAAGGACGACGGCAACTACAAGACCCGCGCCGAGGTGAAGTTCGAGGG
CGACACCCTGGTGAACCGCATCGAGCTGAAGGGCATCGACTTCAAGGAGG
ACGGCAACATCCTGGGGCACAAGCTGGAGTACAACTACAACAGCCACAAC
GTCTATATCATGGCCGACAAGCAGAAGAACGGCATCAAGGTGAACTTCAA
GATCCGCCACAACATCGAGGACGGCAGCGTGCAGCTCGCCGACCACTACC
AGCAGAACACCCCCATCGGCGACGGCCCCGTGCTGCTGCCCGACAACCAC
TACCTGAGCACCCAGTCCGCCCTGAGCAAAGACCCCAACGAGAAGCGCGA
TCACATGGTCCTGCTGGAGTTCGTGACCGCCGCCGGGATCACTCTCGGCA
TGGACGAGCTGTACAAGTAA

mScarlet (SEQ ID NO: 74)

ATGGTGAGCAAGGGCGAGGCAGTGATCAAGGAGTTCATGCGGTTCAAGGT
GCACATGGAGGGCTCCATGAACGGCCACGAGTTCGAGATCGAGGGCGAGG
GCGAGGGCCGCCCCTACGAGGGCACCCAGACCGCCAAGCTGAAGGTGACC
AAGGGTGGCCCCCTGCCCTTCTCCTGGGACATCCTGTCCCCTCAGTTCAT
GTACGGCTCCAGGGCCTTCATCAAGCACCCCGCCGACATCCCCGACTACT
ATAAGCAGTCCTTCCCCGAGGGCTTCAAGTGGGAGCGCGTGATGAACTTC
GAGGACGGCGGCGCCGTGACCGTGACCCAGGACACCTCCCTGGAGGACGG
CACCCTGATCTACAAGGTGAAGCTCCGCGGCACCAACTTCCCTCCTGACG
GCCCCGTAATGCAGAAGAAGACAATGGGCTGGGAAGCGTCCACCGAGCGG
TTGTACCCCGAGGACGGCGTGCTGAAGGGCGACATTAAGATGGCCCTGCG
CCTGAAGGACGGCGGCCGCTACCTGGCGGACTTCAAGACCACCTACAAGG
CCAAGAAGCCCGTGCAGATGCCCGGCGCCTACAACGTCGACCGCAAGTTG
GACATCACCTCCCACAACGAGGACTACACCGTGGTGGAACAGTACGAACG
CTCCGAGGGCCGCCACTCCACCGGCGGCATGGACGAGCTGTACAAG

Additional Primer Sequences used in the Examples herein:
PCR of vector oligos

| | |
|---|---|
| IRES Sap | CTCTCTGCTCTTCACATTTTGCTGTATTCAACTTAACAATG (SEQ ID NO: 77) |
| ATG lower | |
| CircRNA | TGTTTTAAGGGGCGCGCCACCA (SEQ ID NO: 78) |
| Asc Rev | |

PCR of IRES Twist fragments

| | |
|---|---|
| Ebola S | CTCTCTGGCGCGCCATAATATCCTGATGAATTCTATAGAACTTAGGAT |
| Asc U | TAAGAAAAAATT (SEQ ID NO: 79) |
| Ebola S | CTCTCTGCTCTTCCCATGGCTTATATGCTGGGCAGTGTC (SEQ ID NO: 80) |
| Fix Sap L | |
| Theilovirus | CTCTCTGGCGCGCCCTTAAGCGCCTCTGTAGGGAAG (SEQ ID NO: 81) |
| Asc U | |
| Theilovirus | CTCTCTGCTCTTCCCATATTGACAATATAATAAAAAGAGAAAACCAA |
| Fix Sap L | GACCACTTG (SEQ ID NO: 82) |
| FGF1 Asc | CTCTCTGGCGCGCCCCAGGCAGTGGGGCC (SEQ ID NO: 83) |
| U | |
| FGF1 Fix | CTCTCTGCTCTTCCCATCATGGGACTCGGATTCAAGGC (SEQ ID NO: 84) |
| Sap L | |
| GF Pico V | CTCTCTGGCGCGCCTGCATGTCATTTCTCCCTTCCCC (SEQ ID NO: 85) |
| Asc U | |
| GF Pico V | CTCTCTGCTCTTCCCATCATATGGCAATCAACTGGCTATACCATG (SEQ ID NO: 86) |
| Fix Sap L | |
| Macaca | CTCTCTGGCGCGCCGGAGGATACTTTGTTTAGCTTTGCAATTCTTAAA |
| Pico V Asc | CT (SEQ ID NO: 87) |
| U | |
| Macaca | CTCTCTGCTCTTCCCATCATGATGCCAGTCTCAAAATAATGTGGAAG |
| Pico V Fix | (SEQ ID NO: 88) |
| Sap L | |
| Rabbit | CTCTCTGGCGCGCCTTCCCCTTCCCCCTTTTCACG (SEQ ID NO: 89) |
| Pico V Asc | |
| U | |
| Rabbit | CTCTCTGCTCTTCCCATCATCGTGACCAGCAAAGAAAAAAGGAA (SEQ ID NO: 90) |
| Pico V Fix | |
| Sap L | |
| EV96 Asc | CTCTCTGGCGCGCCTTAAAACAGCCTGTGGGTTGTTCCC (SEQ ID NO: 91) |
| U | |
| EV96 Fix | CTCTCTGCTCTTCCCATCATTTTGCTGTGTTAAGTGCTAAGATAAAATT |
| Sap L | GAGG (SEQ ID NO: 92) |
| Rat Pico V | CTCTCTGGCGCGCCCCCCCCCTCCCTCCTTTT (SEQ ID NO: 93) |
| Asc U | |
| Rat Pico V | CTCTCTGCTCTTCCCATCATGTCCCAAAACAGCCGATTTCTG (SEQ ID NO: 94) |
| Fix Sap L | |
| EVJ Asc U | CTCTCTGGCGCGCCTTACCAATGCTTAATCAGTGAGGCACC (SEQ ID NO: 95) |
| EVJ Fix | CTCTCTGCTCTTCCCATATTTGGTTGTCTGTGATATGTAAAAGAATTAG |
| Sap L | TATAAACACA (SEQ ID NO: 96) |

PCR of vector oligos

| | |
|---|---|
| Insulator | GACTAACCTGCAGGGGTGTGCCACGA (SEQ ID NO: 97) |
| Sbf upper | |
| IRES Sap | |
| IRES Sap | CTCTCTGCTCTTCACATTTTGCTGTATTCAACTTAACAATG (SEQ ID NO: 98) |
| ATG lower | |

PCR of eGFP and mScarlet (oligos work on both)

| | |
|---|---|
| Sap eGFP | CTCTCTGCTCTTCAATGGTGAGCAAGGGC (SEQ ID NO: 99) |
| upper | |
| Sbf eGFP | CTCTCTCCTGCAGGTTACTTGTACAGCTCGTCCATG (SEQ ID NO: 100) |
| lower | |

Additional Sequences

| SEQ | |
|---|---|
| ID NO: | Sequence |
| 1 | caggggtgtg ccgtggctga caggaaactg tcagccactg tcc |
| 2 | caggggtgtg ccgtggctga cgaaagtcag ccactgtcc |
| 3 | caggggtgtg ccgtggctgg aaacagccac tgtcc |
| 4 | ggtgtgccgt ggcgaaagcc actgtcc |
| 5 | gtcgagtctc c |
| 6 | gtcgagtatc gg |
| 7 | gtcgagtcca atcc |
| 8 | ctcgaggcta gctgtccgtc gagtctccgt tggaattctc gggtgccaag gatgagactc 60 |
| | agaagacaac cagagaaaca cacgttgtgg tatattacct ggtggcgcgc cccgttcaga 120 |
| | gttctacagt ccgacgatcg cggccgcgtc gacggatcca agcttcctgc aggggtgtgc 180 |
| | cgtggctgac aggaaactgt cagccactgt ccgggtcggc atggcatctc cacctcctcg 240 |
| | cggtccgacc tgggcatccg aaggaggacg cacgtccact cggatggcta agggagagcc 300 |
| | atctagagat gtcactagtg ctagacgcgt tgctggcgtt tttccatagg ctccgccccc 360 |
| | ctgacgagca tcacaaaaat cgacgctcaa gtcagaggtg gcgaaacccg acaggactat 420 |
| | aaagatacca ggcgtttccc cctggaagct ccctcgtgcg ctctcctgtt ccgaccctgc 480 |
| | cgcttaccgg atacctgtcc gcctttctcc cttcgggaag cgtggcgctt tctcatagct 540 |
| | cacgctgtag gtatctcagt tcggtgtagg tcgttcgctc caagctgggc tgtgtgcacg 600 |
| | aaccccccgt tcagcccgac cgctgcgcct tatccggtaa ctatcgtctt gagtccaacc 660 |
| | cggtaagaca cgacttatcg ccactggcag cagccactgg taacaggatt agcagagcga 720 |
| | ggtatgtagg cggtgctaca gagttcttga agtggtggcc taactacggc tacactagaa 780 |
| | gaacagtatt tggtatctgc gctctgctga agccagttac cttcggaaaa agagttggta 840 |
| | gctcttgatc cggcaaacaa accaccgctg gtagcggtgg tttttttgtt tgcaagcagc 900 |
| | agattacgcg cagaaaaaaa ggatctcaag aagatccttt gatcttttct acggggtctg 960 |
| | acgctcagtg gaacgaaaac tcacgttaag ggattttggt catgagatta tcaaaaagga 1020 |
| | tcttcaccta gatcctttta aattaaaaat gaagttttaa atcaatctaa agtatatatg 1080 |
| | agtaaacttg gtctgacagt taccaatgct taatcagtga ggcacctatc tcagcgatct 1140 |
| | gtctatttcg ttcatccata gttgcctggc tccccgttgt gtagataact acgatacggg 1200 |
| | agggcttacc atctggcccc agtgctgcaa tgataccgcg agacccacgc tcaccggctc 1260 |
| | cagatttatc agcaataaac cagccagccg gaagggccga gcgcagaagt ggtcctgcaa 1320 |
| | ctttatccgc ctccatccag tctattaatt gttgccggga agctagagta agtagttcgc 1380 |
| | cagttaatag tttgcgcaac gttgttgcca ttgctacagg catcgtggtg tcacgctcgt 1440 |
| | cgtttggtat ggcttcattc agctccggtt cccaacgatc aaggcgagtt acatgatccc 1500 |
| | ccatgttgtg caaaaaagcg gttagctcct tcggtcctcc gatcgttgtc agaagtaagt 1560 |
| | tggccgcagt gttatcactc atggttatgg cagcactgca taattctctt actgtcatgc 1620 |
| | catccgtaag atgcttttct gtgactggtg agtactcaac caagtcattc tgagaatagt 1680 |
| | gtatgcggcg accgagttgc tcttgcccgg cgtcaatacg ggataatacc gcgccacata 1740 |
| | gcagaacttt aaaagtgctc atcattggaa aacgttcttc ggggcgaaaa ctctcaagga 1800 |
| | tcttaccgct gttgagatcc agttcgatgt aacccactcg tgcacccaac tgatcttcag 1860 |
| | catcttttac tttcaccagc gtttctgggt gagcaaaaac aggaaggcaa aatgccgcaa 1920 |
| | aaaagggaat aagggcgaca cggaaatgtt gaatactcat actcttcctt tttcaatatt 1980 |
| | attgaagcat ttatcagggt tattgtctca tgagcggata catatttgaa tgtatttaga 2040 |
| | aaaataaaca aataggggtt ccgcgggtac cactagtgac atc 2083 |
| 9 | ctctctcgag taatacgact cactataggg tgtccgtcga gtctccgttg ga |
| 10 | acggagactc gacggacagt ggctgacagt ttcctgtcag ccacggcaca cccctg |
| 11 | agtgcgagct cgagccgtta cctcgacctg atgagctcca aaaagagcga aacctattag 60 |
| | gtcgtcgagt actgggttgg aattctcggg tgccaaggat agtactcaga agacaaccag 120 |
| | agaaacacac gttgtggtat attacctggt ggcgcgcctg aggtt 165 |
| 12 | taatacgact cactatagga gtgcgagctc gagccgttac ctcgacctga tgagctccaa 60 |
| | aaagagcgaa acctattagg tcgtcgagta ctgggttgga attctcgggt gccaaggata 120 |
| | gtactcagaa gacaaccaga gaaacacacg ttgtggtata ttacctggtg gcgcgccccg 180 |
| | ttcagagttc tacagtccga cgatcgcggc cgcgtcgacg gatccaagct tcctgcaggg 240 |
| | gtgtgccgtg gctgacagga aactgtcagc cactgtccgt cgagtact 288 |
| 13 | taatacgact cactatagga gtgcgagctc gagccgt |
| 14 | agtactcgac ggacagtggc tgacagtttc ctgtcagcca cggcacaccc ctg |
| 15 | taatacgact cactatagga gtgcgagctc gagccgttac ctcgacctga tgagctccaa 60 |
| | aaagagcgaa acctattagg tcgtcgagta ctgggttgga attctcgggt gccaaggata 120 |
| | gtactcagaa gacaaccaga gaaacacacg ttgtggtata ttacctggtg gcgcgccccg 180 |
| | ttcagagttc tacagtccga cgatcgcggc cgcgtcgacg gatccaagct tcctgcaggg 240 |
| | gtgtgccgtg gctgacagga aactgtcagc cactgtccgt cgagtctccg t 291 |
| 16 | taatacgact cactatagga gtgcgagctc gagccgttac ctcgacctga tgagctccaa 60 |
| | aaagagcgaa acctattagg tcgtcgagta ctgggttgga attctcgggt gccaaggatg 120 |
| | agactcagaa gacaaccaga gaaacacacg ttgtggtata ttacctggtg gcgcgccccg 180 |
| | ttcagagttc tacagtccga cgatcgcggc cgcgtcgacg gatccaagct tcctgcaggg 240 |
| | gtgtgccgtg gctgacagga aactgtcagc cactgtccgt cgagtctccg t 291 |
| 17 | agtgcgagct cgaggagact cagaagacaa acggcgaaac acaccttgtg tggtatatta 60 |
| | cccgttggag attccagagg attggttacc tatctcccat gcccatgtcg gcattgtccg 120 |
| | tcgagtctcc gttggaattc tcgggtgcca aggatgagac tcagaagaca accagagaaa 180 |
| | cacac 185 |
| 18 | taatacgact cactatagga gtgcgagctc gaggaga |
| 19 | taatacgact cactatagga gtgcgagctc gaggagactc agaagacaaa cggcgaaaca 60 |
| | caccttgtgt ggtatattac ccgttggaga ttccagagga ttggttacct atctcccatg 120 |
| | cccatgtcgg cattgtccgt cgagtctccg ttggaattct cgggtgccaa ggatgagact 180 |
| | cagaagacaa ccagagaaac acacgttgtg gtatattacc tggtggcgcg ccccgttcag 240 |
| | agttctacag tccgacgatc gcggccgcgt cgacggatcc aagcttcctg caggggtgtg 300 |
| | ccgtggctga caggaaactg tcagccactg tccgtcgagt ctccgt 346 |
| 20 | taatacgact cactatagga gtgcgagctc gagggatgta cctcgacctg atgagtccca 60 |
| | aataggacga aacgccgaaa ggcgtcgtcg agtccaatcc gttggaattc tcgggtgcca 120 |
| | aggattggac tcagaagaca accagagaaa cacacgttgt ggtatattac ctggtggcgc 180 |
| | gccccgttca gagttctaca gtccgacgat cgcggccgcg tcgacggatc caagcttcct 240 |
| | gcaggggtgt gccgtggctg acaggaaact gtcagccact gtccgtcgag tctccgt 297 |
| 21 | agtgcgagct cgagggatgt acctcgacct gatgagtccc aaataggacg aaacgccgaa 60 |
| | aggcgtcgtc gagtccaatc cgttggaatt ctcgggtgcc aaggattgga ctcagaagac 120 |
| | aaccagagaa acacacgttg tggtatatta cctggtggcg cgcctgaggt t 171 |
| 22 | taatacgact cactatagga gtgcgagctc gagggat |
| 23 | taatacgact cactatagga gtgcgagctc gagggatgta cctcgacctg atgagtccca 60 |
| | aataggacga aacgccgaaa ggcgtcgtcg agtccaatcc gttggaattc tcgggtgcca 120 |
| | aggatgagac tcagaagaca accagagaaa cacacgttgt ggtatattac ctggtggcgc 180 |
| | gccccgttca gagttctaca gtccgacgat cgcggccgcg tcgacggatc caagcttcct 240 |
| | gcaggggtgt gccgtggctg acaggaaact gtcagccact gtccgtcgag tctccgt 297 |
| 24 | tatatagatc tccgttacct cgacctgatg agctccaaaa agagcgaaac ctattaggtc 60 |
| | gtcgagtact gggttggaat tctcgggtgc caaggatagt actcagaaga caaccagaga 120 |
| | aacacacgtt gtggtatatt acctggtggc gcgccccgtt cagagttcta cagtccgacg 180 |
| | atcgcggccg cgtcgacgga tccaagcttc ctgcaggggt gtgccgtggc tgacaggaaa 240 |
| | ctgtcagcca ctgtccgggt cggcatggca tctccacctc ctcgcggtcc gacctgggca 300 |
| | tccgaaggag gacgcacgtc cactcggatg gctaagggag agccatctag actgaggttg 360 |
| | atgtcactag tgctagacgc gttgctggcg tttttccata ggctccgccc ccctgacgag 420 |
| | catcacaaaa atcgacgctc aagtcagagg tggcgaaacc cgacaggact ataaagatac 480 |
| | caggcgtttc cccctggaag ctccctcgtg cgctctcctg ttccgaccct gccgcttacc 540 |
| | ggatacctgt ccgcctttct cccttcggga agcgtggcgc tttctcatag ctcacgctgt 600 |
| | aggtatctca gttcggtgta ggtcgttcgc tccaagctgg gctgtgtgca cgaacccccc 660 |
| | gttcagcccg accgctgcgc cttatccggt aactatcgtc ttgagtccaa cccggtaaga 720 |
| | cacgacttat cgccactggc agcagccact ggtaacagga ttagcagagc gaggtatgta 780 |
| | ggcggtgcta cagagttctt gaagtggtgg cctaactacg gctacactag aagaacagta 840 |
| | tttggtatct gcgctctgct gaagccagtt accttcggaa aaagagttgg tagctcttga 900 |
| | tccggcaaac aaaccaccgc tggtagcggt ggtttttttg tttgcaagca gcagattacg 960 |
| | cgcagaaaaa aaggatctca agaagatcct ttgatctttt ctacggggtc tgacgctcag 1020 |
| | tggaacgaaa actcacgtta agggattttg gtcatgagat tatcaaaaag gatcttcacc 1080 |
| | tagatccttt taaattaaaa atgaagtttt aaatcaatct aaagtatata tgagtaaact 1140 |
| | tggtctgaca gttaccaatg cttaatcagt gaggcaccta tctcagcgat ctgtctattt 1200 |
| | cgttcatcca tagttgcctg gctccccgtt gtgtagataa ctacgatacg ggagggctta 1260 |
| | ccatctggcc ccagtgctgc aatgataccg cgagacccac gctcaccggc tccagattta 1320 |
| | tcagcaataa accagccagc cggaagggcc gagcgcagaa gtggtcctgc aactttatcc 1380 |
| | gcctccatcc agtctattaa ttgttgccgg gaagctagag taagtagttc gccagttaat 1440 |
| | agtttgcgca acgttgttgc cattgctaca ggcatcgtgg tgtcacgctc gtcgtttggt 1500 |
| | atggcttcat tcagctccgg ttcccaacga tcaaggcgag ttacatgatc ccccatgttg 1560 |
| | tgcaaaaaag cggttagctc cttcggtcct ccgatcgttg tcagaagtaa gttggccgca 1620 |
| | gtgttatcac tcatggttat ggcagcactg cataattctc ttactgtcat gccatccgta 1680 |
| | agatgctttt ctgtgactgg tgagtactca accaagtcat tctgagaata gtgtatgcgg 1740 |
| | cgaccgagtt gctcttgccc ggcgtcaata cgggataata ccgcgccaca tagcagaact 1800 |
| | ttaaaagtgc tcatcattgg aaaacgttct tcggggcgaa aactctcaag gatcttaccg 1860 |
| | ctgttgagat ccagttcgat gtaacccact cgtgcaccca actgatcttc agcatctttt 1920 |
| | actttcacca gcgtttctgg gtgagcaaaa acaggaaggc aaaatgccgc aaaaaaggga 1980 |
| | ataagggcga cacggaaatg ttgaatactc atactcttcc tttttcaata ttattgaagc 2040 |
| | atttatcagg gttattgtct catgagcgga tacatatttg aatgtattta gaaaaataaa 2100 |
| | caaatagggg ttccgcgggt accactagtg acatc 2135 |
| 25 | taatacgact cactatagga gtgcgagctc gagccgttac ctcgacctga tgagctccaa 60 |
| | aaagagcgaa acctattagg tcgtcgagta ctgggttgga attctcgggt gccaaggata 120 |
| | gtactcagaa gacaaccaga gaaacacacg ttgtggtata ttacctggtg gcgcgccccg 180 |
| | ttcagagttc tacagtccga cgatcgcggc cgcgtcgacg gatccaagct tcctgcaggg 240 |
| | gtgtgccgtg gctgacagga aactgtcagc cactgtccgt cgagtact 288 |
| 26 | tatatagatc tccgttacct cgacctgatg ag |
| 27 | cgtctagatg gctctccctt agccatccga gtggacgtg |
| 28 | actagtgaca tcatgtcgat atcctatgcg gcatcagagc agattgtact gagagtgcac 60 |
| | catatgcggt gtgaaatacc gcacagatgc gtaaggagaa aataccgcat caggcgccat 120 |
| | tcgccattca ggctgcgcaa ctgttgggaa gggcgatcgg tgcgggcctc ttcgctatta 180 |
| | cgccagctgg cgaaaggggg atgtgctgca aggcgattaa gttgggtaac gccagggttt 240 |
| | tcccagtcac gacgttgtaa aacgacggcc agtgaattcg agctcggtac ccggggatcc 300 |
| | tctagagtcg acctgcaggc atgcaagctt ggcgtaatca tggtcatagc tgtttcctgt 360 |
| | gtgaaattgt tatccgctca caattccaca caacatacga gccggaagca taaagtgtaa 420 |
| | agcctggggt gcctaatgag tgagctaact cacattaatt gcgttgcgct cactgcccgc 480 |
| | tttccagtcg ggaaacctgt cgtgccagcg atatcgacat gatgtcacta gtgctagacg 540 |
| | cgttgctggc gtttttccat aggctccgcc cccctgacga gcatcacaaa aatcgacgct 600 |
| | caagtcagag gtggcgaaac ccgacaggac tataaagata ccaggcgttt ccccctggaa 660 |
| | gctccctcgt gcgctctcct gttccgaccc tgccgcttac cggatacctg tccgcctttc 720 |
| | tcccttcggg aagcgtggcg ctttctcata gctcacgctg taggtatctc agttcggtgt 780 |
| | aggtcgttcg ctccaagctg ggctgtgtgc acgaaccccc cgttcagccc gaccgctgcg 840 |
| | ccttatccgg taactatcgt cttgagtcca acccggtaag acacgactta tcgccactgg 900 |
| | cagcagccac tggtaacagg attagcagag cgaggtatgt aggcggtgct acagagttct 960 |
| | tgaagtggtg gcctaactac ggctacacta gaagaacagt atttggtatc tgcgctctgc 1020 |
| | tgaagccagt taccttcgga aaaagagttg gtagctcttg atccggcaaa caaaccaccg 1080 |
| | ctggtagcgg tggttttttt gtttgcaagc agcagattac gcgcagaaaa aaaggatctc 1140 |
| | aagaagatcc tttgatcttt tctacggggt ctgacgctca gtggaacgaa aactcacgtt 1200 |
| | aagggatttt ggtcatgaga ttatcaaaaa ggatcttcac ctagatcctt ttaaattaaa 1260 |
| | aatgaagttt taaatcaatc taaagtatat atgagtaaac ttggtctgac agttaccaat 1320 |
| | gcttaatcag tgaggcacct atctcagcga tctgtctatt tcgttcatcc atagttgcct 1380 |
| | ggctccccgt tgtgtagata actacgatac gggagggctt accatctggc cccagtgctg 1440 |
| | caatgatacc gcgagaccca cgctcaccgg ctccagattt atcagcaata aaccagccag 1500 |
| | ccggaagggc cgagcgcaga agtggtcctg caactttatc cgcctccatc cagtctatta 1560 |
| | attgttgccg ggaagctaga gtaagtagtt cgccagttaa tagtttgcgc aacgttgttg 1620 |
| | ccattgctac aggcatcgtg gtgtcacgct cgtcgtttgg tatggcttca ttcagctccg 1680 |
| | gttcccaacg atcaaggcga gttacatgat cccccatgtt gtgcaaaaaa gcggttagct 1740 |
| | ccttcggtcc tccgatcgtt gtcagaagta agttggccgc agtgttatca ctcatggtta 1800 |
| | tggcagcact gcataattct cttactgtca tgccatccgt aagatgcttt tctgtgactg 1860 |
| | gtgagtactc aaccaagtca ttctgagaat agtgtatgcg gcgaccgagt tgctcttgcc 1920 |
| | cggcgtcaat acgggataat accgcgccac atagcagaac tttaaaagtg ctcatcattg 1980 |
| | gaaaacgttc ttcggggcga aaactctcaa ggatcttacc gctgttgaga tccagttcga 2040 |
| | tgtaacccac tcgtgcaccc aactgatctt cagcatcttt tactttcacc agcgtttctg 2100 |
| | ggtgagcaaa aacaggaagg caaaatgccg caaaaaaggg aataagggcg acacggaaat 2160 |
| | gttgaatact catactcttc ctttttcaat attattgaag catttatcag ggttattgtc 2220 |
| | tcatgagcgg atacatattt gaatgtattt agaaaaataa acaaataggg gttccgcggg 2280 |
| | tacc 2284 |
| 29 | taatacgact cactatagga gtgcgagctc gagccgttac ctcgacctga tgagctccaa 60 |
| | aaagagcgaa acctattagg tcgtcgagta ctgggttgga attctcgggt gccaaggata 120 |
| | gtactcagaa accgaccaga gaaacacacg ttgtggtata ttacctggtg gcgcgcccgt 180 |
| | tcagagttct acagtccgac gatcgcggcc gcgtcgacgg atccaagctt cctgcagggg 240 |
| | tgtgccgtgg ctgacaggaa actgtcagcc accggtagtc gagtctccgt tctagactga 300 |
| | ggttgatgtc actagtgcta gacgcgttgc tggcgttttt ccataggctc cgcccccctg 360 |
| | acgagcatca caaaaatcga cgctcaagtc agaggtggat gtcactagtg ctagacgcgt 420 |
| | tgctggcgtt tttccatagg ctccgccccc ctgacgagca tcacaaaaat cgacgctcaa 480 |
| | gtcagaggtg gcgaaacccg acaggactat aaagatacca ggcgtttccc cctggaagct 540 |
| | ccctcgtgcg ctctcctgtt ccgaccctgc cgcttaccgg atacctgtcc gcctttctcc 600 |
| | cttcgggaag cgtggcgctt tctcatagct cacgctgtag gtatctcagt tcggtgtagg 660 |
| | tcgttcgctc caagctgggc tgtgtgcacg aaccccccgt tcagcccgac cgctgcgcct 720 |
| | tatccggtaa ctatcgtctt gagtccaacc cggtaagaca cgacttatcg ccactggcag 780 |
| | cagccactgg taacaggatt agcagagcga ggtatgtagg cggtgctaca gagttcttga 840 |
| | agtggtggcc taactacggc tacactagaa gaacagtatt tggtatctgc gctctgctga 900 |
| | agccagttac cttcggaaaa agagttggta gctcttgatc cggcaaacaa accaccgctg 960 |
| | gtagcggtgg tttttttgtt tgcaagcagc agattacgcg cagaaaaaaa ggatctcaag 1020 |
| | aagatccttt gatcttttct acggggtctg acgctcagtg gaacgaaaac tcacgttaag 1080 |
| | ggattttggt catgagatta tcaaaaagga tcttcaccta gatcctttta aattaaaaat 1140 |
| | gaagttttaa atcaatctaa agtatatatg agtaaacttg gtctgacagt taccaatgct 1200 |
| | taatcagtga ggcacctatc tcagcgatct gtctatttcg ttcatccata gttgcctggc 1260 |
| | tccccgttgt gtagataact acgatacggg agggcttacc atctggcccc agtgctgcaa 1320 |
| | tgataccgcg agacccacgc tcaccggctc cagatttatc agcaataaac cagccagccg 1380 |
| | gaagggccga gcgcagaagt ggtcctgcaa ctttatccgc ctccatccag tctattaatt 1440 |
| | gttgccggga agctagagta agtagttcgc cagttaatag tttgcgcaac gttgttgcca 1500 |
| | ttgctacagg catcgtggtg tcacgctcgt cgtttggtat ggcttcattc agctccggtt 1560 |
| | cccaacgatc aaggcgagtt acatgatccc ccatgttgtg caaaaaagcg gttagctcct 1620 |
| | tcggtcctcc gatcgttgtc agaagtaagt tggccgcagt gttatcactc atggttatgg 1680 |
| | cagcactgca taattctctt actgtcatgc catccgtaag atgcttttct gtgactggtg 1740 |
| | agtactcaac caagtcattc tgagaatagt gtatgcggcg accgagttgc tcttgcccgg 1800 |
| | cgtcaatacg ggataatacc gcgccacata gcagaacttt aaaagtgctc atcattggaa 1860 |
| | aacgttcttc ggggcgaaaa ctctcaagga tcttaccgct gttgagatcc agttcgatgt 1920 |
| | aacccactcg tgcacccaac tgatcttcag catcttttac tttcaccagc gtttctgggt 1980 |
| | gagcaaaaac aggaaggcaa aatgccgcaa aaaagggaat aagggcgaca cggaaatgtt 2040 |
| | gaatactcat actcttcctt tttcaatatt attgaagcat ttatcagggt tattgtctca 2100 |
| | tgagcggata catatttgaa tgtatttaga aaaataaaca aataggggtt ccgcgggtac 2160 |
| | cactagtgac atc 2173 |
| 30 | ctcgaggcta gctgtccgtc gagtctccgt tggaattctc gggtgccaag gatgagactc 60 |
| | agaaaccgac cagagaaaca cacgttgtgg tatattacct ggtggcgcgc cccgttcaga 120 |
| | gttctacagt ccgacgatcg cggccgcgtc gacggatcca agcttcctgc agggctgntc 180 |
| | cgtggctgac aggaaactgt cagccaccgg tagtcgagtc tccgttctag agatgtcact 240 |
| | agtgctagac gcgttgctgg cgtttttcca taggctccgc ccccctgacg agcatcacaa 300 |
| | aaatcgacgc tcaagtcaga ggtggcgaaa cccgacagga ctataaagat accaggcgtt 360 |
| | tccccctgga agctccctcg tgcgctctcc tgttccgacc ctgccgctta ccggatacct 420 |
| | gtccgccttt ctcccttcgg gaagcgtggc gctttctcat agctcacgct gtaggtatct 480 |
| | cagttcggtg taggtcgttc gctccaagct gggctgtgtg cacgaacccc ccgttcagcc 540 |
| | cgaccgctgc gccttatccg gtaactatcg tcttgagtcc aacccggtaa gacacgactt 600 |
| | atcgccactg gcagcagcca ctggtaacag gattagcaga gcgaggtatg taggcggtgc 660 |
| | tacagagttc ttgaagtggt ggcctaacta cggctacact agaagaacag tatttggtat 720 |
| | ctgcgctctg ctgaagccag ttaccttcgg aaaaagagtt ggtagctctt gatccggcaa 780 |
| | acaaaccacc gctggtagcg gtggtttttt tgtttgcaag cagcagatta cgcgcagaaa 840 |
| | aaaaggatct caagaagatc ctttgatctt ttctacgggg tctgacgctc agtggaacga 900 |
| | aaactcacgt taagggattt tggtcatgag attatcaaaa aggatcttca cctagatcct 960 |
| | tttaaattaa aaatgaagtt ttaaatcaat ctaaagtata tatgagtaaa cttggtctga 1020 |
| | cagttaccaa tgcttaatca gtgaggcacc tatctcagcg atctgtctat ttcgttcatc 1080 |
| | catagttgcc tggctccccg ttgtgtagat aactacgata cgggagggct taccatctgg 1140 |
| | ccccagtgct gcaatgatac cgcgagaccc acgctcaccg gctccagatt tatcagcaat 1200 |
| | aaaccagcca gccggaaggg ccgagcgcag aagtggtcct gcaactttat ccgcctccat 1260 |
| | ccagtctatt aattgttgcc gggaagctag agtaagtagt tcgccagtta atagtttgcg 1320 |
| | caacgttgtt gccattgcta caggcatcgt ggtgtcacgc tcgtcgtttg gtatggcttc 1380 |
| | attcagctcc ggttcccaac gatcaaggcg agttacatga tcccccatgt tgtgcaaaaa 1440 |
| | agcggttagc tccttcggtc ctccgatcgt tgtcagaagt aagttggccg cagtgttatc 1500 |
| | actcatggtt atggcagcac tgcataattc tcttactgtc atgccatccg taagatgctt 1560 |
| | ttctgtgact ggtgagtact caaccaagtc attctgagaa tagtgtatgc ggcgaccgag 1620 |
| | ttgctcttgc ccggcgtcaa tacgggataa taccgcgcca catagcagaa ctttaaaagt 1680 |
| | gctcatcatt ggaaaacgtt cttcggggcg aaaactctca aggatcttac cgctgttgag 1740 |
| | atccagttcg atgtaaccca ctcgtgcacc caactgatct tcagcatctt ttactttcac 1800 |
| | cagcgtttct gggtgagcaa aaacaggaag gcaaaatgcc gcaaaaaagg gaataagggc 1860 |
| | gacacggaaa tgttgaatac tcatactctt cctttttcaa tattattgaa gcatttatca 1920 |
| | gggttattgt ctcatgagcg gatacatatt tgaatgtatt tagaaaaata aacaaatagg 1980 |
| | ggttccgcgg gtaccactag tgacatc 2007 |
| 31 | atcggccggc ccgcggaacc cctatttgtt tatttttcta aatac |
| 32 | gggttggaat tctcgggtgc caaggatagt actcagaaac cgac |
| 33 | cacctctgac ttgagcgtcg attt |
| 34 | taatacgact cactatagga gatctccgtt acctcgacct gatgagctcc aaaaagagcg 60 |
| | aaacctatta ggtcgtcgag tactgggttg gaattctcgg gtgccaagga tagtactcag 120 |
| | aagacaacca gagaaacaca cgttgtggta tattacctgg tggcgcgccc cgttcagagt 180 |
| | tctacagtcc gacgatcgcg gccgcgtcga cggatccaag cttcctgcag gggtgtgccg 240 |
| | tggcgaaagc cactgtccgt cgagtactgg 270 |
| 35 | taatacgact cactatagga gatctccgtt acctcgacct gatgag |
| 36 | ccagtactcg acggacagtg gctttcgcca cggcacacc |
| 37 | taatacgact cactatagga gatctccgtt acctcgacct gatgagctcc aaaaagagcg 60 |
| | aaacctatta ggtcgtcgag tactgggttg gaattctcgg gtgccaagga tagtactcag 120 |
| | aagacaacca gagaaacaca cgttgtggta tattacctgg tggcgcgccc cgttcagagt 180 |
| | tctacagtcc gacgatcgcg gccgcgtcga cggatccaag cttcctgcag gggtgtgccg 240 |
| | tggctggaaa cagccactgt ccgtcgagta ctgg 274 |
| 38 | ccagtactcg acggacagtg gctgtttcca gccacggcac acccctg |
| 39 | taatacgact cactatagga gatctccgtt acctcgacct gatgagctcc aaaaagagcg 60 |
| | aaacctatta ggtcgtcgag tactgggttg gaattctcgg gtgccaagga tagtactcag 120 |
| | aagacaacca gagaaacaca cgttgtggta tattacctgg tggcgcgccc cgttcagagt 180 |
| | tctacagtcc gacgatcgcg gccgcgtcga cggatccaag cttcctgcag gggtgtgccg 240 |
| | tggctgacga aagtcagcca ctgtccgtcg agtactgg 278 |
| 40 | ccagtactcg acggacagtg gctgactttc gtcagccacg gcacacccct g |
| 41 | taatacgact cactatagga gatctccgtt acctcgacct gatgagctcc aaaaagagcg 60 |
| | aaacctatta ggtcgtcgag tactgggttg gaattctcgg gtgccaagga tagtactcag 120 |
| | aagacaacca gagaaacaca cgttgtggta tattacctgg tggcgcgccc cgttcagagt 180 |
| | tctacagtcc gacgatcgcg gccgcgtcga cggatccaag cttcctgcag gggtgtgccg 240 |
| | tggctgacag gaaactgtca gccactgtcc gtcgagtact gg 282 |
| 42 | ccagtactcg acggacagtg gctgacagtt tcctgtcagc cacggcacac ccctg |
| 43 | agtgcgagcc tgcaggggtg tgccgtggct gacaggaaac tgtcagccac tgtccgtcga 60 |
| | gtctccgtat gagactcaga agacaaacgg cgaaacacac cttgtgtggt atattacccg 120 |
| | tttctagact gaggtt |
| 44 | aacctcagtc tagaaacggg taa |
| 45 | taatacgact cactatagga gatctccgtt acctcgacct gatgagctcc aaaaagagcg 60 |
| | aaacctatta ggtcgtcgag tactgggttg gaattctcgg gtgccaagga tagtactcag 120 |
| | aagacaacca gagaaacaca cgttgtggta tattacctgg tggcgcgccc cgttcagagt 180 |
| | tctacagtcc gacgatcgcg gccgcgtcga cggatccaag cttcctgcag gggtgtgccg 240 |
| | tggctgacag gaaactgtca gccactgtcc gtcgagtctc cgtatgagac tcagaagaca 300 |
| | aacggcgaaa cacaccttgt gtggtatatt acccgtttct agactgaggt t 351 |
| 46 | agtgcgagcc tgcaggggtg tgccgtggct gacaggaaac tgtcagccac cggtcctggt 60 |
| | atccaatccg aaaggatgta cctcgacctg atgagtccca aataggacga aaccggtcta 120 |
| | gactgaggtt |
| 47 | aacctcagtc tagaccggtt tc |
| 48 | taatacgact cactatagga gtgcgagctc gagccgttac ctcgacctga tgagctccaa 60 |
| | aaagagcgaa acctattagg tcgtcgagta ctgggttgga attctcgggt gccaaggata 120 |
| | gtactcagaa accgaccaga gaaacacacg ttgtggtata ttacctggtg gcgcgccccg 180 |
| | ttcagagttc tacagtccga cgatcgcggc cgcgtcgacg gatccaagct tcctgcaggg 240 |
| | gtgtgccgtg gctgacagga aactgtcagc caccggtcct ggtatccaat ccgaaaggat 300 |
| | gtacctcgac ctgatgagtc ccaaatagga cgaaaccggt ctagactgag gtt 353 |
| 49 | agtactcgac gaccggtggc tgacagtttc ctgtcagcca cggcacaccc ctg |
| 50 | taatacgact cactataggg ctcgaggcta gccggtcgtc gagtctccgt tggaattctc 60 |
| | gggtgccaag gatgagactc agaaaccgac cagagaaaca cacgttgtgg tatattacct 120 |
| | ggtggcgcgc ccgttcagag ttctacagtc cgacgatcgc ggccgcgtcg acggatccaa 180 |
| | gcttcctgca ggggtgtgcc gtggctgaca ggaaactgtc agccaccggt ctgtgctaag 240 |
| | cacactgatg agtctctgaa atgagacgaa accggtctag actgaggtt 289 |
| 51 | agtgcgagcc tgcaggggtg tgccgtggct gacaggaaac tgtcagccac cggtctgtgc 60 |
| | taagcacact gatgagtctc tgaaatgaga cgaaaccggt ctagactgag gtt 113 |
| 52 | taatacgact cactataggg ctcgaggcta gccggtcgtc gagtc |
| 53 | taatacgact cactatagga gtgcgagctc gagccgttac ctcgacctga tgagctccaa 60 |
| | aaagagcgaa acctattagg tcgtcgagta ctgggttgga attctcgggt gccaaggata 120 |
| | gtactcagaa accgaccaga gaaacacacg ttgtggtata ttacctggtg gcgcgccccg 180 |
| | ttcagagttc tacagtccga cgatcgcggc cgcgtcgacg gatccaagct tcctgcaggg 240 |
| | gtgtgccgtg gctgacagga aactgtcagc caccggtctg tgctaagcac actgatgagt 300 |
| | ctctgaaatg agacgaaacc ggtctagact gaggtt 336 |
| 54 | tatatagatc tccgttacct cgacctgatg agctccaaaa agagcgaaac ctattaggtc 60 |
| | gtcgagtact gggttggaat tctcgggtgc caaggatagt actcagaaga caaccagaga 120 |
| | aacacacgtt gtggtatatt acctggtggc gcgccccgtt cagagttcta cagtccgacg 180 |
| | atcgcggccg cgtcgacgga tccaagcttc ctgcaggggt gtgccgtggc tgacaggaaa 240 |
| | ctgtcagcca ctgtccgtcg agtacgtcta gaatatagat gtcactagtg ctagacgcgt 300 |
| | tgctggcgtt tttccatagg ctccgccccc ctgacgagca tcacaaaaat cgacgctcaa 360 |
| | gtcagaggtg gcgaaacccg acaggactat aaagatacca ggcgtttccc cctggaagct 420 |
| | ccctcgtgcg ctctcctgtt ccgaccctgc cgcttaccgg atacctgtcc gcctttctcc 480 |
| | cttcgggaag cgtggcgctt tctcatagct cacgctgtag gtatctcagt tcggtgtagg 540 |
| | tcgttcgctc caagctgggc tgtgtgcacg aaccccccgt tcagcccgac cgctgcgcct 600 |
| | tatccggtaa ctatcgtctt gagtccaacc cggtaagaca cgacttatcg ccactggcag 660 |
| | cagccactgg taacaggatt agcagagcga ggtatgtagg cggtgctaca gagttcttga 720 |
| | agtggtggcc taactacggc tacactagaa gaacagtatt tggtatctgc gctctgctga 780 |
| | agccagttac cttcggaaaa agagttggta gctcttgatc cggcaaacaa accaccgctg 840 |
| | gtagcggtgg tttttttgtt tgcaagcagc agattacgcg cagaaaaaaa ggatctcaag 900 |
| | aagatccttt gatcttttct acggggtctg acgctcagtg gaacgaaaac tcacgttaag 960 |
| | ggattttggt catgagatta tcaaaaagga tcttcaccta gatcctttta aattaaaaat 1020 |
| | gaagttttaa atcaatctaa agtatatatg agtaaacttg gtctgacagt taccaatgct 1080 |
| | taatcagtga ggcacctatc tcagcgatct gtctatttcg ttcatccata gttgcctggc 1140 |
| | tccccgttgt gtagataact acgatacggg agggcttacc atctggcccc agtgctgcaa 1200 |
| | tgataccgcg agacccacgc tcaccggctc cagatttatc agcaataaac cagccagccg 1260 |
| | gaagggccga gcgcagaagt ggtcctgcaa ctttatccgc ctccatccag tctattaatt 1320 |
| | gttgccggga agctagagta agtagttcgc cagttaatag tttgcgcaac gttgttgcca 1380 |
| | ttgctacagg catcgtggtg tcacgctcgt cgtttggtat ggcttcattc agctccggtt 1440 |
| | cccaacgatc aaggcgagtt acatgatccc ccatgttgtg caaaaaagcg gttagctcct 1500 |
| | tcggtcctcc gatcgttgtc agaagtaagt tggccgcagt gttatcactc atggttatgg 1560 |
| | cagcactgca taattctctt actgtcatgc catccgtaag atgcttttct gtgactggtg 1620 |
| | agtactcaac caagtcattc tgagaatagt gtatgcggcg accgagttgc tcttgcccgg 1680 |
| | cgtcaatacg ggataatacc gcgccacata gcagaacttt aaaagtgctc atcattggaa 1740 |
| | aacgttcttc ggggcgaaaa ctctcaagga tcttaccgct gttgagatcc agttcgatgt 1800 |
| | aacccactcg tgcacccaac tgatcttcag catcttttac tttcaccagc gtttctgggt 1860 |
| | gagcaaaaac aggaaggcaa aatgccgcaa aaaagggaat aagggcgaca cggaaatgtt 1920 |
| | gaatactcat actcttcctt tttcaatatt attgaagcat ttatcagggt tattgtctca 1980 |
| | tgagcggata catatttgaa tgtatttaga aaaataaaca aataggggtt ccgcgggtac 2040 |
| | cactagtgac atc 2053 |
| 55 | tatattctag acgtactcga cggacagtgg ctgacagttt cctgtcagcc acggcacacc 60 |
| | cctg 64 |
| 56 | tatatagatc tccgttacct cgacctgatg agctccaaaa agagcgaaac ctattaggtc 60 |
| | gtcgagtact gggttggaat tctcgggtgc caaggatagt actcagaaac cgaccagaga 120 |
| | aacacacgtt gtggtatatt acctggtggc gcgccccgtt cagagttcta cagtccgacg 180 |
| | atcgcggccg cgtcgacgga tccaagcttc ctgcaggggt gtgccgtggc tgacaggaaa 240 |
| | ctgtcagcca ccggtcctgg tatccaatcc gaaaggatgt acctcgacct gatgagtccc 300 |
| | aaataggacg aaaccggtct agactgaggt tgatgtcact agtgctagac gcgttgctgg 360 |
| | cgtttttcca taggctccgc ccccctgacg agcatcacaa aaatcgacgc tcaagtcaga 420 |
| | ggtggcgaaa cccgacagga ctataaagat accaggcgtt tccccctgga agctccctcg 480 |
| | tgcgctctcc tgttccgacc ctgccgctta ccggatacct gtccgccttt ctcccttcgg 540 |
| | gaagcgtggc gctttctcat agctcacgct gtaggtatct cagttcggtg taggtcgttc 600 |
| | gctccaagct gggctgtgtg cacgaacccc ccgttcagcc cgaccgctgc gccttatccg 660 |
| | gtaactatcg tcttgagtcc aacccggtaa gacacgactt atcgccactg gcagcagcca 720 |
| | ctggtaacag gattagcaga gcgaggtatg taggcggtgc tacagagttc ttgaagtggt 780 |
| | ggcctaacta cggctacact agaagaacag tatttggtat ctgcgctctg ctgaagccag 840 |
| | ttaccttcgg aaaaagagtt ggtagctctt gatccggcaa acaaaccacc gctggtagcg 900 |
| | gtggtttttt tgtttgcaag cagcagatta cgcgcagaaa aaaaggatct caagaagatc 960 |
| | ctttgatctt ttctacgggg tctgacgctc agtggaacga aaactcacgt taagggattt 1020 |
| | tggtcatgag attatcaaaa aggatcttca cctagatcct tttaaattaa aaatgaagtt 1080 |
| | ttaaatcaat ctaaagtata tatgagtaaa cttggtctga cagttaccaa tgcttaatca 1140 |
| | gtgaggcacc tatctcagcg atctgtctat ttcgttcatc catagttgcc tggctccccg 1200 |
| | ttgtgtagat aactacgata cgggagggct taccatctgg ccccagtgct gcaatgatac 1260 |
| | cgcgagaccc acgctcaccg gctccagatt tatcagcaat aaaccagcca gccggaaggg 1320 |
| | ccgagcgcag aagtggtcct gcaactttat ccgcctccat ccagtctatt aattgttgcc 1380 |
| | gggaagctag agtaagtagt tcgccagtta atagtttgcg caacgttgtt gccattgcta 1440 |
| | caggcatcgt ggtgtcacgc tcgtcgtttg gtatggcttc attcagctcc ggttcccaac 1500 |
| | gatcaaggcg agttacatga tcccccatgt tgtgcaaaaa agcggttagc tccttcggtc 1560 |
| | ctccgatcgt tgtcagaagt aagttggccg cagtgttatc actcatggtt atggcagcac 1620 |
| | tgcataattc tcttactgtc atgccatccg taagatgctt ttctgtgact ggtgagtact 1680 |
| | caaccaagtc attctgagaa tagtgtatgc ggcgaccgag ttgctcttgc ccggcgtcaa 1740 |
| | tacgggataa taccgcgcca catagcagaa ctttaaaagt gctcatcatt ggaaaacgtt 1800 |
| | cttcggggcg aaaactctca aggatcttac cgctgttgag atccagttcg atgtaaccca 1860 |
| | ctcgtgcacc caactgatct tcagcatctt ttactttcac cagcgtttct gggtgagcaa 1920 |
| | aaacaggaag gcaaaatgcc gcaaaaaagg gaataagggc gacacggaaa tgttgaatac 1980 |
| | tcatactctt cctttttcaa tattattgaa gcatttatca gggttattgt ctcatgagcg 2040 |
| | gatacatatt tgaatgtatt tagaaaaata aacaaatagg ggttccgcgg gtaccactag 2100 |
| | tgacatc 2107 |
| 57 | tatatagatc tccgttacct cgacctgatg agctccaaaa agagcgaaac ctattaggtc 60 |
| | gtcgagtact gggttggaat tctcgggtgc caaggatagt actcagaaga caaccagaga 120 |
| | aacacacgtt gtggtatatt acctggtggc gcgccccgtt cagagttcta cagtccgacg 180 |
| | atcgcggccg cgtcgacgga tccaagcttc ctgcaggggt gtgccgtggc tgacaggaaa 240 |
| | ctgtcagcca ctgtccgtcg agtctccgta tgagactcag aagacaaacg gcgaaacaca 300 |
| | ccttgtgtgg tatattaccc gtttctagac tgaggttgat gtcactagtg ctagacgcgt 360 |
| | tgctggcgtt tttccatagg ctccgccccc ctgacgagca tcacaaaaat cgacgctcaa 420 |
| | gtcagaggtg gcgaaacccg acaggactat aaagatacca ggcgtttccc cctggaagct 480 |
| | ccctcgtgcg ctctcctgtt ccgaccctgc cgcttaccgg atacctgtcc gcctttctcc 540 |
| | cttcgggaag cgtggcgctt tctcatagct cacgctgtag gtatctcagt tcggtgtagg 600 |
| | tcgttcgctc caagctgggc tgtgtgcacg aaccccccgt tcagcccgac cgctgcgcct 660 |
| | tatccggtaa ctatcgtctt gagtccaacc cggtaagaca cgacttatcg ccactggcag 720 |
| | cagccactgg taacaggatt agcagagcga ggtatgtagg cggtgctaca gagttcttga 780 |
| | agtggtggcc taactacggc tacactagaa gaacagtatt tggtatctgc gctctgctga 840 |
| | agccagttac cttcggaaaa agagttggta gctcttgatc cggcaaacaa accaccgctg 900 |
| | gtagcggtgg tttttttgtt tgcaagcagc agattacgcg cagaaaaaaa ggatctcaag 960 |
| | aagatccttt gatcttttct acggggtctg acgctcagtg gaacgaaaac tcacgttaag 1020 |
| | ggattttggt catgagatta tcaaaaagga tcttcaccta gatcctttta aattaaaaat 1080 |
| | gaagttttaa atcaatctaa agtatatatg agtaaacttg gtctgacagt taccaatgct 1140 |
| | taatcagtga ggcacctatc tcagcgatct gtctatttcg ttcatccata gttgcctggc 1200 |
| | tccccgttgt gtagataact acgatacggg agggcttacc atctggcccc agtgctgcaa 1260 |
| | tgataccgcg agacccacgc tcaccggctc cagatttatc agcaataaac cagccagccg 1320 |
| | gaagggccga gcgcagaagt ggtcctgcaa ctttatccgc ctccatccag tctattaatt 1380 |
| | gttgccggga agctagagta agtagttcgc cagttaatag tttgcgcaac gttgttgcca 1440 |
| | ttgctacagg catcgtggtg tcacgctcgt cgtttggtat ggcttcattc agctccggtt 1500 |
| | cccaacgatc aaggcgagtt acatgatccc ccatgttgtg caaaaaagcg gttagctcct 1560 |
| | tcggtcctcc gatcgttgtc agaagtaagt tggccgcagt gttatcactc atggttatgg 1620 |
| | cagcactgca taattctctt actgtcatgc catccgtaag atgcttttct gtgactggtg 1680 |
| | agtactcaac caagtcattc tgagaatagt gtatgcggcg accgagttgc tcttgcccgg 1740 |
| | cgtcaatacg ggataatacc gcgccacata gcagaacttt aaaagtgctc atcattggaa 1800 |
| | aacgttcttc ggggcgaaaa ctctcaagga tcttaccgct gttgagatcc agttcgatgt 1860 |
| | aacccactcg tgcacccaac tgatcttcag catcttttac tttcaccagc gtttctgggt 1920 |
| | gagcaaaaac aggaaggcaa aatgccgcaa aaaagggaat aagggcgaca cggaaatgtt 1980 |
| | gaatactcat actcttcctt tttcaatatt attgaagcat ttatcagggt tattgtctca 2040 |
| | tgagcggata catatttgaa tgtatttaga aaaataaaca aataggggtt ccgcgggtac 2100 |
| | cactagtgac atc 2113 |
| 58 | cgtactcgac ggacagtggc tgacagtttc ctgtcagcca cggcacaccc ctg |
| 59 | tatatagatc tccgttacct cgacctgatg agctccaaaa agagcgaaac ctattaggtc 60 |
| | gtcgagtact gggttggaat tctcgggtgc caaggatagt actcagaaga caaccagaga 120 |
| | aacacacgtt gtggtatatt acctggtggc gcgccccgtt cagagttcta cagtccgacg 180 |
| | atcgcggccg cgtcgacgga tccaagcttc ctgcaggggt gtgccgtggc tgacaggaaa 240 |
| | ctgtcagcca ctgtccgtcg agtacgtcta gaatata 277 |
| 60 | ggcgcgcccc |
| 61 | ggggtgtgcc |
| 62 | ggggugugcc |

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

What is claimed is:

1. A nucleic acid molecule comprising, in 5′ to 3′ order:
(i) an upstream ribozyme catalytic core,
(ii) an upstream cleavage site,
(iii) a central ribozyme catalytic core,
(iv) a downstream cleavage site, and
(v) a downstream ribozyme catalytic core;
wherein the nucleic acid molecule further comprises a sequence of interest between (ii) and (iv),
wherein the upstream ribozyme catalytic core is configured to cleave the upstream cleavage site to produce an upstream cleaved terminus and the downstream ribozyme catalytic core is configured to cleave the downstream cleavage site to produce a downstream cleaved terminus, and
wherein the central ribozyme catalytic core is configured to join the upstream cleaved terminus and the downstream cleaved terminus to produce a circular nucleic acid molecule comprising the sequence of interest.

2. The nucleic acid molecule of claim 1, wherein the upstream ribozyme catalytic core is a self-cleaving ribozyme catalytic core.

3. The nucleic acid molecule of claim 1, wherein the downstream ribozyme catalytic core is a self-cleaving ribozyme catalytic core.

4. The nucleic acid molecule of any one of claims 1 to 3, wherein the upstream ribozyme catalytic core is a hammerhead ribozyme catalytic core.

5. The nucleic acid molecule of claim 4, wherein the hammerhead ribozyme catalytic core is a Schistosoma mansoni hammerhead (HH), a peach latent mosaic viroid hammerhead (HHl), a Homo sapiens HH9, or variants thereof.

6. The nucleic acid molecule of any one of claims 1 to 3, wherein the upstream ribozyme catalytic core is a hairpin ribozyme catalytic core.

7. The nucleic acid molecule of claim 6, wherein the hairpin ribozyme catalytic core is from satellite arabis mosaic virus RNA, satellite tobacco ringspot virus RNA, satellite chicory yellow mottle virus RNA, or variants thereof.

8. The nucleic acid molecule of any one of claims 1 to 3, wherein the upstream catalytic core is a VS catalytic core, a twister catalytic core, a twister sister catalytic core, a hatchet catalytic core or a pistol catalytic core.

9. The nucleic acid molecule of any one of claims 1 to 8, wherein the downstream ribozyme catalytic core is a hammerhead ribozyme catalytic core.

10. The nucleic acid molecule of claim 9, wherein the hammerhead ribozyme catalytic core is a Schistosoma mansoni HH, a peach latent mosaic viroid HH, a Homo sapiens HH9, or variants thereof.

11. The nucleic acid molecule of any one of claims 1 to 8, wherein the downstream ribozyme catalytic core is a hairpin ribozyme catalytic core.

12. The nucleic acid molecule of claim 11, wherein the hairpin ribozyme catalytic core from satellite arabis mosaic virus RNA, satellite tobacco ringspot virus RNA, satellite chicory yellow mottle virus RNA, or variants thereof.

13. The nucleic acid molecule of any one of claims 1 to 8, wherein the downstream ribozyme catalytic core is a HDV ribozyme catalytic core.

14. The nucleic acid molecule of claim 13, wherein the HDV ribozyme catalytic core is from a HDV genome, HDV antigenome, or variants thereof.

15. The nucleic acid molecule of any one of claims 1 to 8, wherein the downstream catalytic core is a VS catalytic core, a twister catalytic core, a twister sister catalytic core, a hatchet catalytic core or a pistol catalytic core.

16. The nucleic acid molecule of any one of claims 1 to 3, wherein the upstream ribozyme catalytic core is a hammerhead ribozyme catalytic core and the downstream ribozyme catalytic core is a hammerhead ribozyme catalytic core.

17. The nucleic acid molecule of any one of claims 1 to 3, wherein the upstream ribozyme catalytic core is a hairpin ribozyme catalytic core and the downstream ribozyme catalytic core is a hairpin ribozyme catalytic core.

18. The nucleic acid molecule of any one of claims 1 to 3, wherein the upstream ribozyme catalytic core is a hammerhead ribozyme catalytic core and the downstream ribozyme catalytic core is a hairpin ribozyme catalytic core.

19. The nucleic acid molecule of any one of claims 1 to 3, wherein the upstream ribozyme catalytic core is a hammerhead ribozyme catalytic core and the downstream ribozyme catalytic core is a HDV ribozyme catalytic core.

20. The nucleic acid molecule of any one of claims 1 to 3, wherein the upstream ribozyme catalytic core is a hairpin ribozyme catalytic core and the downstream ribozyme catalytic core is a hammerhead ribozyme catalytic core.

21. The nucleic acid molecule of any one of claims 1 to 3, wherein the upstream ribozyme catalytic core is a hairpin ribozyme catalytic core and the downstream ribozyme catalytic core is a HDV ribozyme catalytic core.

22. A nucleic acid molecule comprising, in 5′ to 3′ order:
(i) an upstream ribozyme catalytic core,
(ii) an upstream cleavage site,
(iii) a central ribozyme catalytic core, and
(iv) a downstream cleavage site,
wherein the nucleic acid molecule further comprises a sequence of interest between (ii) and (iv),
wherein the upstream ribozyme catalytic core is configured to cleave the upstream cleavage site to produce an upstream cleaved terminus and the central ribozyme catalytic core is configured to cleave the downstream cleavage site to produce a downstream cleaved terminus, and
wherein the central ribozyme catalytic core is configured to join the upstream cleaved terminus and the downstream cleaved terminus to produce a circular nucleic acid molecule comprising the sequence of interest.

23. The nucleic acid molecule of claim 22, wherein the upstream ribozyme catalytic core is a self-cleaving ribozyme catalytic core.

24. The nucleic acid molecule of claim 22 or 23, wherein the upstream ribozyme catalytic core is a hammerhead ribozyme catalytic core.

25. The nucleic acid molecule of claim 24, wherein the hammerhead ribozyme catalytic core is a Schistosoma mansoni HH, a peach latent mosaic viroid HH, a Homo sapiens HH9, or variants thereof.

26. The nucleic acid molecule of claim 22 or 23, wherein the upstream ribozyme catalytic core is a hairpin ribozyme catalytic core.

27. The nucleic acid molecule of claim 26, wherein the hairpin ribozyme catalytic core is from satellite arabis mosaic virus RNA, satellite tobacco ringspot virus RNA, satellite chicory yellow mottle virus RNA, or variants thereof.

28. The nucleic acid molecule of any one of claims 22 to 27, wherein the upstream catalytic core is a VS catalytic core, a twister catalytic core, a twister sister catalytic core, a hatchet catalytic core or a pistol catalytic core.

29. A nucleic acid molecule comprising, in 5′ to 3′ order:
(i) an upstream cleavage site,
(ii) a central ribozyme catalytic core,
(iii) a downstream cleavage site, and
(iv) a downstream ribozyme catalytic core,
wherein the nucleic acid molecule further comprises a sequence of interest between (i) and (iii),
wherein the central ribozyme catalytic core is configured to cleave the upstream cleavage site to produce an upstream cleaved terminus and the downstream ribozyme catalytic core is configured to cleave the downstream cleavage site to produce a downstream cleaved terminus, and
wherein the central ribozyme catalytic core is configured to join the upstream cleaved terminus and the downstream cleaved terminus to produce a circular nucleic acid molecule comprising the sequence of interest.

30. The nucleic acid molecule of claim 29, wherein the downstream ribozyme catalytic core is a self-cleaving ribozyme catalytic core.

31. The nucleic acid molecule of claim 29 or 30, wherein the downstream ribozyme catalytic core is a hammerhead ribozyme catalytic core.

32. The nucleic acid molecule of claim 31, wherein the hammerhead ribozyme catalytic core is a Schistosoma mansoni HH, a peach latent mosaic viroid HH, a Homo sapiens HH9, or variants thereof.

33. The nucleic acid molecule of claim 29 or 30, wherein the downstream ribozyme catalytic core is a hairpin ribozyme catalytic core.

34. The nucleic acid molecule of claim 33, wherein the hairpin ribozyme catalytic core is from satellite arabis mosaic virus RNA, satellite tobacco ringspot virus RNA, satellite chicory yellow mottle virus RNA, or variants thereof.

35. The nucleic acid molecule of claim 29 or 30, wherein the downstream ribozyme catalytic core is a HDV ribozyme catalytic core.

36. The nucleic acid molecule of claim 35, wherein the HDV ribozyme catalytic core is from a HDV genome, a HDV antigenome, or variants thereof.

37. The nucleic acid molecule of any one of claims 29 to 36, wherein the downstream catalytic core is a VS catalytic core, a twister catalytic core, a twister sister catalytic core, a hatchet catalytic core or a pistol catalytic core.

38. The nucleic acid molecule of any one of claims 1 to 37, wherein the central ribozyme catalytic core is a ribozyme catalytic core that catalyzes reversible cleavage.

39. The nucleic acid molecule of any one of claims 1 to 38, wherein the central ribozyme catalytic core is a central hairpin catalytic core.

40. The nucleic acid molecule of claim 39, wherein the central hairpin ribozyme catalytic core is from satellite arabis mosaic virus RNA, satellite tobacco ringspot virus RNA, satellite chicory yellow mottle virus RNA, or variants thereof.

41. The nucleic acid molecule of any one of claims 1 to 38, wherein the central ribozyme catalytic core is a central VS ribozyme catalytic core.

42. The nucleic acid molecule of any one of claims 1 to 41, wherein the sequence of interest is located between the upstream cleavage site and the central ribozyme catalytic core.

43. The nucleic acid molecule of any one of claims 1 to 41, wherein the sequence of interest is located between the central ribozyme catalytic core and the downstream cleavage site.

44. The nucleic acid molecule of any one of claims 1 to 43, wherein the sequence of interest comprises internal ribozyme entry site (IRES).

45. The nucleic acid molecule of any one of claims 1 to 43, wherein the sequence of interest comprises an interfering RNA molecule.

46. The nucleic acid molecule of claim 45, wherein the interfering RNA molecule is a siRNA.

47. The nucleic acid molecule of claim 45, wherein the interfering RNA molecule is a shRNA.

48. The nucleic acid molecule of any one of claims 1 to 43, wherein the sequence of interest comprises a miRNA.

49. The nucleic acid molecule of any one of claims 1 to 43, wherein the sequence of interest comprises a gRNA (e.g., a sgRNA)

50. The nucleic acid molecule of any one of claims 1 to 43, wherein the nucleic acid sequence of interest comprises a miRNA binding site.

51. The nucleic acid molecule of any one of claims 1 to 43, wherein the nucleic acid sequence of interest comprises an antagomir.

52. The nucleic acid molecule of any one of claims 1 to 43, wherein the nucleic acid sequence of interest comprises an aptamer.

53. The nucleic acid molecule of any one of claims 1 to 43, wherein the nucleic acid sequence of interest comprises a sequence encoding a reporter.

54. The nucleic acid molecule of any one of claims 1 to 43, wherein the nucleic acid sequence of interest comprises a sequence encoding a protein.

55. The nucleic acid molecule of claim 54, wherein the protein is a therapeutic protein.

56. The nucleic acid molecule of any one of claims 1 to 43, wherein the nucleic acid sequence of interest comprises a sequence that binds a RNA binding protein.

57. The nucleic acid molecule of any one of claims 1 to 43, wherein the nucleic acid sequence of interest comprises a spacer sequence.

58. The nucleic acid molecule of any one of claims 1 to 43, wherein the nucleic acid sequence of interest comprises a translation regulation motif.

59. The nucleic acid molecule of any one of claims 1 to 43, wherein the sequence of interest is at least 250 nucleotides in length.

60. The nucleic acid molecule of any one of claims 1 to 43, wherein the sequence of interest is at least 500 nucleotides in length.

61. The nucleic acid molecule of any one of claims 1 to 43, wherein the sequence of interest is at least 1000 nucleotides in length.

62. The nucleic acid molecule of any one of claims 1 to 43, wherein the sequence of interest is at least 1500 nucleotides in length.

63. The nucleic acid molecule of any one of claims 1 to 43, wherein the sequence of interest is at least 2000 nucleotides in length.

64. The nucleic acid molecule of any one of claims 1 to 43, wherein the sequence of interest is at least 2500 nucleotides in length.

65. The nucleic acid molecule of any one of claims 1 to 64, wherein the nucleic acid molecule further comprises a first hairpin insulator sequence and a second hairpin insulator sequence, wherein the first hairpin insulator sequence and the second hairpin insulator sequence are complementary.

66. The nucleic acid molecule of claim 65, wherein each hairpin insulator sequence is 10 base pairs long.

67. The nucleic acid molecule of claim 65 or 66, wherein the first hairpin insulator sequence upstream of the sequence of interest.

68. The nucleic acid molecule of claim 65 or 66, wherein the second hairpin insulator sequence is downstream of the sequence of interest.

69. The nucleic acid molecule of any one of claims 1 to 68, wherein the nucleic acid molecule further comprises an 11 base pair stem between the sequence of interest and the downstream cleavage site.

70. The nucleic acid molecule of any one of claims 1 to 69, wherein the nucleic acid molecule further comprises a binding sequence.

71. The nucleic acid molecule of claim 70, wherein the binding sequence is upstream of the central ribozyme hairpin catalytic core.

72. The nucleic acid molecule of claim 70 or 71, wherein the binding site is a primer for reverse transcription, a RNA polymerase binding site, a transcription factor binding site, and/or combinations thereof.

73. The nucleic acid molecule of any one of claims 1 to 72, wherein the nucleic acid molecule further comprises a promoter.

74. The nucleic acid molecule of claim 73, wherein the promoter is an RNA polymerase promoter.

75. The nucleic acid molecule of any one of claims 1 to 74, wherein the nucleic acid molecule comprises RNA.

76. The nucleic acid molecule of any one of claims 1 to 74, wherein the nucleic acid molecule comprises DNA.

77. The nucleic acid molecule of any one of claims 1 to 74, wherein the nucleic acid molecule comprises modified nucleotides.

78. A construct comprising the nucleic acid molecule of any one of claims 1 to 77.

79. A circular nucleic acid molecule produced by any one of the nucleic acid molecules according to any one of claims 1 to 77.

80. A cell comprising the nucleic acid molecule according to any one of claims 1 to 77.

81. A method of generating circular nucleic acid molecules, the method comprising expressing a nucleic acid molecule according to any one of claims 1 to 77 in a cell.


SEQUENCE LISTING (104 sequences)

SEQ ID NO: 1 - DNA, 43 bp, synthetic construct
  source 1..43: mol_type = other DNA
caggggtgtgccgtggctgacaggaaactgtcagccactgtcc

SEQ ID NO: 2 - DNA, 39 bp, synthetic construct
  source 1..39: mol_type = other DNA
caggggtgtgccgtggctgacgaaagtcagccactgtcc

SEQ ID NO: 3 - DNA, 35 bp, synthetic construct
  source 1..35: mol_type = other DNA
caggggtgtgccgtggctggaaacagccactgtcc

SEQ ID NO: 4 - DNA, 27 bp, synthetic construct
  source 1..27: mol_type = other DNA
ggtgtgccgtggcgaaagccactgtcc

SEQ ID NO: 5 - DNA, 11 bp, synthetic construct
  source 1..11: mol_type = other DNA
gtcgagtctcc

SEQ ID NO: 6 - DNA, 12 bp, synthetic construct
  source 1..12: mol_type = other DNA
gtcgagtatcgg

SEQ ID NO: 7 - DNA, 14 bp, synthetic construct
  source 1..14: mol_type = other DNA
gtcgagtccaatcc

SEQ ID NO: 8 - DNA, 2083 bp, synthetic construct
  source 1..2083: mol_type = other DNA
ctcgaggctagctgtccgtcgagtctccgttggaattctcgggtgccaaggatgagactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgggtcggcatggcatctccacctcctcgcggtccgacctgggcatccgaaggaggacgcacgtccactcggatggctaagggagagccatctagagatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtaccactagtgacatc

SEQ ID NO: 9 - DNA, 52 bp, synthetic construct
  source 1..52: mol_type = other DNA
ctctctcgagtaatacgactcactatagggtgtccgtcgagtctccgttgga

SEQ ID NO: 10 - DNA, 56 bp, synthetic construct
  source 1..56: mol_type = other DNA
acggagactcgacggacagtggctgacagtttcctgtcagccacggcacacccctg

SEQ ID NO: 11 - DNA, 165 bp, Homo sapiens
  source 1..165: mol_type = genomic DNA
agtgcgagctcgagccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgcctgaggtt

SEQ ID NO: 12 - DNA, 288 bp, synthetic construct
  source 1..288: mol_type = other DNA
taatacgactcactataggagtgcgagctcgagccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtact

SEQ ID NO: 13 - DNA, 37 bp, synthetic construct
  source 1..37: mol_type = other DNA
taatacgactcactataggagtgcgagctcgagccgt

SEQ ID NO: 14 - DNA, 53 bp, synthetic construct
  source 1..53: mol_type = other DNA
agtactcgacggacagtggctgacagtttcctgtcagccacggcacacccctg

SEQ ID NO: 15 - DNA, 291 bp, synthetic construct
  source 1..291: mol_type = other DNA
taatacgactcactataggagtgcgagctcgagccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtctccgt

SEQ ID NO: 16 - DNA, 291 bp, synthetic construct
  source 1..291: mol_type = other DNA
taatacgactcactataggagtgcgagctcgagccgttacctcgacctgatgagctccaaaaagagcgaaacctattaggtcgtcgagtactgggttggaattctcgggtgccaaggatgagactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtctccgt

SEQ ID NO: 17 - DNA, 185 bp, synthetic construct
  source 1..185: mol_type = other DNA
agtgcgagctcgaggagactcagaagacaaacggcgaaacacaccttgtgtggtatattacccgttggagattccagaggattggttacctatctcccatgcccatgtcggcattgtccgtcgagtctccgttggaattctcgggtgccaaggatgagactcagaagacaaccagagaaacacac

SEQ ID NO: 18 - DNA, 37 bp, synthetic construct
  source 1..37: mol_type = other DNA
taatacgactcactataggagtgcgagctcgaggaga

SEQ ID NO: 19 - DNA, 346 bp, synthetic construct
  source 1..346: mol_type = other DNA
taatacgactcactataggagtgcgagctcgaggagactcagaagacaaacggcgaaacacaccttgtgtggtatattacccgttggagattccagaggattggttacctatctcccatgcccatgtcggcattgtccgtcgagtctccgttggaattctcgggtgccaaggatgagactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtctccgt

SEQ ID NO: 20 - DNA, 297 bp, synthetic construct
  source 1..297: mol_type = other DNA
taatacgactcactataggagtgcgagctcgagggatgtacctcgacctgatgagtcccaaataggacgaaacgccgaaaggcgtcgtcgagtccaatccgttggaattctcgggtgccaaggattggactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtctccgt

SEQ ID NO: 21 - DNA, 171 bp, Schistosoma mansoni
  source 1..171: mol_type = genomic DNA
agtgcgagctcgagggatgtacctcgacctgatgagtcccaaataggacgaaacgccgaaaggcgtcgtcgagtccaatccgttggaattctcgggtgccaaggattggactcagaagacaaccagagaaacacacgttgtggtatattacctggtggcgcgcctgaggtt

SEQ ID NO: 22 - DNA, 37 bp, synthetic construct
  source 1..37: mol_type = other DNA
taatacgactcactataggagtgcgagctcgagggat

SEQ ID NO: 23 - DNA, 297 bp, synthetic construct
  source 1..297: mol_type = other DNA
taatacgactcactataggagtgcgagctcgagggatgtacctcgacctgatgagtcccaaataggacgaaacgccgaaaggcgtcgtcgagtccaatccgttggaattctcgggtgccaaggatgagactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtctccgt

SEQ ID NO: 24 - DNA, 2135 bp, synthetic construct
  source 1..2135: mol_type = other DNA
tatatagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgggtcggcatggcatctccacctcctcgcggtccgacctgggcatccgaaggaggacgcacgtccactcggatggctaagggagagccatctagactgaggttgatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtaccactagtgacatc

SEQ ID NO: 25 - DNA, 288 bp, synthetic construct; identical to SEQ ID NO: 12
  source 1..288: mol_type = other DNA

SEQ ID NO: 26 - DNA, 32 bp, synthetic construct
  source 1..32: mol_type = other DNA
tatatagatctccgttacctcgacctgatgag

SEQ ID NO: 27 - DNA, 39 bp, synthetic construct
  source 1..39: mol_type = other DNA
cgtctagatggctctcccttagccatccgagtggacgtg

SEQ ID NO: 28 - DNA, 2284 bp, synthetic construct
  source 1..2284: mol_type = other DNA
actagtgacatcatgtcgatatcctatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcgatatcgacatgatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtacc

SEQ ID NO: 29 - DNA, 2173 bp, synthetic construct
  source 1..2173: mol_type = other DNA
taatacgactcactataggagtgcgagctcgagccgttacctcgacctgatgagctccaaaaagagcgaaacctattaggtcgtcgagtactgggttggaattctcgggtgccaaggatagtactcagaaaccgaccagagaaacacacgttgtggtatattacctggtggcgcgcccgttcagagttctacagtccgacgatcgcggccgcgtcgacggatccaagcttcctgcaggggtgtgccgtggctgacaggaaactgtcagccaccggtagtcgagtctccgttctagactgaggttgatgtcactagtgctagacgcgttgctggcgtttttccataggctccgcccccctgacgagcatcacaaaaatcgacgctcaagtcagaggtggatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtaccactagtgacatc

SEQ ID NO: 30 - DNA, 2007 bp, synthetic construct
  source 1..2007: mol_type = other DNA
ctcgaggctagctgtccgtcgagtctccgttggaattctcgggtgccaaggatgagactcagaaaccgaccagagaaacacacgttgtggtatattacctggtggcgcgccccgttcagagttctacagtccgacgatcgcggccgcgtcgacggatccaagcttcctgcagggctgntccgtggctgacaggaaactgtcagccaccggtagtcgagtctccgttctagagatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtaccactagtgacatc

SEQ ID NO: 31 - DNA, 45 bp, synthetic construct
  source 1..45: mol_type = other DNA
atcggccggcccgcggaacccctatttgtttatttttctaaatac

SEQ ID NO: 32 - DNA, 44 bp, synthetic construct
  source 1..44: mol_type = other DNA
gggttggaattctcgggtgccaaggatagtactcagaaaccgac

SEQ ID NO: 33 - DNA, 24 bp, synthetic construct
  source 1..24: mol_type = other DNA
cacctctgacttgagcgtcgattt

SEQ ID NO: 34 - DNA, 270 bp, synthetic construct
  source 1..270: mol_type = other DNA
taatacgactcactataggagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggcgaaagccactgtccgtcgagtactgg

SEQ ID NO: 35 - DNA, 46 bp, synthetic construct
  source 1..46: mol_type = other DNA
taatacgactcactataggagatctccgttacctcgacctgatgag

SEQ ID NO: 36 - DNA, 39 bp, synthetic construct
  source 1..39: mol_type = other DNA
ccagtactcgacggacagtggctttcgccacggcacacc

SEQ ID NO: 37 - DNA, 274 bp, synthetic construct
  source 1..274: mol_type = other DNA
taatacgactcactataggagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctggaaacagccactgtccgtcgagtactgg

SEQ ID NO: 38 - DNA, 47 bp, synthetic construct
  source 1..47: mol_type = other DNA
ccagtactcgacggacagtggctgtttccagccacggcacacccctg

SEQ ID NO: 39 - DNA, 278 bp, synthetic construct
  source 1..278: mol_type = other DNA
taatacgactcactataggagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacgaaagtcagccactgtccgtcgagtactgg

SEQ ID NO: 40 - DNA, 51 bp, synthetic construct
  source 1..51: mol_type = other DNA
ccagtactcgacggacagtggctgactttcgtcagccacggcacacccctg

SEQ ID NO: 41 - DNA, 282 bp, synthetic construct
  source 1..282: mol_type = other DNA
taatacgactcactataggagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtactgg

SEQ ID NO: 42 - DNA, 55 bp, synthetic construct
  source 1..55: mol_type = other DNA
ccagtactcgacggacagtggctgacagtttcctgtcagccacggcacacccctg

SEQ ID NO: 43 - DNA, 136 bp, synthetic construct
  source 1..136: mol_type = other DNA
agtgcgagcctgcaggggtgtgccgtggctgacaggaaactgtcagccactgtccgtcgagtctccgtatgagactcagaagacaaacggcgaaacacaccttgtgtggtatattacccgtttctagactgaggtt

SEQ ID NO: 44 - DNA, 23 bp, synthetic construct
  source 1..23: mol_type = other DNA
aacctcagtctagaaacgggtaa

SEQ ID NO: 45 - DNA, 351 bp, synthetic construct
  source 1..351: mol_type = other DNA
taatacgactcactataggagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtctccgtatgagactcagaagacaaacggcgaaacacaccttgtgtggtatattacccgtttctagactgaggtt

SEQ ID NO: 46 - DNA, 130 bp, synthetic construct
  source 1..130: mol_type = other DNA
agtgcgagcctgcaggggtgtgccgtggctgacaggaaactgtcagccaccggtcctggtatccaatccgaaaggatgtacctcgacctgatgagtcccaaataggacgaaaccggtctagactgaggtt

SEQ ID NO: 47 - DNA, 22 bp, synthetic construct
  source 1..22: mol_type = other DNA
aacctcagtctagaccggtttc

SEQ ID NO: 48 - DNA, 353 bp, synthetic construct
  source 1..353: mol_type = other DNA
taatacgactcactataggagtgcgagctcgagccgttacctcgacctgatgagctccaaaaagagcgaaacctattaggtcgtcgagtactgggttggaattctcgggtgccaaggatagtactcagaaaccgaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccaccggtcctggtatccaatccgaaaggatgtacctcgacctgatgagtcccaaataggacgaaaccggtctagactgaggtt

SEQ ID NO: 49 - DNA, 53 bp, synthetic construct
  source 1..53: mol_type = other DNA
agtactcgacgaccggtggctgacagtttcctgtcagccacggcacacccctg

SEQ ID NO: 50 - DNA, 289 bp, synthetic construct
  source 1..289: mol_type = other DNA
taatacgactcactatagggctcgaggctagccggtcgtcgagtctccgttggaattctcgggtgccaaggatgagactcagaaaccgaccagagaaacacacgttgtggtatattacctggtggcgcgcccgttcagagttctacagtccgacgatcgcggccgcgtcgacggatccaagcttcctgcaggggtgtgccgtggctgacaggaaactgtcagccaccggtctgtgctaagcacactgatgagtctctgaaatgagacgaaaccggtctagactgaggtt

SEQ ID NO: 51 - DNA, 113 bp, synthetic construct
  source 1..113: mol_type = other DNA
agtgcgagcctgcaggggtgtgccgtggctgacaggaaactgtcagccaccggtctgtgctaagcacactgatgagtctctgaaatgagacgaaaccggtctagactgaggtt

SEQ ID NO: 52 - DNA, 45 bp, synthetic construct
  source 1..45: mol_type = other DNA
taatacgactcactatagggctcgaggctagccggtcgtcgagtc

SEQ ID NO: 53 - DNA, 336 bp, synthetic construct
  source 1..336: mol_type = other DNA
taatacgactcactataggagtgcgagctcgagccgttacctcgacctgatgagctccaaaaagagcgaaacctattaggtcgtcgagtactgggttggaattctcgggtgccaaggatagtactcagaaaccgaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccaccggtctgtgctaagcacactgatgagtctctgaaatgagacgaaaccggtctagactgaggtt

SEQ ID NO: 54 - DNA, 2053 bp, synthetic construct
  source 1..2053: mol_type = other DNA
tatatagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtacgtctagaatatagatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtaccactagtgacatc

SEQ ID NO: 55 - DNA, 64 bp, synthetic construct
  source 1..64: mol_type = other DNA
tatattctagacgtactcgacggacagtggctgacagtttcctgtcagccacggcacacccctg

SEQ ID NO: 56 - DNA, 2107 bp, synthetic construct
  source 1..2107: mol_type = other DNA
tatatagatctccgttacctcgacctgatgagctccaaaaagagcgaaacctattaggtcgtcgagtactgggttggaattctcgggtgccaaggatagtactcagaaaccgaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccaccggtcctggtatccaatccgaaaggatgtacctcgacctgatgagtcccaaataggacgaaaccggtctagactgaggttgatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtaccactagtgacatc

SEQ ID NO: 57 - DNA, 2113 bp, synthetic construct
  source 1..2113: mol_type = other DNA
tatatagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtctccgtatgagactcagaagacaaacggcgaaacacaccttgtgtggtatattacccgtttctagactgaggttgatgtcactagtgctagacgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctggctccccgttgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgggtaccactagtgacatc

SEQ ID NO: 58 - DNA, 53 bp, synthetic construct
  source 1..53: mol_type = other DNA
cgtactcgacggacagtggctgacagtttcctgtcagccacggcacacccctg

SEQ ID NO: 59 - DNA, 277 bp, synthetic construct
  source 1..277: mol_type = other DNA
tatatagatctccgttacctcgacctgatgagctccaaaaagagcgaaacctattaggtcgtcgagtactgggttggaattctcgggtgccaaggatagtactcagaagacaaccagagaaacacacgttgtggtatattacctggtggcgcgccccgttcagagttctacagtccgacgatcgcggccgcgtcgacggatccaagcttcctgcaggggtgtgccgtggctgacaggaaactgtcagccactgtccgtcgagtacgtctagaatata

SEQ ID NO: 60 - DNA, 10 bp, synthetic construct
  source 1..10: mol_type = other DNA
ggcgcgcccc

SEQ ID NO: 61 - DNA, 10 bp, synthetic construct
  source 1..10: mol_type = other DNA
ggggtgtgcc

SEQ ID NO: 62 - DNA, 10 bp, synthetic construct
  source 1..10: mol_type = other DNA
ggggtgtgcc

SEQ ID NO: 63 - DNA, 1738 bp, Coxsackievirus B3
  source 1..1738: mol_type = genomic DNA
ggatcctaatacgactcactataggagtgcgagctcgagccgttacctcgacctgatgagctccaaaaagagcgaaacctattaggtcgtcgagtactgggttgggaattctcgggtgccaaggatagtactcagaatgcgaccagagaaacacacgttgtggtatattacctggtacgccttgactagctacaccgccgcgcccacacgaaggctacaatctccacctccaggtggtggcgcgccccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggggtgtgccacgaggtagcagacccctacgctacgcagcacggttcactagtttacgtgtggctgacaggaaactgtcagccaccgcatgggtcggcatggcatctccacctcctcgcggtccgacctgggcatccgaaggaggacgcacgtccactcggatggctaagggagagccatctaga

SEQ ID NO: 64 - DNA, 595 bp, Bat picornavirus
  source 1..595: mol_type = genomic DNA
ccccccctccctccttttcatcggctgtccgttgccgtggagcttcactatccgtagtgttgttccggaaaacggacacagcaacaccttcctccgggttggtgcttgcaatccctttccccttttgttgacttcccggactcattcgttttggctttcacattggccctagaggctatcttgttggattcctgtaccctggcaatctgaactccttcgcatgccctcccacatggtcgaggacagtaaggcgcgggaccaagagagccgggcaccagacccatttctgaattggactgccaccgccaagaattgcggagcggctctgttgtgtgagatcctcctttcccacagatagcgcatcccagtgcgtgtgtatgggggttagggtgcatacagcatccgcgactgtggtcagatgagcggaagcatctgctaggataaaggggacgcgattcgctatccccgaagcatccctcattgaggtaaagccctagtaggtgacggtggactggaatgccaccctggttccgtgcagcttgacagagtgccggccggtgctctggccacagaaatcggctgttttgggacatgc

SEQ ID NO: 65 - DNA, 301 bp, Enterovirus sp.
  source 1..301: mol_type = genomic DNA
ttaccaatgcttaatcagtgaggcacctatctcagcgatctgtctatttcgttcatccatagttgcctgactccccgtcgtgtagataactacgatacgggagggcttaccatctggccccagtgcgcgtaagtctgggacggaaccgactactttgggtgtccgtgtttcctgttttacttactttggctgcttatggtgacaatctagtgttgttaccatatagctattggattggccatccggtgttttgaattgtgtgtttatactaattcttttacatatcacagacaaccaaatc

SEQ ID NO: 66 - DNA, 748 bp, Enterovirus sp.
  source 1..748: mol_type = genomic DNA
ttaaaacagcctgtgggttgttcccacccacagggcccaccgggcgccagcacactggtatcacggtacccttgtgcgcctgttttatccaccccttcccacagtaacttagaagcacaccatgtatacggtcaataggcggctcagtacaccaactgggccacgaccaagcacttctgttaccccggactgagtatcaataagctgctcacgcggctgaaggagaaaacgttcgttacccggccaattacttcgagaaacccagtaccaccatgaaggttgcgcagcgtttcgttccgcacaaccccagtgtagatcaggtcgatgagtcaccgcgttcctcacgggtgaccgtggcggtggctgcgttggcggcctgcccatggggcaacccatgggacgcttcaatactgacatggtgcgaagagtctattgagctaattggtagtcctccggcccctgaatgcggctaatcctaactgcggagcagatacccacaaaccagtgggcagtctgtcgtaacgggcaactctgcagcggaaccgactactttgggtgtccgtgtttccttttatccttacactggctgcttatggtgacaattgagaaattgttaccatatagctattggattggccatccggtgacaaacagagcagttgtttacctatttgttggtttcgtaccgctgaaccttaaagttctaaagaccctcaattttatcttagcacttaacacagcaaaatgc

SEQ ID NO: 67 - RNA, 301 nt, Homo sapiens
  source 1..301: mol_type = mRNA
ctcaccggctccagatttatcagcaataaaccagccagccggaagggccgagcgcagaagtggtcctgcaactttatccgccccaggcagtggggccggctggccagactcttgggggattccttagtgagtgagttcactgctcaaagaagggctttgccacttctgcagggaagccagccacgggccagcagtcttgaaagcgccacaagcagcagctgctgaggacttgaagtcagctgagggatttccgtaagcatcgtgatcgattagccttaagccttgaatccgagtcccatgc

SEQ ID NO: 68 - DNA, 502 bp, Guinea fowl picornavirus
  source 1..502: mol_type = genomic DNA
tgcatgtcatttctcccttccccctccaataaccttttccccctctaatcggactgatcaaccgtgtatgatgattattggattgtggagttatatgatggcctggcatggaatccaggaattcttgcatattggaattgtatcccacatgaacggggatgtggcaagtcccctagtagtgctgggctctacccaatgtgtaaggaccgtgacttcaagactagttatggtcgtgtgacaaagggaagaattttcttacccgcctgtgtgttaggtgcgcacgctgggaaaccataagcgaccaaaggtaaagtggtgctgaaatattgcaagctcaatgcctttgtcgtgagtcggtgccgctgtatctaatacacgcccccttcggggtaaatggctgacacgggcatccgcacttgggaatgtgcggctggaactagactgggtgatagcctgccggctggcgctgttgtcatggtatagccagttgattgccatatgc

SEQ ID NO: 69 - DNA, 610 bp, Macaca picornavirus
  source 1..610: mol_type = genomic DNA
ggaggatactttgtttagctttgcaattcttaaactgttttccatttcactggtcgtttgacgcttgtagggcgacaggtgttcctagctcttgcttttctaaactatcgaattttgttttccactcgttcatatgtctatgtatgaatgaacggggtgagtcctcgttggccctcgctggagtgtaaattcccagtctttctggaactagaattacacaagactccaggagtgttctgaagattttcatatttaaataaaatcttttgggattgtccttgatggttgtagcgatgtctagtgtgtgtgcggattcccatgctggcaacagcatcctcacaggccaaaagcccagggttaacagcccccgctagatgcatggtaccccccatgcccattttggatatgaaattaaggtttgtttacttaggaatttcataaggaagcttctgaggctatgaaggatgcccaggaggtacccgctatatgtggatctgacctggggaccccttgcgtagctttactgcgtagtgtgggtttaaaaagcgtctagtagccttgttttcgggggaccgaaaacttccacattattttgagactggcatcatgc

SEQ ID NO: 70 - DNA, 754 bp, Rabbit picornavirus
  source 1..754: mol_type = genomic DNA
ttccccttcccccttttcacggcccggccgcccgccgggtcgtcacccgttcagacacctacgggttatctgttcgggctataaatatgggcattcctcttcccccttccccttttgaagatgagtgcgcatattcttgactccgcctggattggccgcccaaggcgtgaacaagcagctaggccaccatgacactgcggtggtgtccgaacccgcgggtgccttcacgggcacctgtggtatgtaggactcccaccgtggtcttccctttccccctcaatctttccccctggttcgactaacgggaccagtgctggaacctgtccggtgaacggtatagcaggcccccccggcagaaacacccggtgcttaccccttaaggctagcccccttccatgaatttggttggggcaactagtgggtgtacagttggcgtgaaccctccggtctaggagtgctcttgcccaatcctctgtgtgtgccttgcagtagggactggcaatccttcgcgtaggtgatccgctgtgccatgccatcctggcgacaggaggcccagtgtgcgcaacctacgtcccttctgggtgctgcattgcattacctttggagtaagcttggtgtgccgaaaccccagggtttacgtaccactcgtggtgtgaggaatgtgccgcaggtaccccatccttgaggtgggatctgagcggtagctaattgtctagcaccactttcttccttttttctttgctggtcacgatgc

SEQ ID NO: 71 - DNA, 301 bp, Sudan ebolavirus
  source 1..301: mol_type = genomic DNA
ttaccaatgcttaatcagtgaggcacctatctcagcgatctgtctatttcgttcatccatagttgcctgactccccgtcgtgtagataactacgatacgggagggcttaccatctggccccagtgcataatatcctgatgaattctatagaacttaggattaagaaaaaattcatgatgaagattaaaaccttcatcatcctttaaaaagagagctattctttatctgaatgtccttattaatgtctaagagctattattttgtaccctcttagcctagacactgcccagcatataagccc

SEQ ID NO: 72 - DNA, 301 bp, Theilers murine encephalomyelitis virus
  source 1..301: mol_type = genomic DNA
ttaccaatgcttaatcagtgaggcacctatctcagcgatctgtctatttcgttcatccatagttgcctgactccccgtcgtgtagataactacgatacgggagggcttaccatctggccccagtgccttaagcgcctctgtagggaagccaggaatgtccaggaggtaccccttccgctcggaagggatctgacctggagacacatcacatgtgctttacacctgtgcttgtgtttaaaaattgtcgcagcttccccaaaccaagtggtcttggttttctctttttattatattgtcaatc

SEQ ID NO: 73 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 74 - DNA, 696 bp, synthetic construct
  source 1..696: mol_type = other DNA
atggtgagcaagggcgaggcagtgatcaaggagttcatgcggttcaaggtgcacatggagggctccatgaacggccacgagttcgagatcgagggcgagggcgagggccgcccctacgagggcacccagaccgccaagctgaaggtgaccaagggtggccccctgcccttctcctgggacatcctgtcccctcagttcatgtacggctccagggccttcatcaagcaccccgccgacatccccgactactataagcagtccttccccgagggcttcaagtgggagcgcgtgatgaacttcgaggacggcggcgccgtgaccgtgacccaggacacctccctggaggacggcaccctgatctacaaggtgaagctccgcggcaccaacttccctcctgacggccccgtaatgcagaagaagacaatgggctgggaagcgtccaccgagcggttgtaccccgaggacggcgtgctgaagggcgacattaagatggccctgcgcctgaaggacggcggccgctacctggcggacttcaagaccacctacaaggccaagaagcccgtgcagatgcccggcgcctacaacgtcgaccgcaagttggacatcacctcccacaacgaggactacaccgtggtggaacagtacgaacgctccgagggccgccactccaccggcggcatggacgagctgtacaag

SEQ ID NO: 75 - DNA, 277 bp, synthetic construct
  source 1..277: mol_type = other DNA
tgtccgtcgagtctccgttggaattctcgggtgccaaggatgagactcagaagacaaccagagaaacacacgttgtggtatattacctggtggcgcgccccgttcagagttctacagtccgacgatcgcggccgcgtcgacggatccaagcttcctgcaggggtgtgccgtggcgaaagccactgtccgggtcggcatggcatctccacctcctcgcggtccgacctgggcatccgaaggaggacgcacgtccactcggatggctaagggagagcca

SEQ ID NO: 76 - DNA, 1749 bp, synthetic construct
  source 1..1749: mol_type = other DNA
ctctctggatcctaatacgactcactataggagtgcgagctcgagccgttacctcgacctgatgagctccaaaaagagcgaaacctattaggtcgtcgagtactgggttgggaattctcgggtgccaaggatagtactcagaatgcgaccagagaaacacacgttgtggtatattacctggtacgccttgactagctacaccgccgcgcccacacgaaggctacaatctccacctccaggtggtggcgcgccccttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaacctgcaggggtgtgccacgaggtagcagacccctacgctacgcagcacggttcactagtttacgtgtggctgacaggaaactgtcagccaccgcatgggtcggcatggcatctccacctcctcgcggtccgacctgggcatccgaaggaggacgcacgtccactcggatggctaagggagagccatctagaagaga

SEQ ID NO: 77 - DNA, 41 bp, synthetic construct
  source 1..41: mol_type = other DNA
ctctctgctcttcacattttgctgtattcaacttaacaatg

SEQ ID NO: 78 - DNA, 22 bp, synthetic construct
  source 1..22: mol_type = other DNA
tgttttaaggggcgcgccacca

SEQ ID NO: 79 - DNA, 60 bp, synthetic construct
  source 1..60: mol_type = other DNA
ctctctggcgcgccataatatcctgatgaattctatagaacttaggattaagaaaaaatt

SEQ ID NO: 80 - DNA, 39 bp, synthetic construct
  source 1..39: mol_type = other DNA
ctctctgctcttcccatggcttatatgctgggcagtgtc

SEQ ID NO: 81 - DNA, 36 bp, synthetic construct
  source 1..36: mol_type = other DNA
ctctctggcgcgcccttaagcgcctctgtagggaag

SEQ ID NO: 82 - DNA, 56 bp, synthetic construct
  source 1..56: mol_type = other DNA
ctctctgctcttcccatattgacaatataataaaaagagaaaaccaagaccacttg

SEQ ID NO: 83 - DNA, 29 bp, synthetic construct
  source 1..29: mol_type = other DNA
ctctctggcgcgccccaggcagtggggcc

SEQ ID NO: 84 - DNA, 38 bp, synthetic construct
  source 1..38: mol_type = other DNA
ctctctgctcttcccatcatgggactcggattcaaggc

SEQ ID NO: 85 - DNA, 37 bp, synthetic construct
  source 1..37: mol_type = other DNA
ctctctggcgcgcctgcatgtcatttctcccttcccc

SEQ ID NO: 86 - DNA, 45 bp, synthetic construct
  source 1..45: mol_type = other DNA
ctctctgctcttcccatcatatggcaatcaactggctataccatg

SEQ ID NO: 87 - DNA, 50 bp, synthetic construct
  source 1..50: mol_type = other DNA
ctctctggcgcgccggaggatactttgtttagctttgcaattcttaaact

SEQ ID NO: 88 - DNA, 47 bp, synthetic construct
  source 1..47: mol_type = other DNA
ctctctgctcttcccatcatgatgccagtctcaaaataatgtggaag

SEQ ID NO: 89 - DNA, 35 bp, synthetic construct
  source 1..35: mol_type = other DNA
ctctctggcgcgccttccccttcccccttttcacg

SEQ ID NO: 90 - DNA, 44 bp, synthetic construct
  source 1..44: mol_type = other DNA
ctctctgctcttcccatcatcgtgaccagcaaagaaaaaaggaa

SEQ ID NO: 91 - DNA, 39 bp, synthetic construct
  source 1..39: mol_type = other DNA
ctctctggcgcgccttaaaacagcctgtgggttgttccc

SEQ ID NO: 92 - DNA, 53 bp, synthetic construct
  source 1..53: mol_type = other DNA
ctctctgctcttcccatcattttgctgtgttaagtgctaagataaaattgagg

SEQ ID NO: 93 - DNA, 32 bp, synthetic construct
  source 1..32: mol_type = other DNA
ctctctggcgcgccccccccctccctcctttt

SEQ ID NO: 94 - DNA, 42 bp, synthetic construct
  source 1..42: mol_type = other DNA
ctctctgctcttcccatcatgtcccaaaacagccgatttctg

SEQ ID NO: 95 - DNA, 41 bp, synthetic construct
  source 1..41: mol_type = other DNA
ctctctggcgcgccttaccaatgcttaatcagtgaggcacc

SEQ ID NO: 96 - DNA, 59 bp, synthetic construct
  source 1..59: mol_type = other DNA
ctctctgctcttcccatatttggttgtctgtgatatgtaaaagaattagtataaacaca

SEQ ID NO: 97 - DNA, 26 bp, synthetic construct
  source 1..26: mol_type = other DNA
gactaacctgcaggggtgtgccacga

SEQ ID NO: 98 - DNA, 41 bp, synthetic construct; identical to SEQ ID NO: 77
  source 1..41: mol_type = other DNA

SEQ ID NO: 99 - DNA, 29 bp, synthetic construct
  source 1..29: mol_type = other DNA
ctctctgctcttcaatggtgagcaagggc

SEQ ID NO: 100 - DNA, 36 bp, synthetic construct
  source 1..36: mol_type = other DNA
ctctctcctgcaggttacttgtacagctcgtccatg

SEQ ID NO: 101 - DNA, 10 bp, synthetic construct
  source 1..10: mol_type = other DNA
gtcgagtctc

SEQ ID NO: 102 - RNA, 358 nt, Tobacco ringspot virus
  source 1..358: mol_type = genomic RNA
acctccacctctaacatcggaacgacacccgcgccgccacatcgatcagttccgcatggtccattatatggtcttggacacaaagagaccaactgaagagacaaacaacacagtaaccaagggcctagagcgtaatcgccgctgccccataagagtatgctgtaccttcaactctctggcgcggagatgtgataggccgcgcccccgcttaggtttaacaagatcgggctatgggacagtggcctggtctaaaggccagactactcaggcactcctgctttgtcctgacagtccaccggctttcggtggtgctattgatcgcttggcacgacgcatccgatccccagaagctggagca

SEQ ID NO: 103 - RNA, 61 nt, Tobacco ringspot virus
  source 1..61: mol_type = genomic RNA
nnnnngaagnnnnnnnnngaaacnnnnnnnnnnnnnnanattannnnnnnnnyngdnnnnn

SEQ ID NO: 104 - RNA, 88 nt, Tobacco ringspot virus
  source 1..88: mol_type = genomic RNA
nnnnngaagnnnnnnnnngaaacnnnnnnnnnnnnnnanattannnnnnnnnnnnnnnnnnnnnnnnnnnnnnnnnnnnyngdnnnnn